(19) United States
(12) Patent Application Publication
(10) Pub. No.: US 2025/0268101 A1
(43) Pub. Date: Aug. 21, 2025

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Moon Sung KANG, Cheonan-si (KR); Jung Geun LEE, Cheonan-si (KR); Sang Yong PARK, Cheonan-si (KR)

(73) Assignee: DUK SAN NEOLUX CO., LTD., Cheonan-si (KR)

(21) Appl. No.: 19/057,277

(22) Filed: Feb. 19, 2025

(30) Foreign Application Priority Data
Feb. 19, 2024 (KR) 10-2024-0023290
Apr. 11, 2024 (KR) 10-2024-0048775

(51) Int. Cl. H10K 85/60; C07C 211/61; C07D 209/88; C07D 263/60; C07D 307/91; C07D 319/24; C07D 333/76; C07D 409/04; H10K 50/11; H10K 50/19

(52) U.S. Cl. CPC H10K 85/633; C07C 211/61; C07D 209/88; C07D 263/60; C07D 307/91; C07D 319/24; C07D 333/76; C07D 409/04; H10K 85/636; C07B 2200/05; C07C 2601/14; C07C 2602/10; C07C 2602/42; C07C 2603/18; C07C 2603/40; C07C 2603/48; C07C 2603/74; C07C 2603/97; H10K 50/11; H10K 50/19; H10K 85/615; H10K 85/622; H10K 85/626; H10K 85/6574; H10K 85/6576

(57) ABSTRACT

Provided are a compound of Formula 1 that can improve the luminous efficiency, stability, and lifespan of an organic electronic element using the same, the organic electronic element and an electronic device thereof.

### BACKGROUND

### Technical Field

The present invention relates to compounds for organic electronic elements, organic electronic elements using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer etc.

A material used as an organic material layer in an organic electronic element may be classified into a light emitting material and a charge transport material, such as a hole injection material, a hole transport material, an electron transport material, an electron injection material etc. depending on its function.

The most problematic issues with organic electroluminescent devices are lifespan and efficiency, and as displays become larger in size, these efficiency and lifespan issues must be resolved.

Efficiency, lifespan and driving voltage are related to each other, and when the efficiency is increased, the driving voltage is relatively decreased, and as the driving voltage is decreased, crystallization of organic materials due to Joule heating generated during driving decreases, and consequently, the lifespan tends to increase.

However, the efficiency cannot be maximized simply by improving the organic material layer. This is because, when the energy level and T1 value between each organic material layer, and the intrinsic properties (mobility, interfacial properties, etc.) of materials are optimally combined, long lifespan and high efficiency can be achieved at the same time.

Further, recently, in organic electroluminescent devices, in order to solve the emission problem in the hole transport layer, an emitting-auxiliary layer must be present between the hole transport layer and an emitting layer, and it is necessary to develop different emitting-auxiliary layers according to each of the emitting layers (R, G, B).

In general, electrons are transferred from the electron transport layer to the emitting layer, and holes are transferred from the hole transport layer to the emitting layer to generate excitons by recombination.

However, the material used for the hole transport layer has a low HOMO value and therefore has mostly low T1 value, therefore the exciton generated in the emitting layer is transferred to the hole transport layer, resulting in charge unbalance in the emitting layer, and light is emitted at the interface of the hole transport layer.

When light is emitted at the interface of the hole transport layer, the color purity and efficiency of the organic electronic element are lowered and the life span is shortened. Therefore, it is urgently required to develop an emitting-auxiliary layer having a high T1 value and a HOMO level between the HOMO energy level of the hole transport layer and the HOMO energy level of the emitting layer.

Meanwhile, it is necessary to develop a hole injection layer material having stable characteristics, that is, a high glass transition temperature, against Joule heating generated when the device is driven, while delaying penetration of the metal oxide from the anode electrode (ITO), which is one of the causes of shortening the lifespan of the organic electronic element, into the organic layer. The low glass transition temperature of the hole transport layer material has a characteristic that when the device is driven, the uniformity of the surface of the thin film is lowered, which has been reported to have a great influence on the lifespan of the device. In addition, OLED devices are mainly formed by a deposition method, and it is necessary to develop a material that can withstand long time in deposition, that is, a material having high heat resistance characteristics.

That is, in order to sufficiently exhibit the excellent characteristics of the organic electronic element, a material for forming an organic material layer in an element such as a hole injection material, a hole transport material, a light emitting material, an electron transport material, an electron injection material, an emitting-auxiliary layer material should be supported by stable and efficient materials. However, such a stable and efficient organic material layer material for an organic electronic element has not been sufficiently developed yet. Therefore, the development of new materials continues to be required, and in particular, the development of materials for the emitting-auxiliary layer is urgently required.

### DETAILED DESCRIPTION OF THE INVENTION

### Summary

In order to solve the problems of the background art described above, the present invention has revealed a compound having a novel structure, and that when the compound is applied to an organic electronic element, the luminous efficiency, stability and lifespan of the element are greatly improved.

Accordingly, it is an object of the present invention to provide a novel compound, an organic electronic element using the same, and an electronic device thereof.

### Technical Solution

The present invention provides a compound represented by Formula 1.

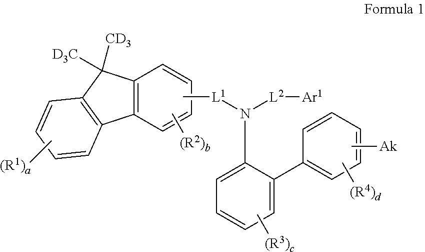

In another aspect, the present invention provides an organic electronic element comprising a compound represented by Formula 1 and an electronic device thereof.

### Effects of the Invention

By using the compound according to the present invention, it is possible to achieve a high luminous efficiency, a low driving voltage, and a high heat resistance of the element, and can greatly improve the color purity and lifespan of the element.

### BRIEF DESCRIPTION OF THE DRAWINGS

FIG. 1 to FIG. 3 illustrate an example of an organic electronic element according to the present invention.

FIG. 4 shows a Formula according to one aspect of the present invention.

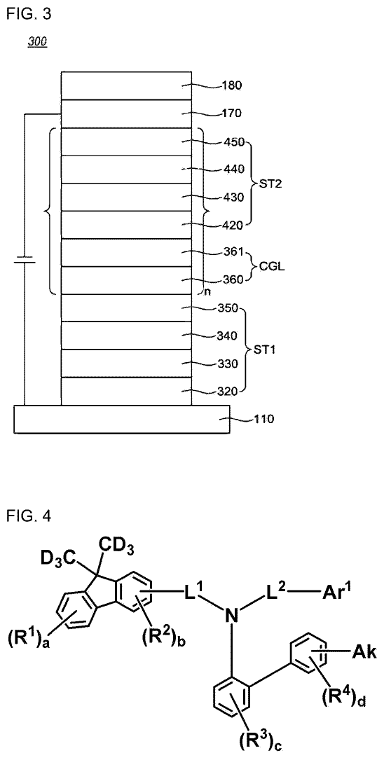

### DESCRIPTION OF THE NUMERALS IN THE DRAWINGS

| | |
|---|---|
| 100, 200, 300: organic | 110: the first electrode |
| electronic element | |
| 120: hole injection layer | 130: hole transport layer |
| 140: emitting layer | 150: electron transport layer |
| 160: electron injection layer | 170: second electrode |
| 180: light efficiency | 210: buffer layer |
| enhancing Layer | |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine (F), bromine (Br), chlorine (Cl), or iodine (I).

Unless otherwise stated, the term “alkyl” or “alkyl group”, as used herein, has a single bond of 1 to 60 carbon atoms, 1 to 30 carbon atoms, 1 to 25 carbon atoms, 1 to 18 carbon atoms, or 1 to 12 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, 2 to 30 carbon atoms, 2 to 25 carbon atoms, 2 to 18 carbon atoms, 2 to 12 carbon atoms, but is not limited thereto, and includes a linear or a branched chain group.

Unless otherwise stated, the term “cycloalkyl”, as used herein, means alkyl forming a ring having 3 to 60 carbon atoms, 3 to 30 carbon atoms, 3 to 25 carbon atoms, 3 to 18 carbon atoms, 3 to 12 carbon atoms, but is not limited thereto.

Unless otherwise stated, the term “alkoxyl group”, “alkoxy group” or “alkyloxy group”, as used herein, means an alkyl group bonded to oxygen radical, but is not limited thereto, and has 1 to 60 carbon atoms, 1 to 30 carbon atoms, 1 to 25 carbon atoms, 1 to 18 carbon atoms, or 1 to 12 carbon atoms.

Unless otherwise stated, the term “aryloxyl group” or “aryloxy group”, as used herein, means an aryl group bonded to oxygen radical, but is not limited thereto, and has 6 to 60 carbon atoms, 6 to 30 carbon atoms, 6 to 25 carbon atoms, 6 to 18 carbon atoms, or 6 to 12 carbon atoms.

Unless otherwise specified, the terms “aryl group” and “arylene group” used in the present invention have 6 to 60 carbon atoms, 6 to 30 carbon atoms, 6 to 25 carbon atoms, 6 to 18 carbon atoms, or 6 to 12 carbon atoms, respectively, but are not limited thereto. In the present invention, an aryl group or arylene group refers to an aromatic group of a single ring or multiple rings, and comprises an aromatic ring formed by combining adjacent substituents or participating in a reaction. For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, and has 2 to 60 carbon atoms, 2 to 30 carbon atoms, 2 to 25 carbon atoms, 2 to 18 carbon atoms, 2 to 12 carbon atoms, and comprises any one of a single ring or multiple ring, and may include heteroaliphatic ring and heteroaromatic ring. Also, the heterocyclic group may also be formed in conjunction with an adjacent group.

Unless otherwise stated, the term “heteroatom”, as used herein, represents at least one of N, O, S, P, or Si.

Additionally, a “heterocyclic group” may also comprise a ring including SO₂ instead of ring-forming carbon. For example, a “heterocyclic group” includes the following compound.

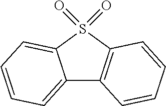 [SMILES: O=S1(=O)c2ccccc2-c2ccccc21]

Unless otherwise stated, the term “fluorenyl group”, “fluorenylene group” or “fluorentriyl group” as used herein, means a monovalent or divalent functional group, in which R, R′ and R″ are all hydrogen in the following structures, and the term “substituted fluorenyl group” or “substituted fluorenylene group” means that at least one of the substituents R, R′ and R″ is a substituent other than hydrogen, and comprise those in which R and R′ are bonded to each other to form a spiro compound together with the carbon to which they are bonded.

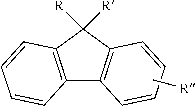

The term “spiro compound”, as used herein, has a ‘spiro union’, and a spiro union means a connection formed by 2 rings sharing only one atom. Wherein, the atoms shared between the 2 rings are called ‘spiro atoms’, and depending on the number of spiro atoms contained in a compound, they are called ‘monospiro-’, ‘dispiro-’, and ‘trispiro-’ compounds, respectively.

Unless otherwise stated, the term “aliphatic” as used herein means an aliphatic hydrocarbon having 1 to 60 carbon atoms, 1 to 30 carbon atoms, 1 to 25 carbon atoms, 1 to 18 carbon atoms or 1 to 12 carbon atoms, and “aliphatic ring” means an aliphatic hydrocarbon ring having 3 to 60 carbon atoms, 3 to 30 carbon atoms, 3 to 25 carbon atoms, 3 to 18 carbon atoms or 3 to 12 carbon atoms.

Unless otherwise stated, the term “ring”, as used herein, means an aliphatic ring having 3 to 60 carbon atoms, 3 to 30 carbon atoms, 3 to 25 carbon atoms, 3 to 18 carbon atoms or 3 to 12 carbon atoms; or an aromatic ring having 6 to 60 carbon atoms, 6 to 30 carbon atoms, 6 to 25 carbon atoms, 6 to 18 carbon atoms, or 6 to 12 carbon atoms; or a heterocyclic having 2 to 60 carbon atoms, 2 to 30 carbon atoms, 2 to 25 carbon atoms, 2 to 18 carbon atoms, 2 to 12 carbon atoms, or a fused ring formed by the combination thereof, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

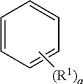

Here, when a is an integer of 0, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

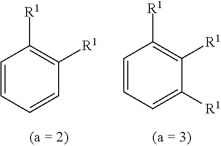

Hereinafter, a compound according to one aspect of the present invention and an organic electronic element comprising the same will be described.

The present invention provides a compound represented by Formula 1.

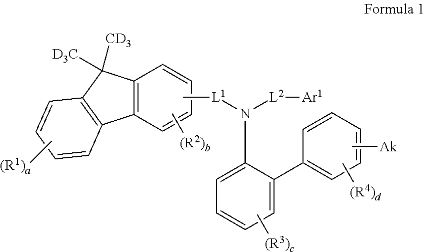

Wherein:
R¹, R², R³ and R⁴ are independently the same or different from each other and are independently selected from the group consisting of a hydrogen; deuterium; an C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and a C₃-C₃₀ aliphatic ring; or a plurality of adjacent groups may be bonded to each other to form a ring,

Wherein when R¹, R², R³ and R⁴ are an aryl group, preferably an C₆-C₆₀ aryl group, more preferably an C₆-C₂₅ aryl group, an C₆-C₁₈ aryl group or an C₆-C₁₂ aryl group, such as phenyl, biphenyl, terphenyl, naphthalene, phenanthrene, etc.

Wherein when R¹, R², R³ and R⁴ are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, naphthobenzofuran, naphthobenzothiophene, etc.

Wherein when R¹, R², R³ and R⁴ are a fused ring group, preferably a fused ring group of an C₃-C₃₀ aliphatic ring and an C₆-C₆₀ aromatic ring, more preferably a fused ring group of an C₃-C₂₅ aliphatic ring and an C₆-C₂₅ aromatic ring.

Wherein when R¹, R², R³ and R⁴ are an aliphatic ring group, preferably a C₃-C₀₀ aliphatic ring group, more preferably a C₃-C₂₅ aliphatic ring group, a C₃-C₁₈ aliphatic ring group, and a C₃-C₁₂ aliphatic ring group, and specifically, cyclobutane, cyclopentane, cyclohexane, cyclohexacene, bicycloheptane, adamantyl, etc.

Wherein when R¹, R², R³ and R⁴ are an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.

Wherein when R¹, R², R³ and R⁴ are an alkoxyl group, preferably a C₁-C₂₅ alkoxyl group.

Wherein when R¹, R², R³ and R⁴ are an aryloxy group, preferably a C₆-C₂₅ aryloxy group.

L¹ and L² are each independently selected from the group consisting of a single bond; a C₆-C₆₀ arylene group; a fluorenylene group; a C₂-C₆₀ heterocyclic group including at least one heteroatom of O, N, S, Si or P;

Wherein when L¹ and L² are an arylene group, preferably an C₆-C₆₀ arylene group, more preferably an C₆-C₂₅ arylene group, an C₆-C₁₈ arylene group or an C₆-C₁₂ arylene group, such as phenylene, biphenylene, naphthylene, terphenylene, anthracenylene, etc.

Wherein when L¹ and L² are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, benzocarbazole, naphthobenzofuran, naphthobenzothiophene, etc.

Ar¹ is an C₆-C₆₀ aryl group; a fluorenyl group; a C₂-C₆₀ heterocyclic group including at least one heteroatom of O, N, S, Si or P;

Wherein when Ar¹ is an aryl group, preferably an C₆-C₆₀ aryl group, more preferably an C₆-C₂₅ aryl group, an C₆-C₁₈ aryl group or an C₆-C₁₂ aryl group, such as phenyl, biphenyl, terphenyl, naphthalene, phenanthrene, triphenylene, etc.

Wherein when Ar¹ is a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, benzocarbazole, naphthobenzofuran, naphthobenzothiophene, etc.

Ak is a C₁˜C₅₀ alkyl group; or a C₃˜C₃₀ aliphatic ring group;

Wherein when Ak is an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.

Wherein when Ak is an aliphatic ring group, preferably a C₃-C₃₀ aliphatic ring group, more preferably a C₃-C₂₅ aliphatic ring group, a C₃-C₁₈ aliphatic ring group, and a C₃-C₁₂ aliphatic ring group, and specifically, cyclobutane, cyclopentane, cyclohexane, cyclohexacene, bicycloheptane, adamantyl, etc.
a, c and d are independently integers from 0 to 4, b is an integer from 0 to 3,
wherein the aryl group, arylene group, heterocyclic group, fluorenyl group, fluorenylene group, aliphatic ring group, fused ring group, alkyl group, alkenyl group, alkynyl group, alkoxyl group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; a C₃-C₂₀ aliphatic ring; a C₇-C₂₀ arylalkyl group; a C₈-C₂₀ arylalkenyl group; and a C₇-C₂₀ alkylaryl group; also the hydrogen of these substituents may be further substituted with one or more deuterium, and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Additionally, Formula 1 is represented by any one of the following Formulas 1-1 to 1-4.

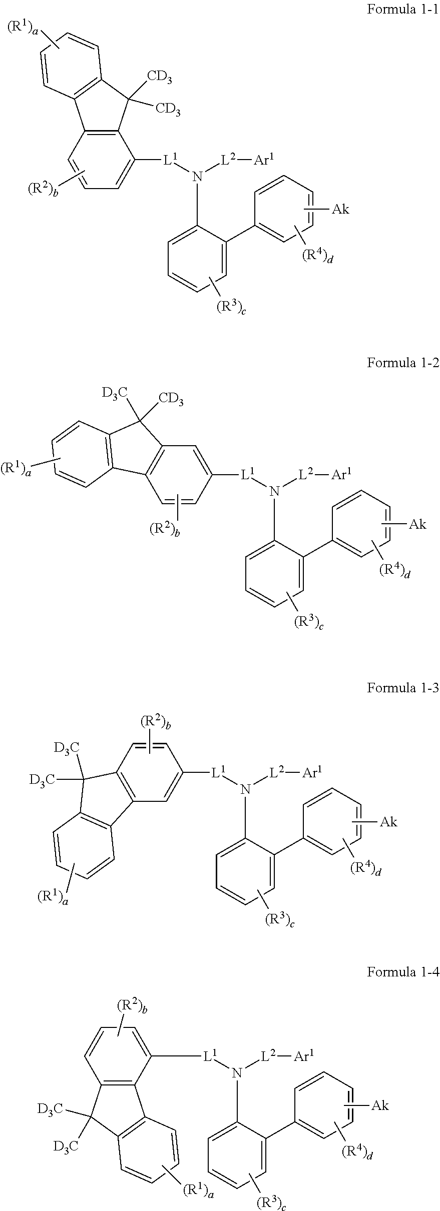

Wherein R, R², R³, R⁴, a, b, c, d, L¹, L², Ar¹ and Ak are the same as defined in Formula 1.

Additionally, Ak is represented by any one of the following Formulas Ak-1 to Ak-8.

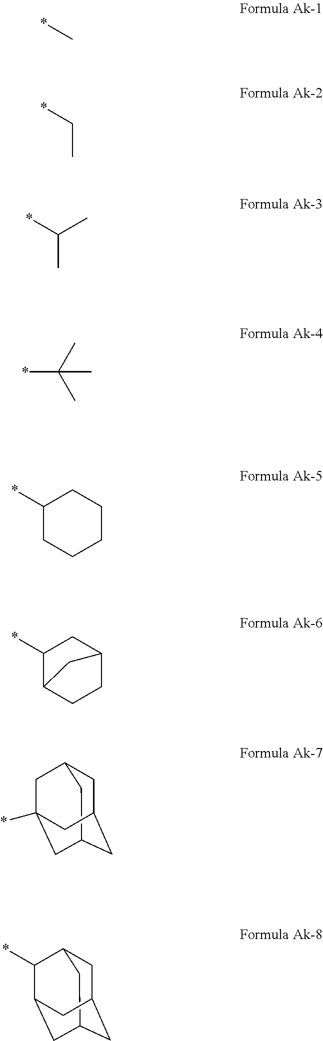

Wherein.
* indicates a position to be bonded,

Formulas Ak-1 to Ak-8 may be further substituted with one or more deuterium.

Additionally, Ar¹ is represented by any one of the following Formulas Ar-1 to Ar-11.

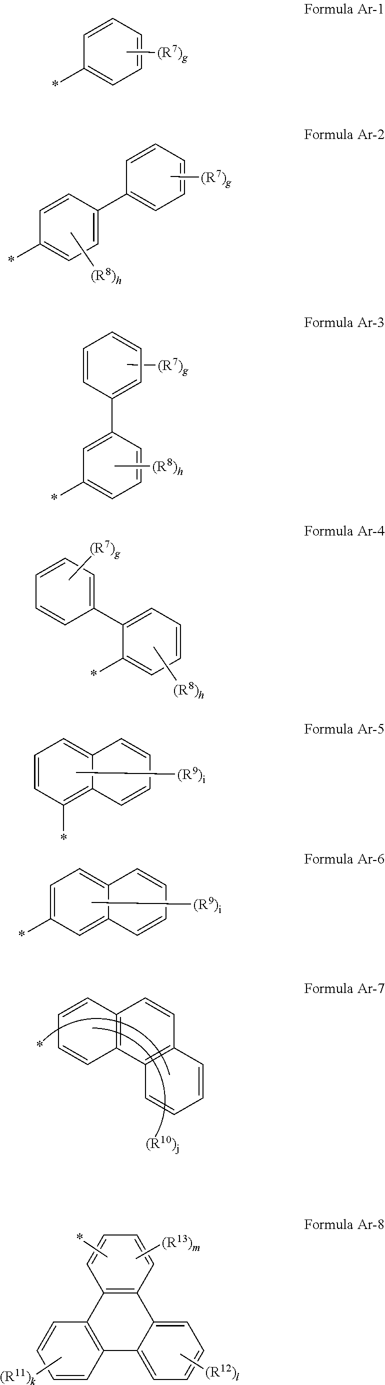

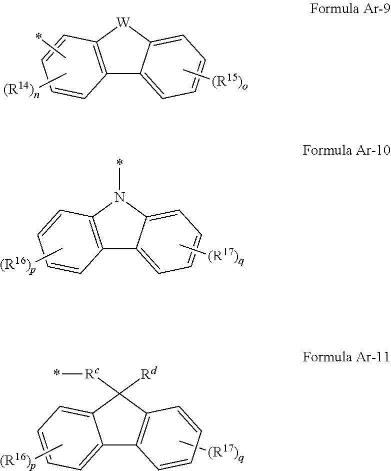

Wherein,
R⁷, R⁸, R⁹, R¹⁰, R¹¹, R¹², R¹³, R¹⁴, R¹⁵, R¹⁶ and R¹⁷ are independently the same or different from each other and are independently selected from the group consisting of a hydrogen; or deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ aliphatic group; C₇-C₂₀ arylalkyl group; C₈-C₂₀ arylalkenyl group; and a C₇-C₂₀ alkylaryl group; or a plurality of adjacent groups may be bonded to each other to form a ring,
R^{c} is selected from a group consisting of a C₁-C₆₀ alkyl group; an C₆-C₆₀ aryl group; and a C₂-C₆₀ heterocyclic group including at least one heteroatom of O, N, S, Si or P;
R^{d} is selected from a group consisting of a hydrogen; or deuterium; a C₁-C₅₀ alkyl group; an C₆-C₆₀ aryl group; and a C₂-C₆₀ heterocyclic group including at least one heteroatom of O, N, S, Si or P;

Wherein R^{c} and R^{d} are an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.

Wherein R^{c} and R^{d} are an aryl group, preferably an C₆-C₃₀ aryl group, more preferably an C₆-C₂₅ aryl group, an C₆-C₁₈ aryl group or an C₆-C₁₂ aryl group, such as phenyl, biphenyl, terphenyl, naphthalene, phenanthrene, triphenylene, etc.

Wherein R^{c} and R^{d} are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, benzocarbazole, naphthobenzofuran, naphthobenzothiophene, etc.

R^{c} and R^{d} can be bonded to each other to form a spiro, g is an integer from 0 to 5, h, k, l, o, p and q are independently integers from 0 to 4, i is an integer from 0 to 7, j is an integer from 0 to 9, m and n are independently integers from 0 to 3,
W is O, S, C(R²⁸)(R²⁹) or NR³⁰,
R²⁸, R²⁹ and R³⁰ are selected from a group consisting of a C₁-C₂₀ alkyl group; a C₂-C₂₀ alkenyl group; C₆-C₂₀ aryl group; C₆-C₂₀ aryl group substituted with deuterium; a fluorenyl group; a C₂-C₂₀ heterocyclic group including at least one heteroatom of O, N, S, Si or P; C₃-C₂₀ aliphatic group; C₇-C₂₀ arylalkyl group; C₈-C₂₀ arylalkenyl group; and a C₇-C₂₀ alkylaryl group; or, R²⁷ and R²⁸ may be bonded to each other to form a ring,
* indicates a position to be bonded,
L¹ and L² are selected from a single bond or one of the following Formulae L-1 to L-27.

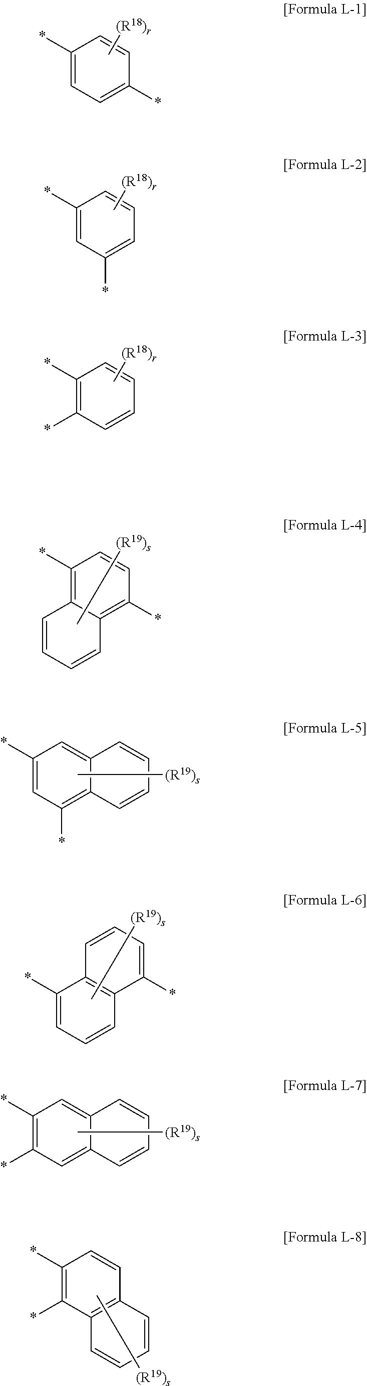

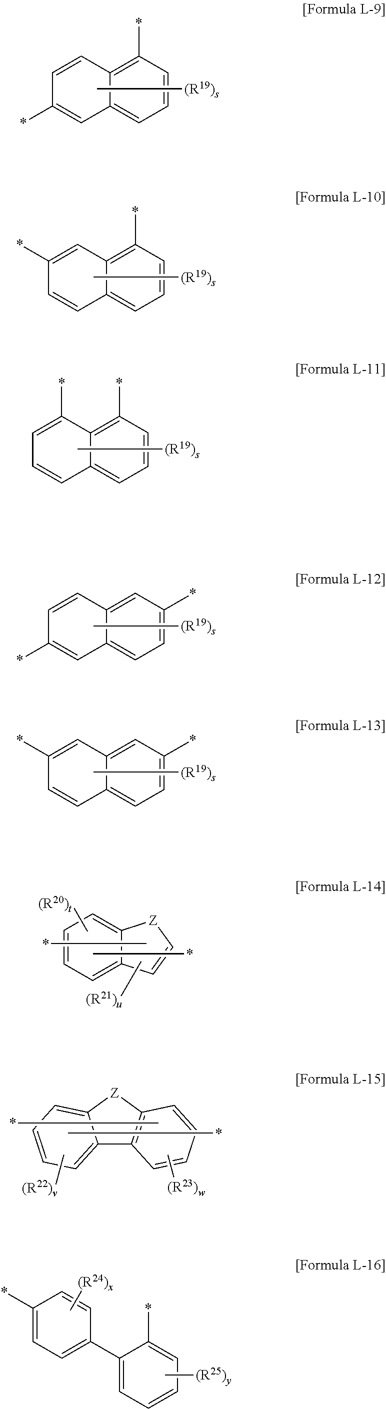

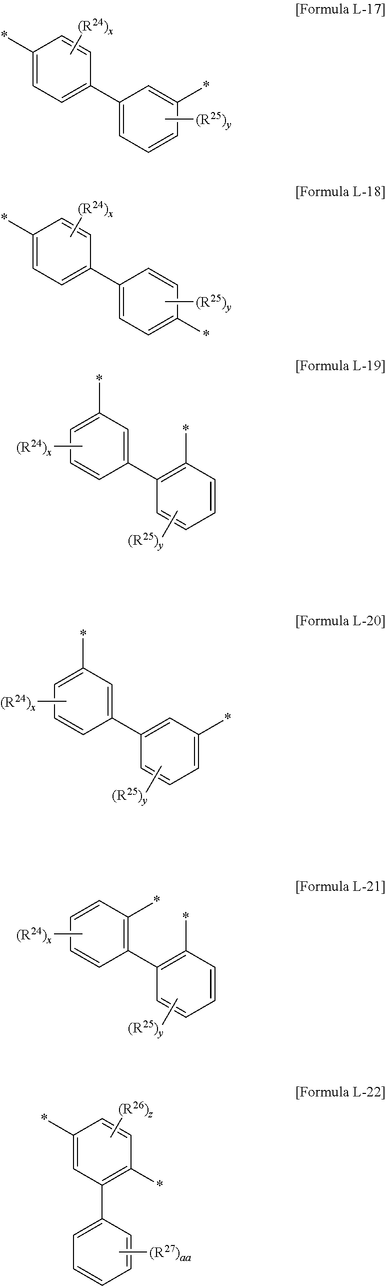

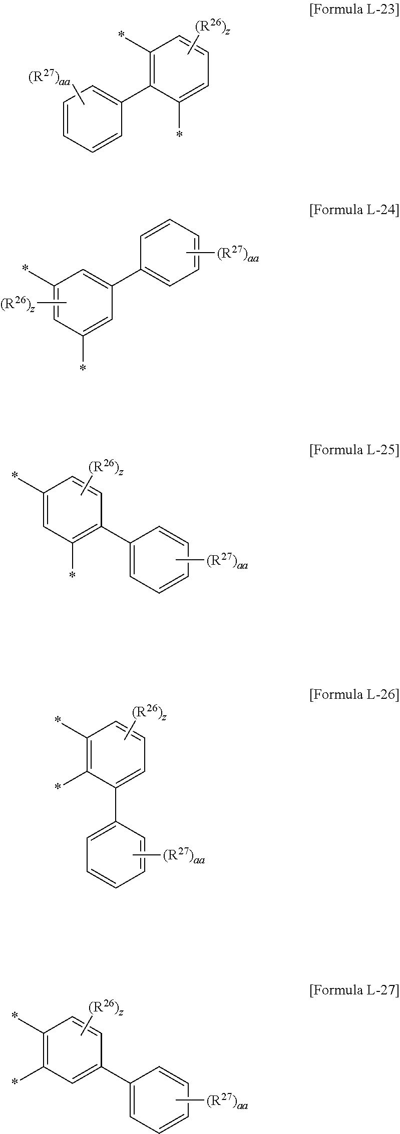

Wherein,
Z is O, S, C(R³¹)(R³²) or N—Ar⁵,
R¹⁸, R¹⁹, R²⁰, R²¹, R²², R²³, R²⁴, R²⁵, R²⁶ and R²⁷ are the same as definition of R⁷, or a plurality of adjacent groups may be bonded to each other to form a ring,
R³¹ and R³² are the same as the definition of R²⁸,
Ar⁵ is selected from a group consisting of a C₁-C₂₀ alkyl group; C₆-C₂₀ aryl group; C₆-C₂₀ aryl group substituted with deuterium; a fluorenyl group; a C₂-C₂₀ heterocyclic group including at least one heteroatom of O, N, S, Si or P;
r, t, v, w, x, y and z are independently integers from 0 to 4, s is an integer from 0 to 6, u is an integer from 0 to 2, aa is an integer from 0 to 5,
* indicates a position to be bonded.

Ar¹ is represented by the following Formula Ar-12 or Formula Ar-13.

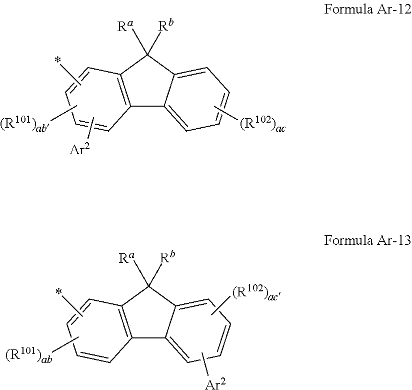

Wherein,
R^{a} and R^{b} are independently selected from the group consisting of C₁-C₂₀ alkyl group; C₁-C₂₀ alkyl group substituted with deuterium; C₂-C₂₀ alkenyl group; C₆-C₂₀ aryl group; C₆-C₂₀ aryl group substituted with deuterium; a fluorenyl group; a C₂˜C₂₀ heterocyclic group including at least one heteroatom of O, N, S, Si or P; C₃-C₂₀ aliphatic group; C₇-C₂₀ arylalkyl group; a C₈-C₂₀ arylalkenyl group; and a C₇-C₂₀ alkylaryl group; or R^{a} and R^{b} can be bonded to each other to form a spiro,
R^{a} and R^{b} may preferably be methyl, deuterated methyl, phenyl or deuterated phenyl, more preferably R^{a} and R^{b} may be the same as each other, and more preferably R^{a} and R^{b} may be methyl or deuterated methyl at the same time.

R¹⁰¹ and R¹⁰² are the same as the definition of R⁷, or adjacent groups may be bonded to each other to form a ring,
Ar² is selected from the group consisting of a C₁-C₂₀ alkyl group substituted or unsubstituted with deuterium; a C₆-C₂₀ aryl group substituted or unsubstituted with deuterium; a C₂˜C₂₀ heterocyclic group including at least one heteroatom of O, N, S, Si or P; and a C₇-C₂₀ alkylaryl group;
Ar² may preferably be methyl, deuterated methyl, phenyl, deuterated phenyl or phenyl substituted with an alkyl group, and more preferably may be phenyl, deuterated phenyl or phenyl substituted with an alkyl group.

ab′ is an integer from 0 to 2, ac is an integer from 0 to 4, and ab and ac′ are independently integers from 0 to 3,
* indicates a position to be bonded.

Formula Ar-12 may preferably be any one of the following Formulas Ar-12-1 to Ar-12-12.

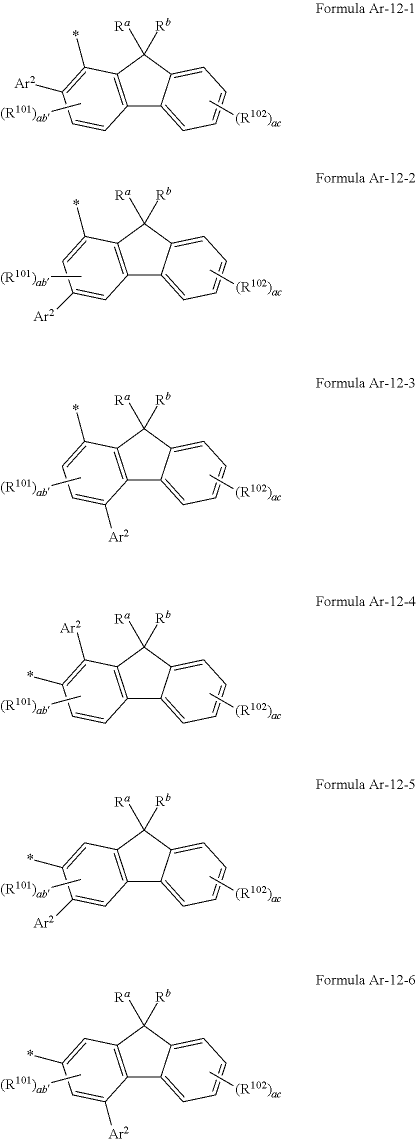

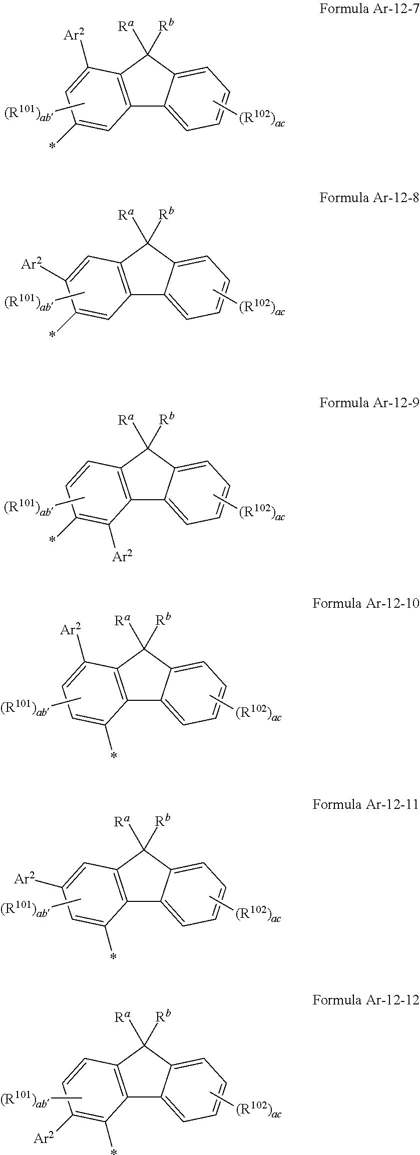

Wherein:
Ar², R^{a}, R^{b}, R¹⁰¹, R¹⁰², ab′, ac and * are the same as defined in Formula Ar-12.

Formula Ar-13 may preferably be any one of the following Formulas Ar-13-1 to Ar-13-16.

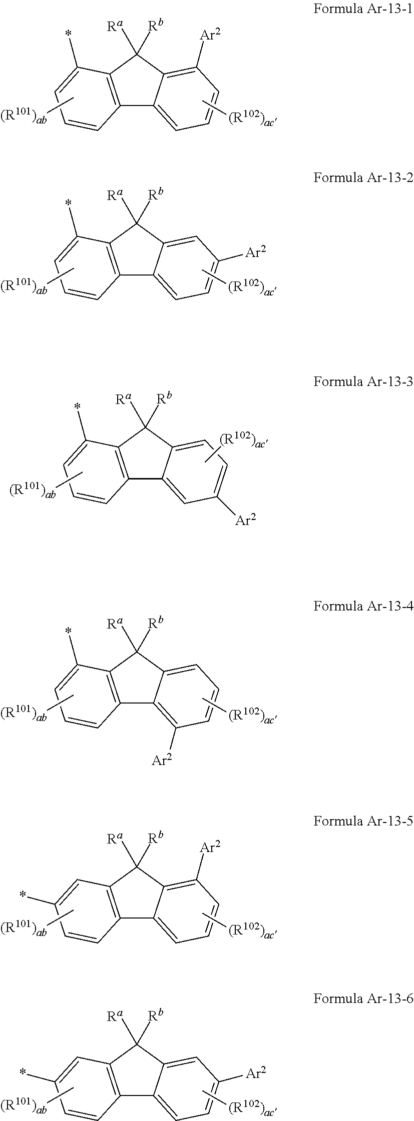

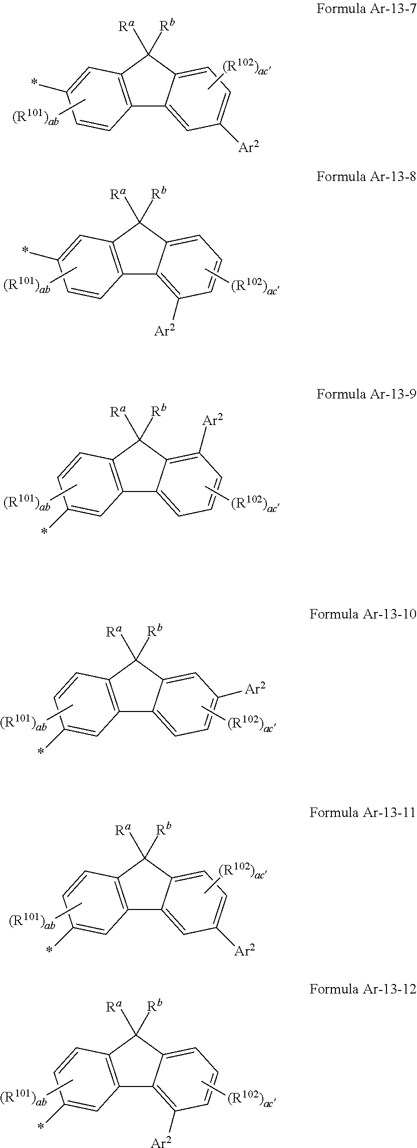

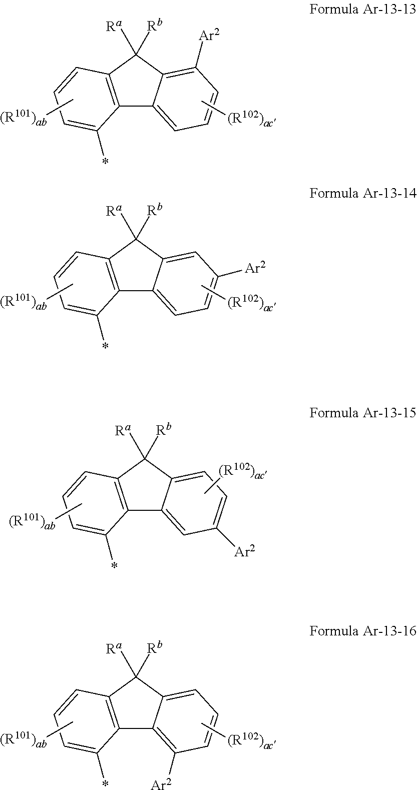

Wherein:
Ar², R^{a}, R^{b}, R¹⁰¹, R¹⁰², ab, ac′ and * are the same as defined in Formula Ar-13.

Specifically, the compound represented by Formula 1 may be any one of the following compounds P-1 to P-160, but is not limited thereto.

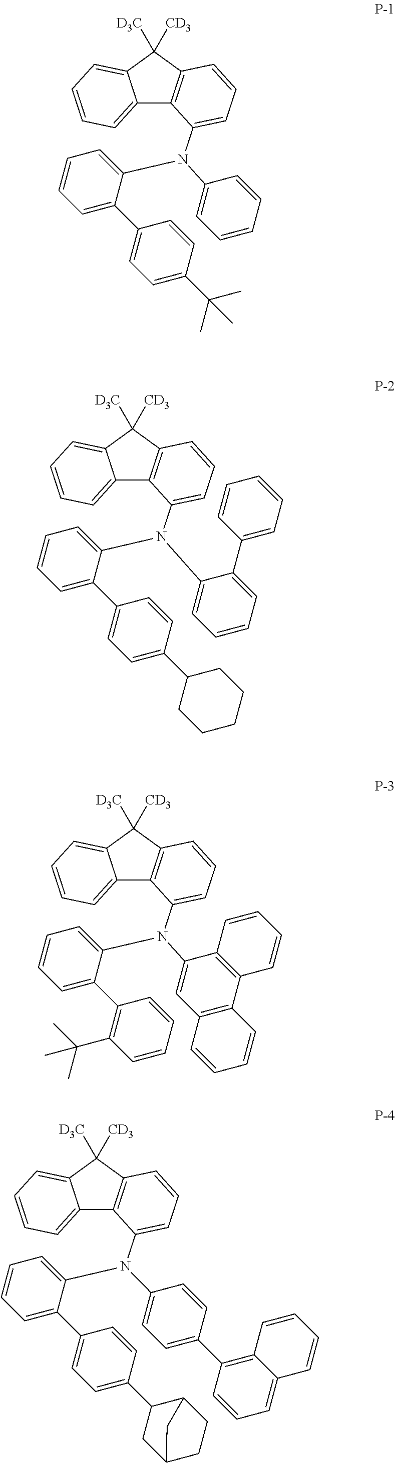 [SMILES: [2H]C([2H])([2H])C1(C([2H])([2H])[2H])c2ccccc2-c2c(N(c3ccccc3)c3ccccc3-c3ccc(C(C)(C)C)cc3)cccc21 ; [2H]C([2H])([2H])C1(C([2H])([2H])[2H])c2ccccc2-c2c(N(c3ccccc3-c3ccccc3)c3ccccc3-c3ccc(C4CCCCC4)cc3)cccc21 ; [2H]C([2H])([2H])C1(C([2H])([2H])[2H])c2ccccc2-c2c(N(c3ccccc3-c3ccccc3C(C)(C)C)c3cc4ccccc4c4ccccc34)cccc21 ; [2H]C([2H])([2H])C1(C([2H])([2H])[2H])c2ccccc2-c2c(N(c3ccc(-c4cccc5ccccc45)cc3)c3ccccc3-c3ccc(C4CC5CCC4C5)cc3)cccc21]

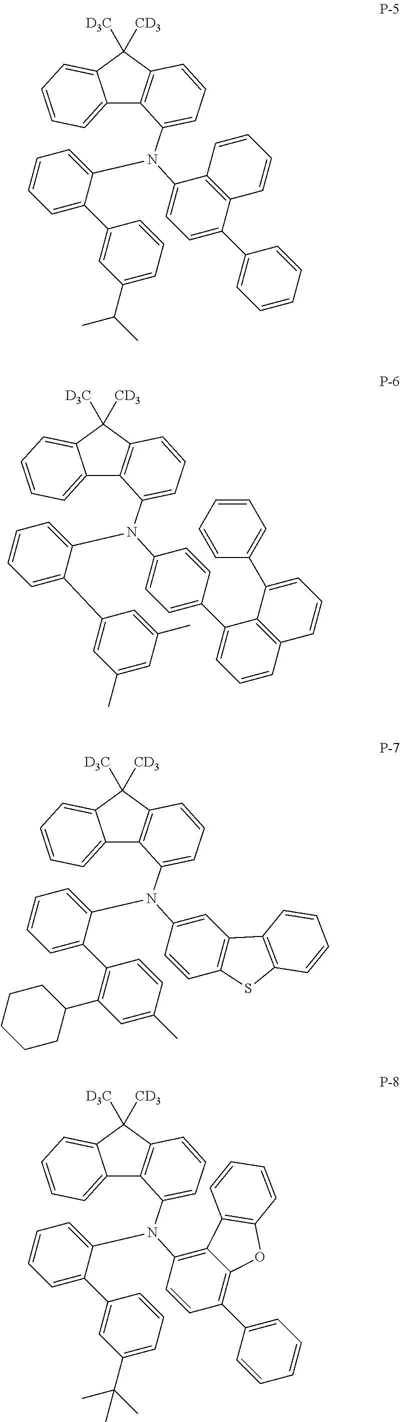 [SMILES: [2H]C([2H])([2H])C1(C([2H])([2H])[2H])c2ccccc2-c2c(N(c3ccccc3-c3cccc(C(C)C)c3)c3ccc(-c4ccccc4)c4ccccc34)cccc21 ; [2H]C([2H])([2H])C1(C([2H])([2H])[2H])c2ccccc2-c2c(N(c3ccc(-c4cccc5cccc(-c6ccccc6)c45)cc3)c3ccccc3-c3cc(C)cc(C)c3)cccc21 ; [2H]C([2H])([2H])C1(C([2H])([2H])[2H])c2ccccc2-c2c(N(c3ccc4sc5ccccc5c4c3)c3ccccc3-c3ccc(C)cc3C3CCCCC3)cccc21 ; [2H]C([2H])([2H])C1(C([2H])([2H])[2H])c2ccccc2-c2c(N(c3ccccc3-c3cccc(C(C)(C)C)c3)c3ccc(-c4ccccc4)c4oc5ccccc5c34)cccc21]

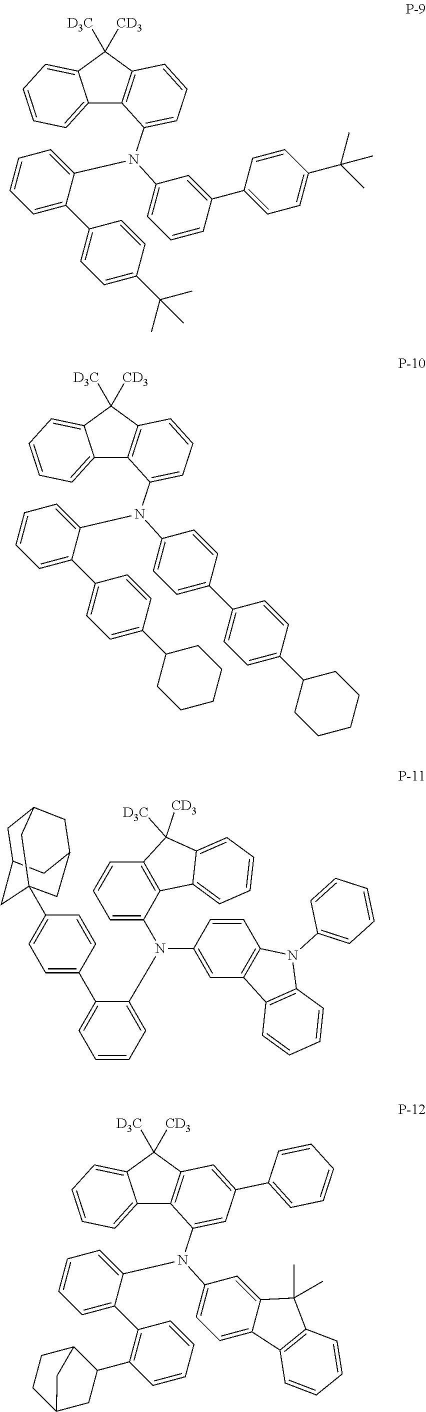

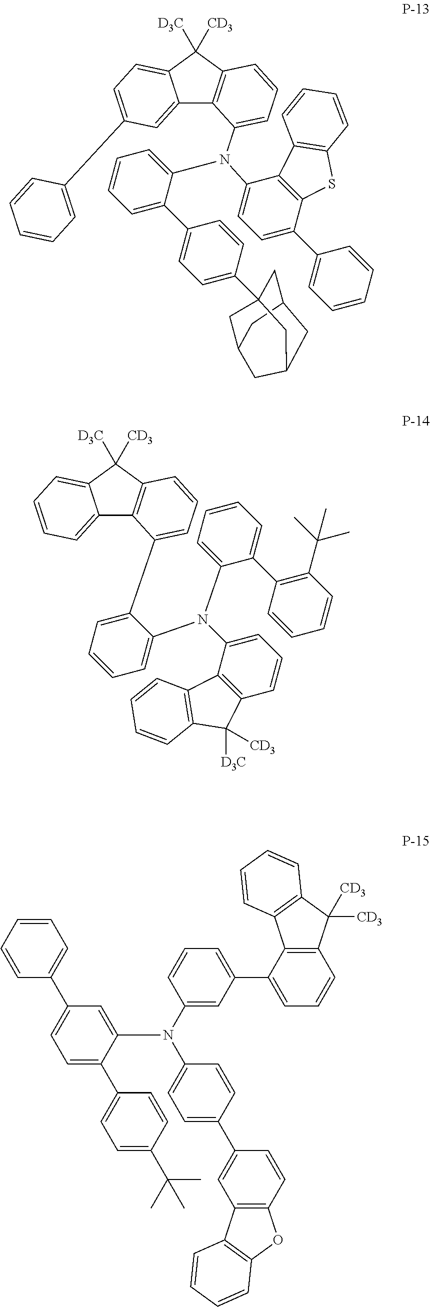 [SMILES: [2H]C([2H])([2H])C1(C([2H])([2H])[2H])c2ccc(-c3ccccc3)cc2-c2c(N(c3ccccc3-c3ccc(C45CC6CC(CC(C6)C4)C5)cc3)c3ccc(-c4ccccc4)c4sc5ccccc5c34)cccc21 ; [2H]C([2H])([2H])C1(C([2H])([2H])[2H])c2ccccc2-c2c(-c3ccccc3N(c3ccccc3-c3ccccc3C(C)(C)C)c3cccc4c3-c3ccccc3C4(C([2H])([2H])[2H])C([2H])([2H])[2H])cccc21 ; [2H]C([2H])([2H])C1(C([2H])([2H])[2H])c2ccccc2-c2c(-c3cccc(N(c4ccc(-c5ccc6oc7ccccc7c6c5)cc4)c4cc(-c5ccccc5)ccc4-c4ccc(C(C)(C)C)cc4)c3)cccc21]

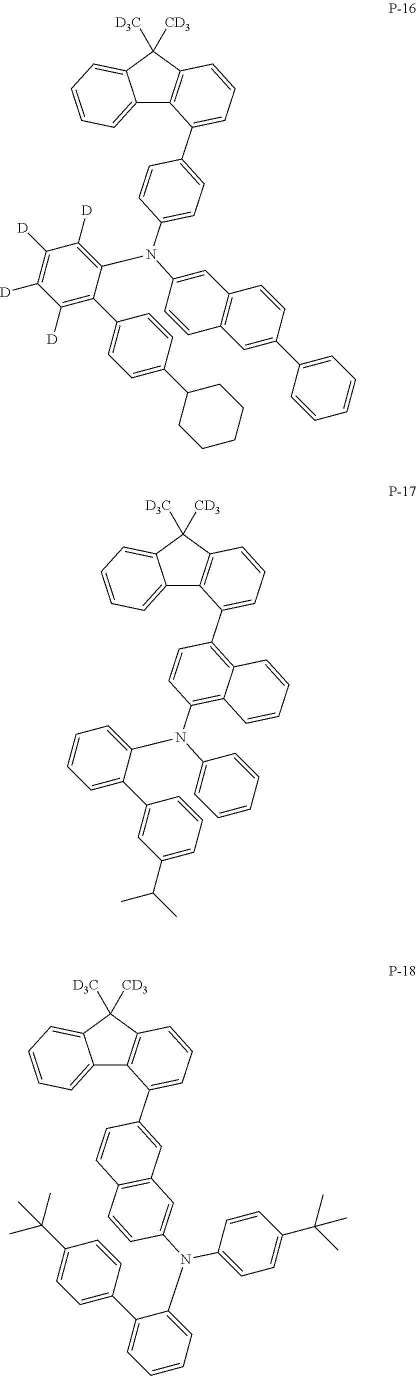 [SMILES: [2H]c1c([2H])c([2H])c(N(c2ccc(-c3cccc4c3-c3ccccc3C4(C([2H])([2H])[2H])C([2H])([2H])[2H])cc2)c2ccc3cc(-c4ccccc4)ccc3c2)c(-c2ccc(C3CCCCC3)cc2)c1[2H] ; [2H]C([2H])([2H])C1(C([2H])([2H])[2H])c2ccccc2-c2c(-c3ccc(N(c4ccccc4)c4ccccc4-c4cccc(C(C)C)c4)c4ccccc34)cccc21 ; [2H]C([2H])([2H])C1(C([2H])([2H])[2H])c2ccccc2-c2c(-c3ccc4ccc(N(c5ccc(C(C)(C)C)cc5)c5ccccc5-c5ccc(C(C)(C)C)cc5)cc4c3)cccc21]

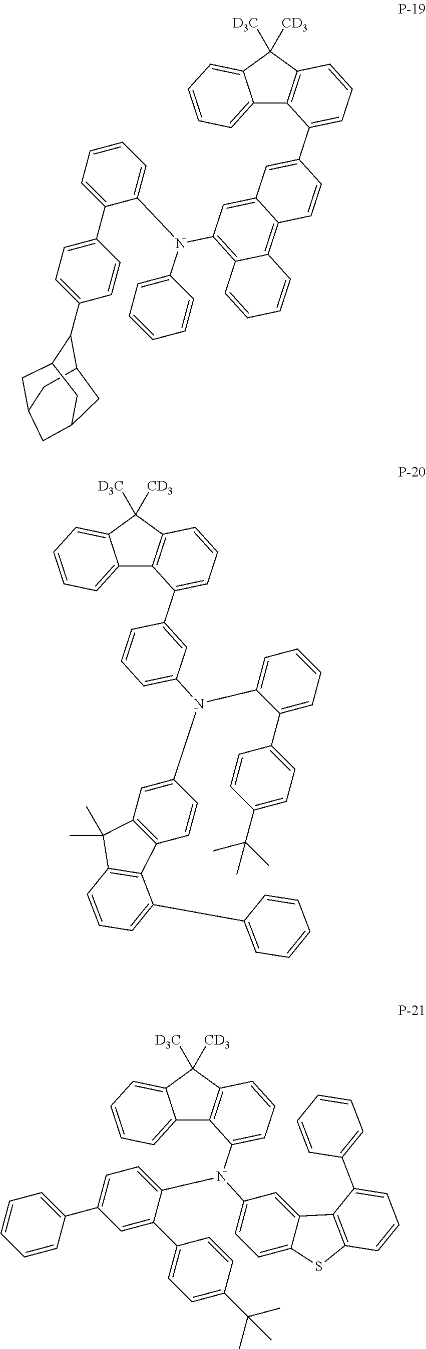 [SMILES: [2H]C([2H])([2H])C1(C([2H])([2H])[2H])c2ccccc2-c2c(-c3ccc4c(c3)cc(N(c3ccccc3)c3ccccc3-c3ccc(C5C6CC7CC(C6)CC5C7)cc3)c3ccccc34)cccc21 ; [2H]C([2H])([2H])C1(C([2H])([2H])[2H])c2ccccc2-c2c(-c3cccc(N(c4ccc5c(c4)C(C)(C)c4cccc(-c6ccccc6)c4-5)c4ccccc4-c4ccc(C(C)(C)C)cc4)c3)cccc21 ; [2H]C([2H])([2H])C1(C([2H])([2H])[2H])c2ccccc2-c2c(N(c3ccc4sc5cccc(-c6ccccc6)c5c4c3)c3ccc(-c4ccccc4)cc3-c3ccc(C(C)(C)C)cc3)cccc21]

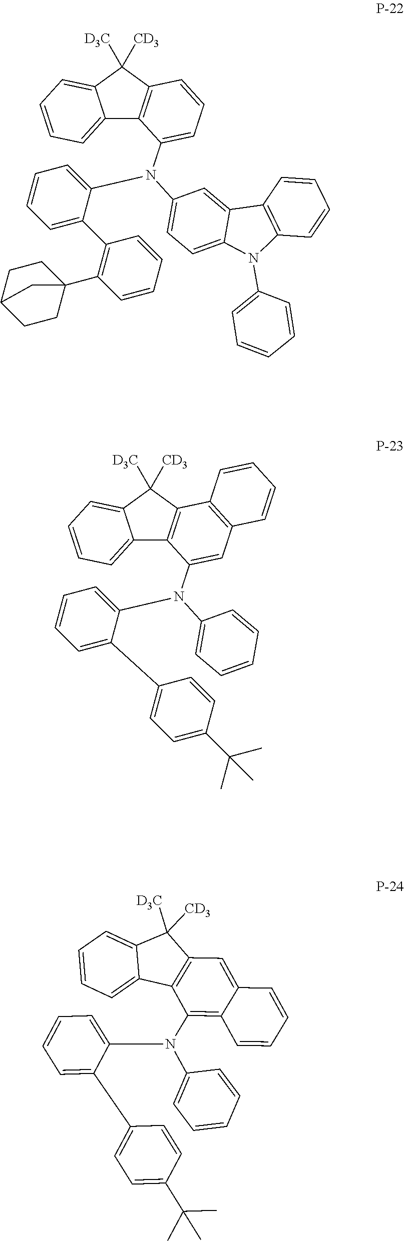 [SMILES: [2H]C([2H])([2H])C1(C([2H])([2H])[2H])c2ccccc2-c2c(N(c3ccc4c(c3)c3ccccc3n4-c3ccccc3)c3ccccc3-c3ccccc3C34CCC(CC3)C4)cccc21 ; [2H]C([2H])([2H])C1(C([2H])([2H])[2H])c2ccccc2-c2c(N(c3ccccc3)c3ccccc3-c3ccc(C(C)(C)C)cc3)cc3ccccc3c21 ; [2H]C([2H])([2H])C1(C([2H])([2H])[2H])c2ccccc2-c2c1cc1ccccc1c2N(c1ccccc1)c1ccccc1-c1ccc(C(C)(C)C)cc1]

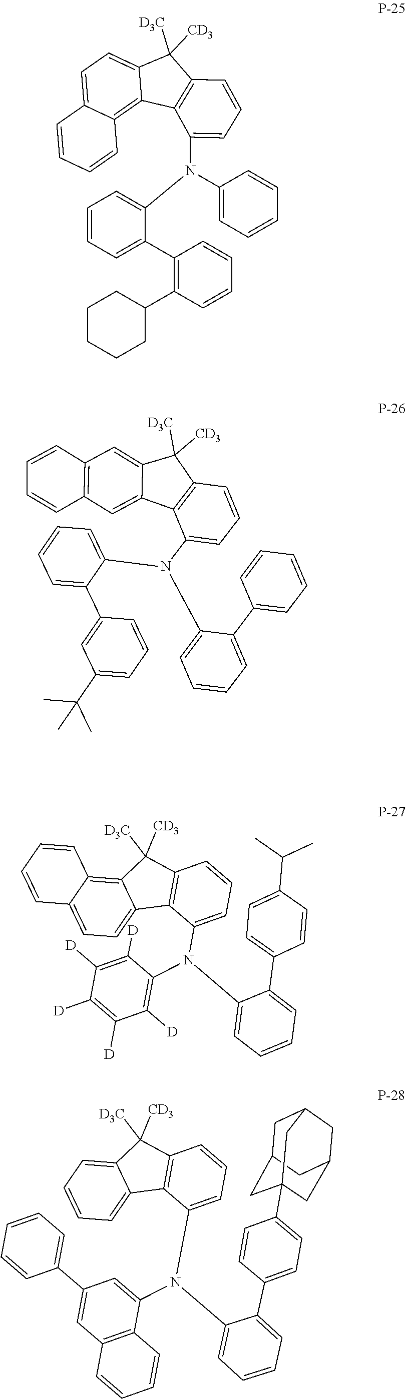 [SMILES: [2H]C([2H])([2H])C1(C([2H])([2H])[2H])c2cccc(N(c3ccccc3)c3ccccc3-c3ccccc3C3CCCCC3)c2-c2c1ccc1ccccc21 ; [2H]C([2H])([2H])C1(C([2H])([2H])[2H])c2cc3ccccc3cc2-c2c(N(c3ccccc3-c3ccccc3)c3ccccc3-c3cccc(C(C)(C)C)c3)cccc21 ; [2H]c1c([2H])c([2H])c(N(c2ccccc2-c2ccc(C(C)C)cc2)c2cccc3c2-c2ccc4ccccc4c2C3(C([2H])([2H])[2H])C([2H])([2H])[2H])c([2H])c1[2H] ; [2H]C([2H])([2H])C1(C([2H])([2H])[2H])c2ccccc2-c2c(N(c3ccccc3-c3ccc(C45CC6CC(CC(C6)C4)C5)cc3)c3cc(-c4ccccc4)cc4ccccc34)cccc21]

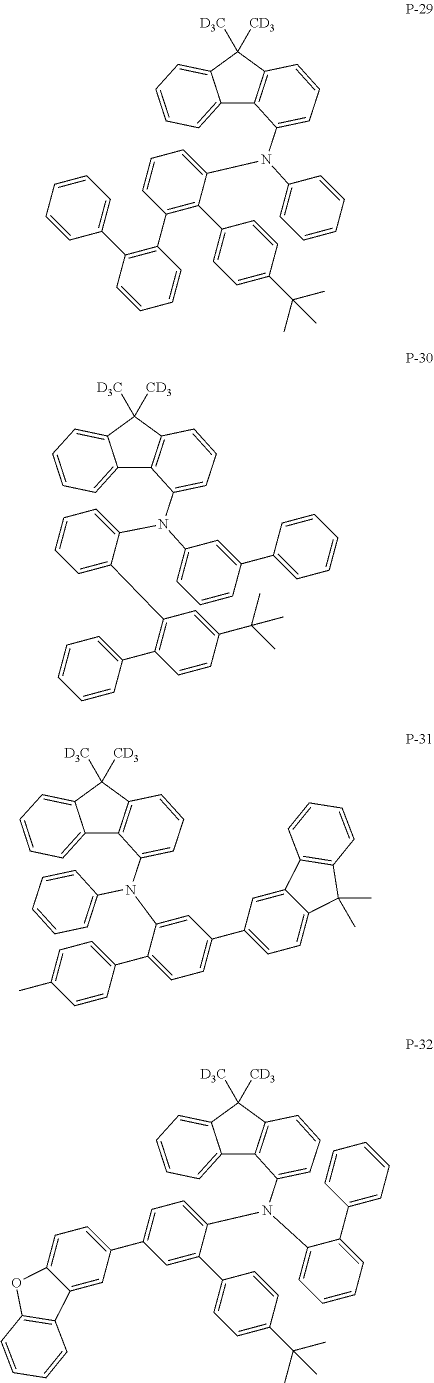

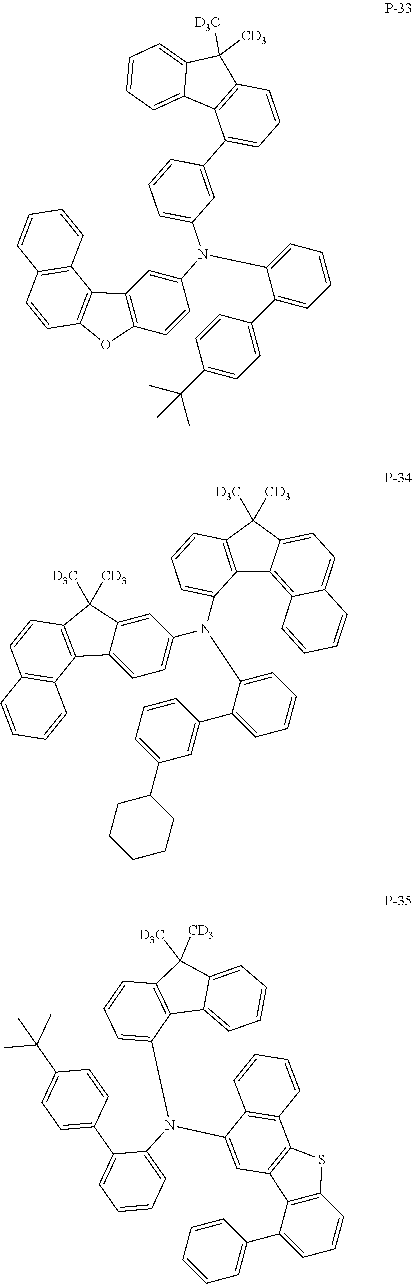 [SMILES: [2H]C([2H])([2H])C1(C([2H])([2H])[2H])c2ccccc2-c2c(-c3cccc(N(c4ccc5oc6ccc7ccccc7c6c5c4)c4ccccc4-c4ccc(C(C)(C)C)cc4)c3)cccc21 ; [2H]C([2H])([2H])C1(C([2H])([2H])[2H])c2cc(N(c3ccccc3-c3cccc(C4CCCCC4)c3)c3cccc4c3-c3c(ccc5ccccc35)C4(C([2H])([2H])[2H])C([2H])([2H])[2H])ccc2-c2c1ccc1ccccc21 ; [2H]C([2H])([2H])C1(C([2H])([2H])[2H])c2ccccc2-c2c(N(c3ccccc3-c3ccc(C(C)(C)C)cc3)c3cc4c(sc5cccc(-c6ccccc6)c54)c4ccccc34)cccc21]

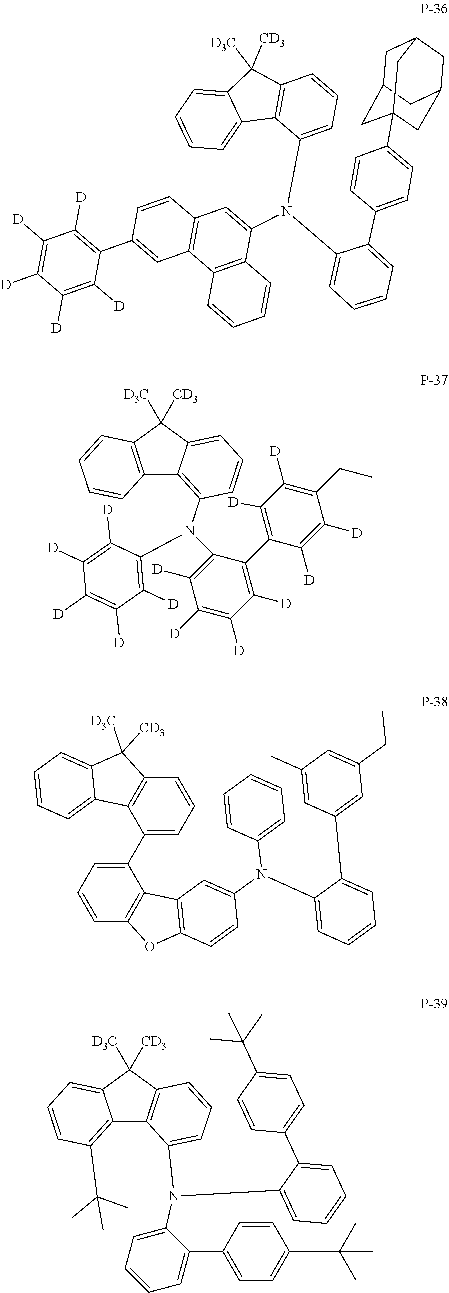

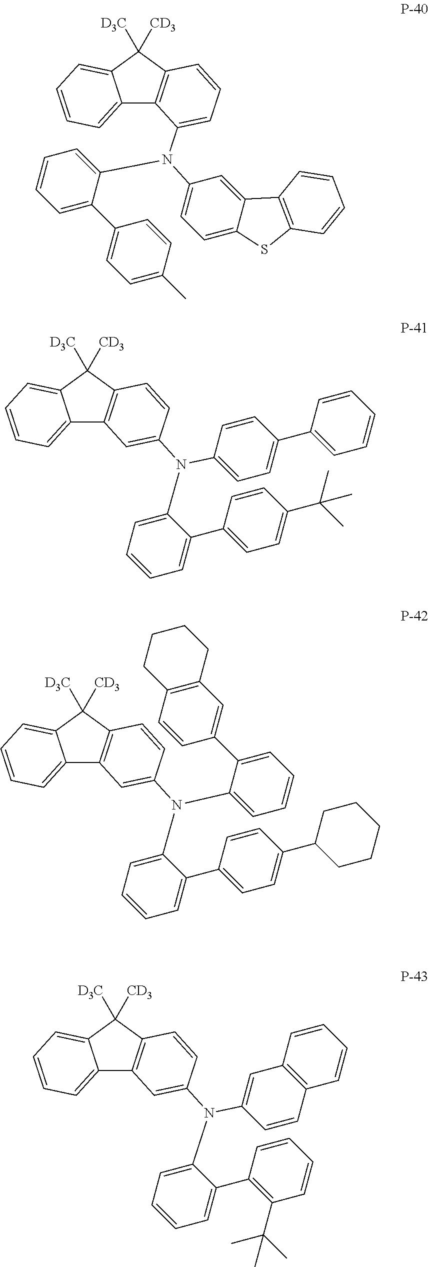 [SMILES: [2H]C([2H])([2H])C1(C([2H])([2H])[2H])c2ccccc2-c2c(N(c3ccc4sc5ccccc5c4c3)c3ccccc3-c3ccc(C)cc3)cccc21 ; [2H]C([2H])([2H])C1(C([2H])([2H])[2H])c2ccccc2-c2cc(N(c3ccc(-c4ccccc4)cc3)c3ccccc3-c3ccc(C(C)(C)C)cc3)ccc21 ; [2H]C([2H])([2H])C1(C([2H])([2H])[2H])c2ccccc2-c2cc(N(c3ccccc3-c3ccc(C4CCCCC4)cc3)c3ccccc3-c3ccc4c(c3)CCCC4)ccc21 ; [2H]C([2H])([2H])C1(C([2H])([2H])[2H])c2ccccc2-c2cc(N(c3ccc4ccccc4c3)c3ccccc3-c3ccccc3C(C)(C)C)ccc21]

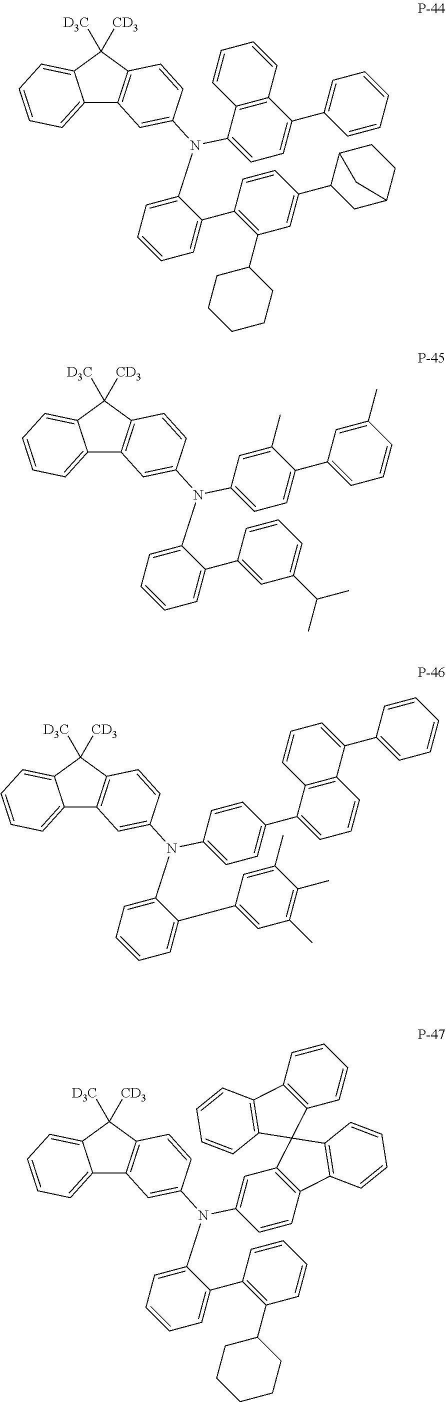 [SMILES: [2H]C([2H])([2H])C1(C([2H])([2H])[2H])c2ccccc2-c2cc(N(c3ccccc3-c3ccc(C4CC5CCC4C5)cc3C3CCCCC3)c3ccc(-c4ccccc4)c4ccccc34)ccc21 ; [2H]C([2H])([2H])C1(C([2H])([2H])[2H])c2ccccc2-c2cc(N(c3ccc(-c4cccc(C)c4)c(C)c3)c3ccccc3-c3cccc(C(C)C)c3)ccc21 ; [2H]C([2H])([2H])C1(C([2H])([2H])[2H])c2ccccc2-c2cc(N(c3ccc(-c4cccc5c(-c6ccccc6)cccc45)cc3)c3ccccc3-c3cc(C)c(C)c(C)c3)ccc21 ; [2H]C([2H])([2H])C1(C([2H])([2H])[2H])c2ccccc2-c2cc(N(c3ccc4c(c3)C3(c5ccccc5-c5ccccc53)c3ccccc3-4)c3ccccc3-c3ccccc3C3CCCCC3)ccc21]

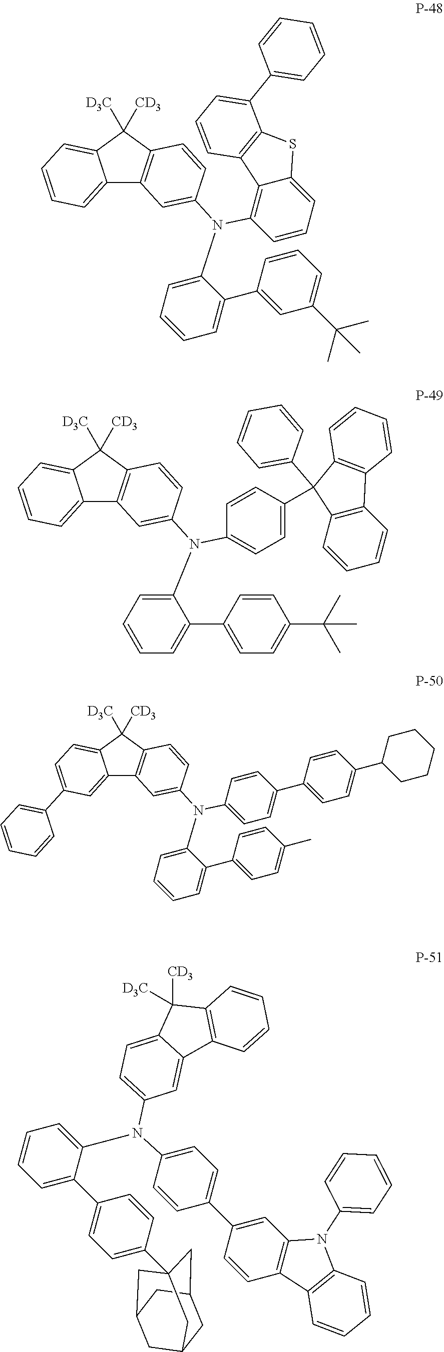

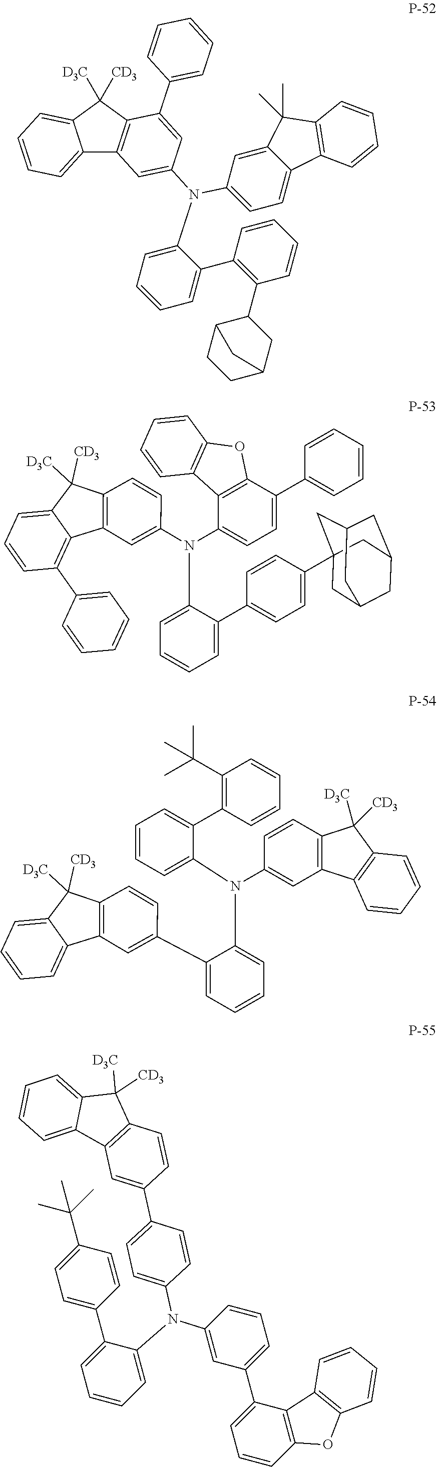

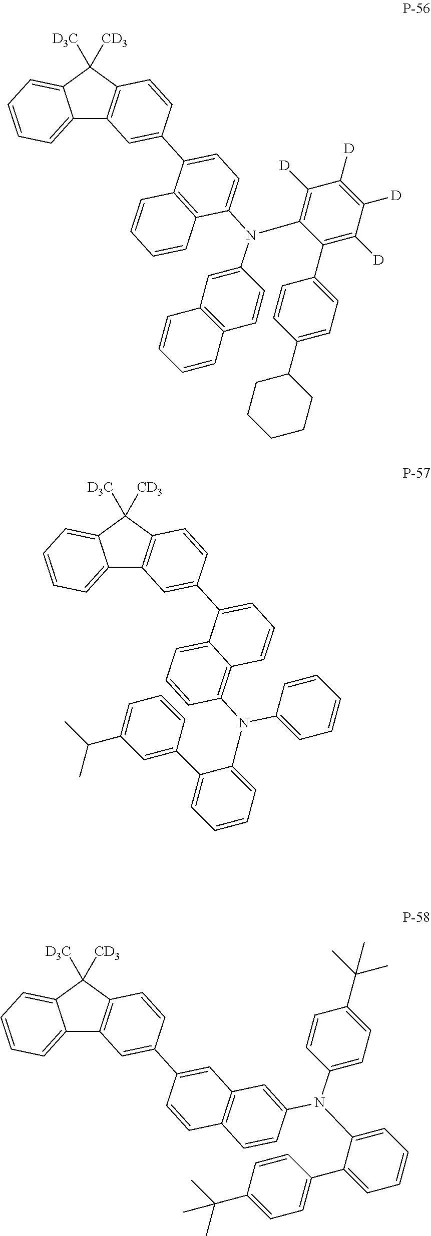 [SMILES: [2H]c1c([2H])c([2H])c(N(c2ccc3ccccc3c2)c2ccc(-c3ccc4c(c3)-c3ccccc3C4(C([2H])([2H])[2H])C([2H])([2H])[2H])c3ccccc23)c(-c2ccc(C3CCCCC3)cc2)c1[2H] ; [2H]C([2H])([2H])C1(C([2H])([2H])[2H])c2ccccc2-c2cc(-c3cccc4c(N(c5ccccc5)c5ccccc5-c5cccc(C(C)C)c5)cccc34)ccc21 ; [2H]C([2H])([2H])C1(C([2H])([2H])[2H])c2ccccc2-c2cc(-c3ccc4ccc(N(c5ccc(C(C)(C)C)cc5)c5ccccc5-c5ccc(C(C)(C)C)cc5)cc4c3)ccc21]

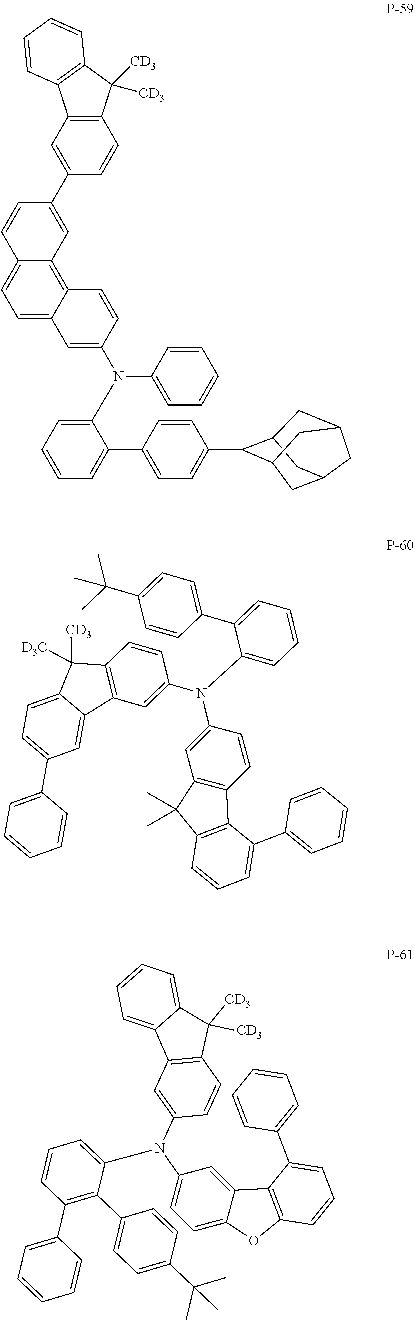 [SMILES: [2H]C([2H])([2H])C1(C([2H])([2H])[2H])c2ccccc2-c2cc(-c3ccc4ccc5cc(N(c6ccccc6)c6ccccc6-c6ccc(C7C8CC9CC(C8)CC7C9)cc6)ccc5c4c3)ccc21 ; [2H]C([2H])([2H])C1(C([2H])([2H])[2H])c2ccc(-c3ccccc3)cc2-c2cc(N(c3ccc4c(c3)C(C)(C)c3cccc(-c5ccccc5)c3-4)c3ccccc3-c3ccc(C(C)(C)C)cc3)ccc21 ; [2H]C([2H])([2H])C1(C([2H])([2H])[2H])c2ccccc2-c2cc(N(c3ccc4oc5cccc(-c6ccccc6)c5c4c3)c3cccc(-c4ccccc4)c3-c3ccc(C(C)(C)C)cc3)ccc21]

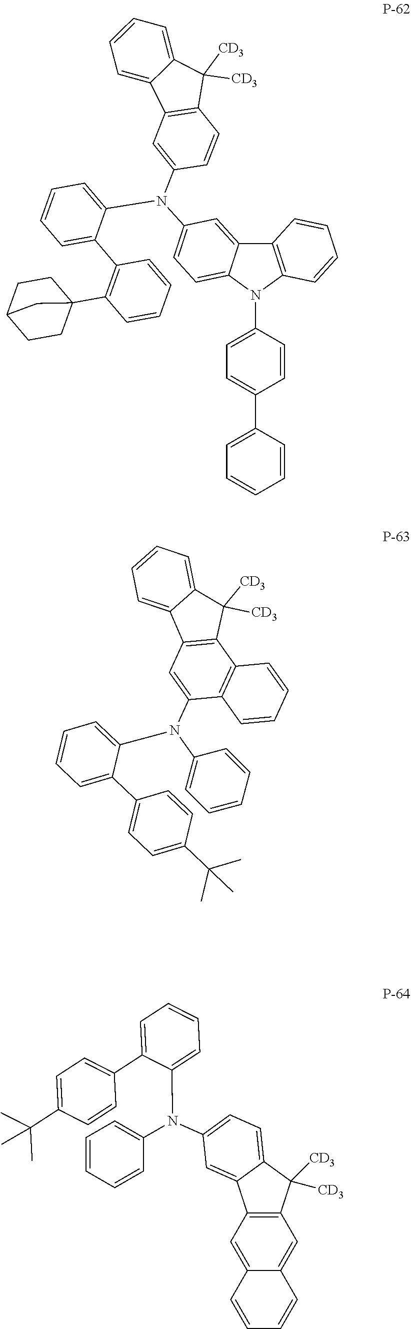 [SMILES: [2H]C([2H])([2H])C1(C([2H])([2H])[2H])c2ccccc2-c2cc(N(c3ccc4c(c3)c3ccccc3n4-c3ccc(-c4ccccc4)cc3)c3ccccc3-c3ccccc3C34CCC(CC3)C4)ccc21 ; [2H]C([2H])([2H])C1(C([2H])([2H])[2H])c2ccccc2-c2cc(N(c3ccccc3)c3ccccc3-c3ccc(C(C)(C)C)cc3)c3ccccc3c21 ; [2H]C([2H])([2H])C1(C([2H])([2H])[2H])c2ccc(N(c3ccccc3)c3ccccc3-c3ccc(C(C)(C)C)cc3)cc2-c2cc3ccccc3cc21]

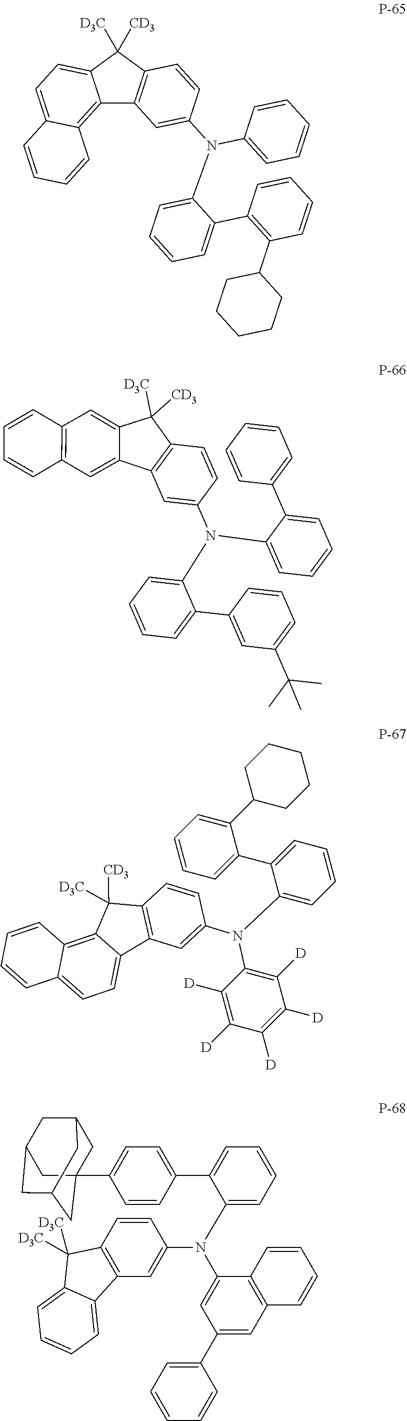

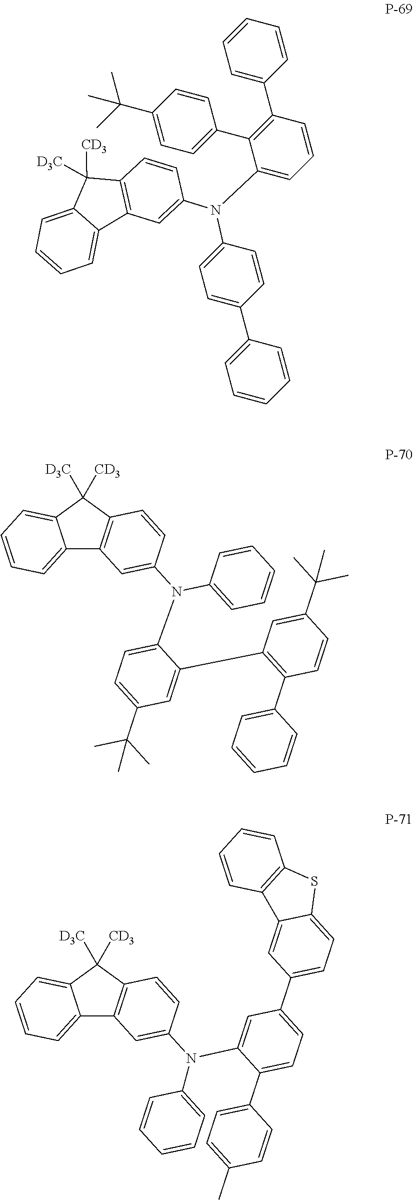 [SMILES: [2H]C([2H])([2H])C1(C([2H])([2H])[2H])c2ccccc2-c2cc(N(c3ccc(-c4ccccc4)cc3)c3cccc(-c4ccccc4)c3-c3ccc(C(C)(C)C)cc3)ccc21 ; [2H]C([2H])([2H])C1(C([2H])([2H])[2H])c2ccccc2-c2cc(N(c3ccccc3)c3ccc(C(C)(C)C)cc3-c3cc(C(C)(C)C)ccc3-c3ccccc3)ccc21 ; [2H]C([2H])([2H])C1(C([2H])([2H])[2H])c2ccccc2-c2cc(N(c3ccccc3)c3cc(-c4ccc5sc6ccccc6c5c4)ccc3-c3ccc(C)cc3)ccc21]

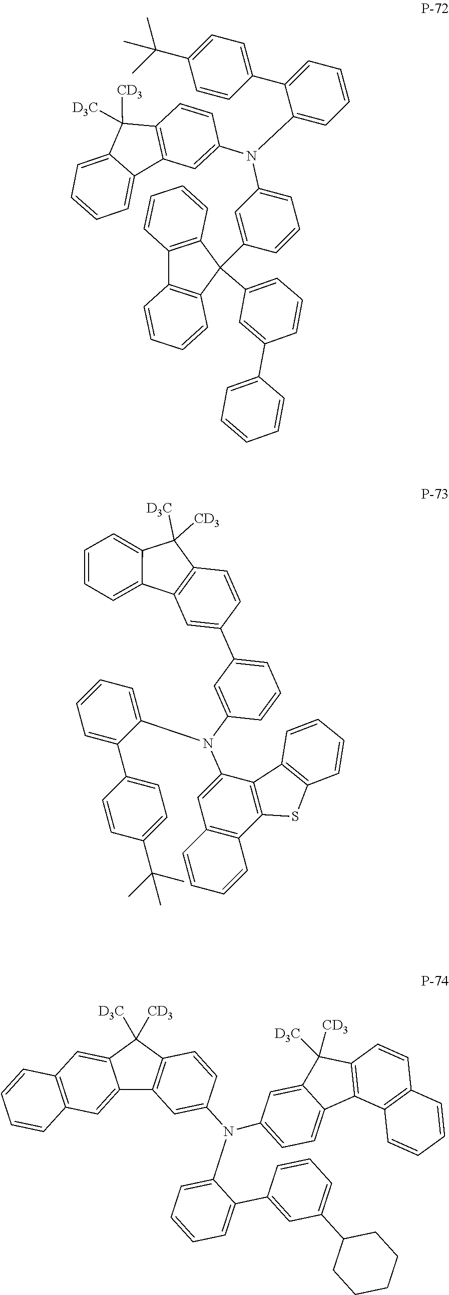 [SMILES: [2H]C([2H])([2H])C1(C([2H])([2H])[2H])c2ccccc2-c2cc(N(c3cccc(C4(c5cccc(-c6ccccc6)c5)c5ccccc5-c5ccccc54)c3)c3ccccc3-c3ccc(C(C)(C)C)cc3)ccc21 ; [2H]C([2H])([2H])C1(C([2H])([2H])[2H])c2ccccc2-c2cc(-c3cccc(N(c4ccccc4-c4ccc(C(C)(C)C)cc4)c4cc5ccccc5c5sc6ccccc6c45)c3)ccc21 ; [2H]C([2H])([2H])C1(C([2H])([2H])[2H])c2ccc(N(c3ccc4c(c3)C(C([2H])([2H])[2H])(C([2H])([2H])[2H])c3ccc5ccccc5c3-4)c3ccccc3-c3cccc(C4CCCCC4)c3)cc2-c2cc3ccccc3cc21]

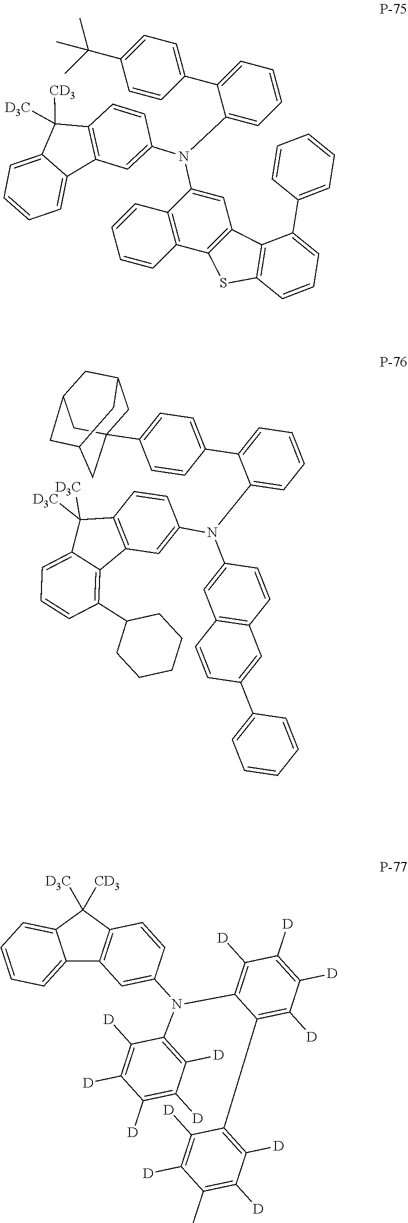 [SMILES: [2H]C([2H])([2H])C1(C([2H])([2H])[2H])c2ccccc2-c2cc(N(c3ccccc3-c3ccc(C(C)(C)C)cc3)c3cc4c(sc5cccc(-c6ccccc6)c54)c4ccccc34)ccc21 ; [2H]C([2H])([2H])C1(C([2H])([2H])[2H])c2ccc(N(c3ccc4cc(-c5ccccc5)ccc4c3)c3ccccc3-c3ccc(C45CC6CC(CC(C6)C4)C5)cc3)cc2-c2c(C3CCCCC3)cccc21 ; [2H]c1c([2H])c([2H])c(N(c2ccc3c(c2)-c2ccccc2C3(C([2H])([2H])[2H])C([2H])([2H])[2H])c2c([2H])c([2H])c([2H])c([2H])c2-c2c([2H])c([2H])c(C)c([2H])c2[2H])c([2H])c1[2H]]

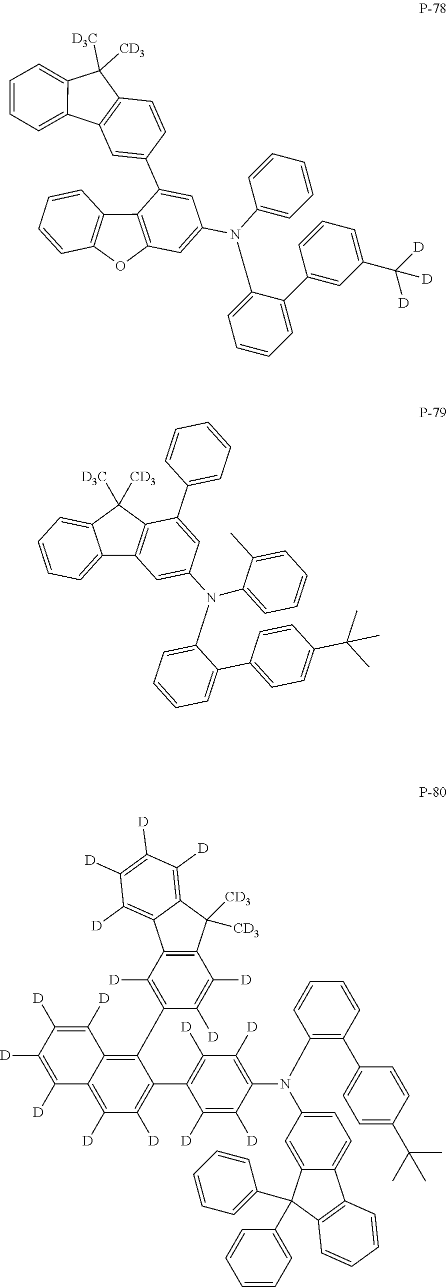 [SMILES: [2H]C([2H])([2H])c1cccc(-c2ccccc2N(c2ccccc2)c2cc(-c3ccc4c(c3)-c3ccccc3C4(C([2H])([2H])[2H])C([2H])([2H])[2H])c3c(c2)oc2ccccc23)c1 ; [2H]C([2H])([2H])C1(C([2H])([2H])[2H])c2ccccc2-c2cc(N(c3ccccc3C)c3ccccc3-c3ccc(C(C)(C)C)cc3)cc(-c3ccccc3)c21 ; [2H]c1c([2H])c([2H])c2c(c1[2H])-c1c([2H])c(-c3c(-c4c([2H])c([2H])c(N(c5ccc6c(c5)C(c5ccccc5)(c5ccccc5)c5ccccc5-6)c5ccccc5-c5ccc(C(C)(C)C)cc5)c([2H])c4[2H])c([2H])c([2H])c4c([2H])c([2H])c([2H])c([2H])c34)c([2H])c([2H])c1C2(C([2H])([2H])[2H])C([2H])([2H])[2H]]

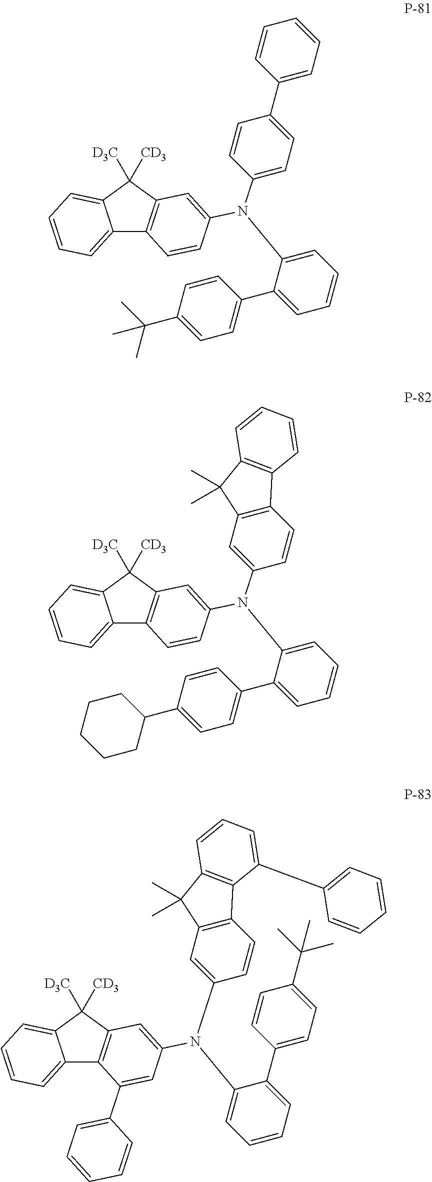 [SMILES: [2H]C([2H])([2H])C1(C([2H])([2H])[2H])c2ccccc2-c2ccc(N(c3ccc(-c4ccccc4)cc3)c3ccccc3-c3ccc(C(C)(C)C)cc3)cc21 ; [2H]C([2H])([2H])C1(C([2H])([2H])[2H])c2ccccc2-c2ccc(N(c3ccc4c(c3)C(C)(C)c3ccccc3-4)c3ccccc3-c3ccc(C4CCCCC4)cc3)cc21 ; [2H]C([2H])([2H])C1(C([2H])([2H])[2H])c2ccccc2-c2c(-c3ccccc3)cc(N(c3ccc4c(c3)C(C)(C)c3cccc(-c5ccccc5)c3-4)c3ccccc3-c3ccc(C(C)(C)C)cc3)cc21]

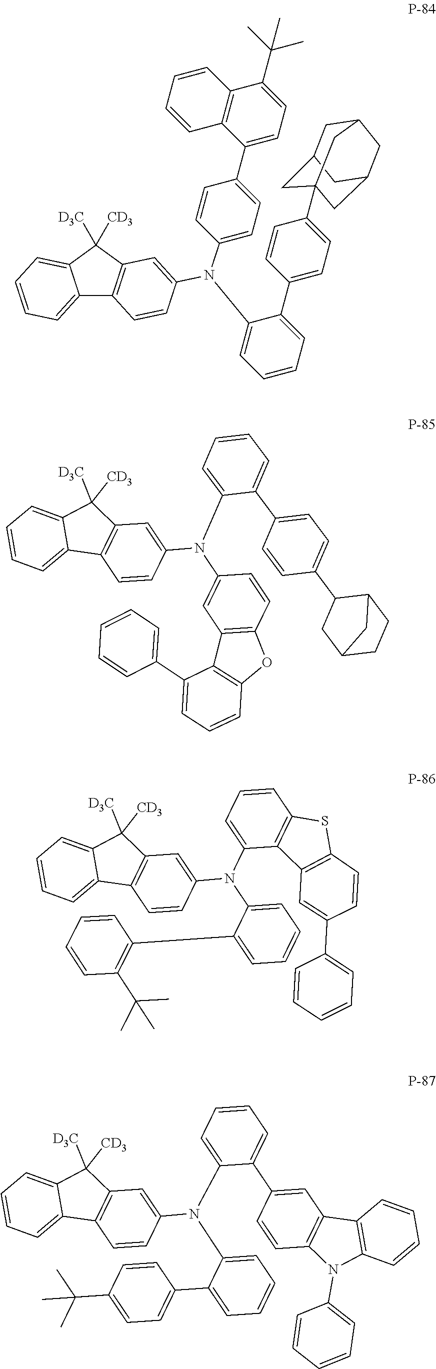

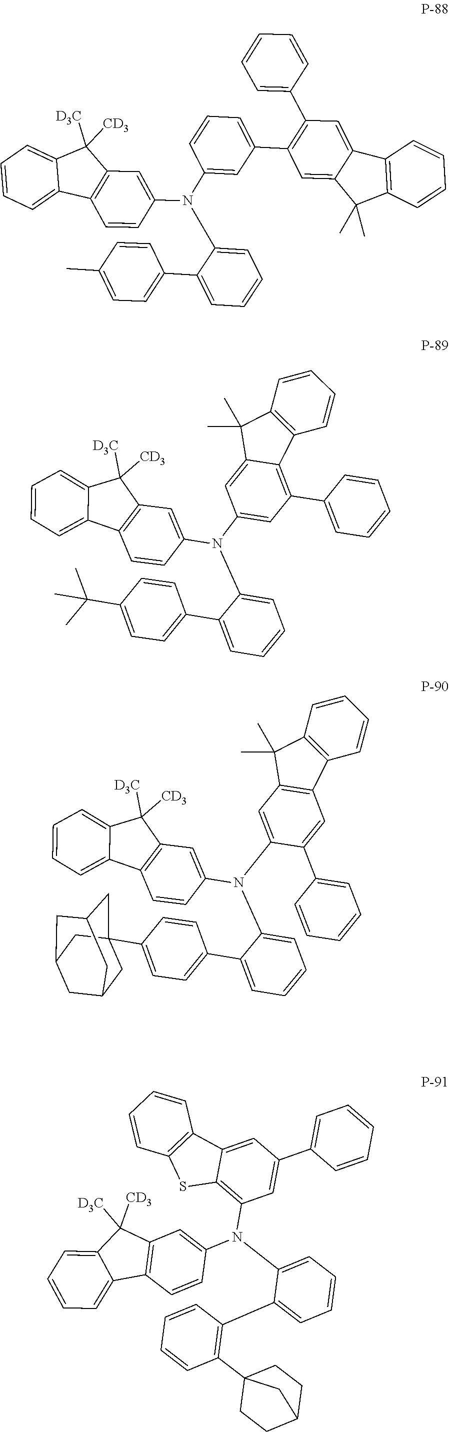

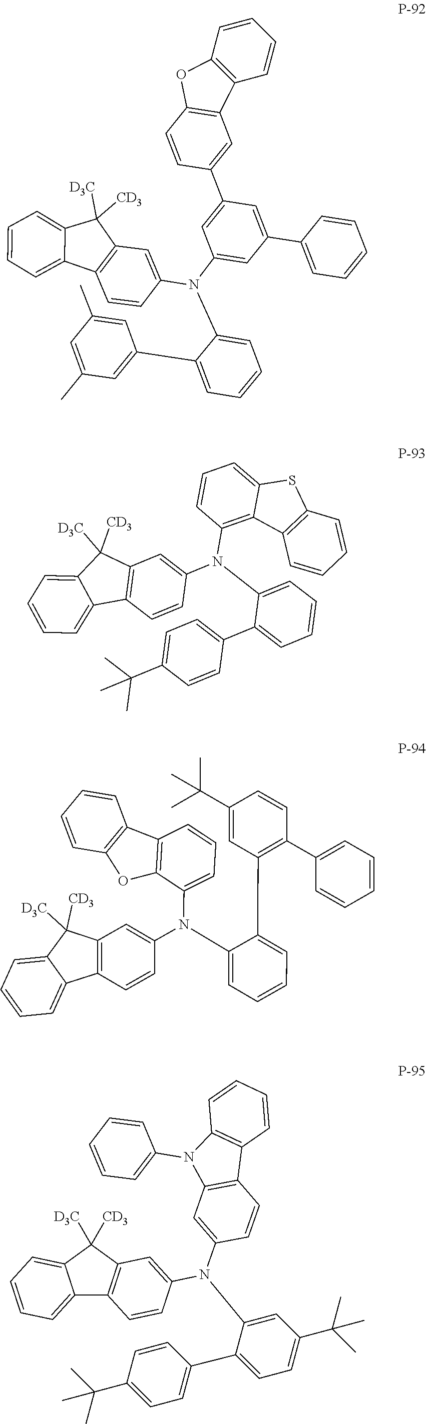 [SMILES: [2H]C([2H])([2H])C1(C([2H])([2H])[2H])c2ccccc2-c2ccc(N(c3cc(-c4ccccc4)cc(-c4ccc5oc6ccccc6c5c4)c3)c3ccccc3-c3cc(C)cc(C)c3)cc21 ; [2H]C([2H])([2H])C1(C([2H])([2H])[2H])c2ccccc2-c2ccc(N(c3ccccc3-c3ccc(C(C)(C)C)cc3)c3cccc4sc5ccccc5c34)cc21 ; [2H]C([2H])([2H])C1(C([2H])([2H])[2H])c2ccccc2-c2ccc(N(c3ccccc3-c3cc(C(C)(C)C)ccc3-c3ccccc3)c3cccc4c3oc3ccccc34)cc21 ; [2H]C([2H])([2H])C1(C([2H])([2H])[2H])c2ccccc2-c2ccc(N(c3ccc4c5ccccc5n(-c5ccccc5)c4c3)c3cc(C(C)(C)C)ccc3-c3ccc(C(C)(C)C)cc3)cc21]

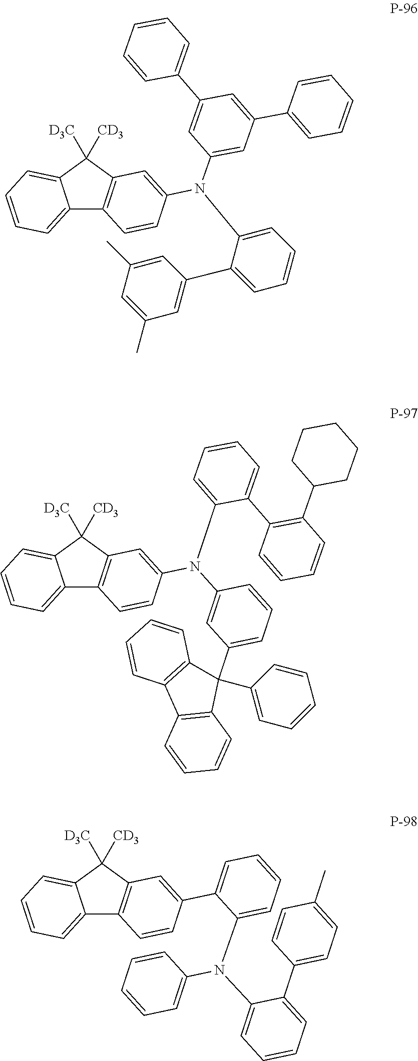 [SMILES: [2H]C([2H])([2H])C1(C([2H])([2H])[2H])c2ccccc2-c2ccc(N(c3cc(-c4ccccc4)cc(-c4ccccc4)c3)c3ccccc3-c3cc(C)cc(C)c3)cc21 ; [2H]C([2H])([2H])C1(C([2H])([2H])[2H])c2ccccc2-c2ccc(N(c3cccc(C4(c5ccccc5)c5ccccc5-c5ccccc54)c3)c3ccccc3-c3ccccc3C3CCCCC3)cc21 ; [2H]C([2H])([2H])C1(C([2H])([2H])[2H])c2ccccc2-c2ccc(-c3ccccc3N(c3ccccc3)c3ccccc3-c3ccc(C)cc3)cc21]

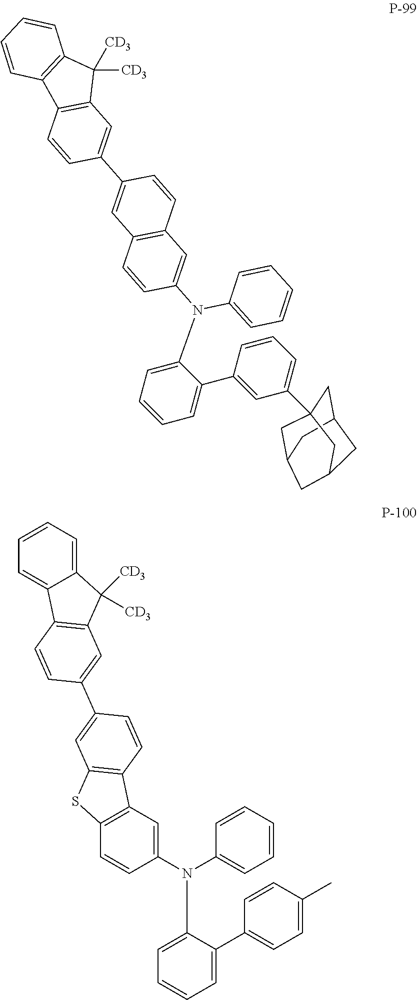 [SMILES: [2H]C([2H])([2H])C1(C([2H])([2H])[2H])c2ccccc2-c2ccc(-c3ccc4cc(N(c5ccccc5)c5ccccc5-c5cccc(C67CC8CC(CC(C8)C6)C7)c5)ccc4c3)cc21 ; [2H]C([2H])([2H])C1(C([2H])([2H])[2H])c2ccccc2-c2ccc(-c3ccc4c(c3)sc3ccc(N(c5ccccc5)c5ccccc5-c5ccc(C)cc5)cc34)cc21]

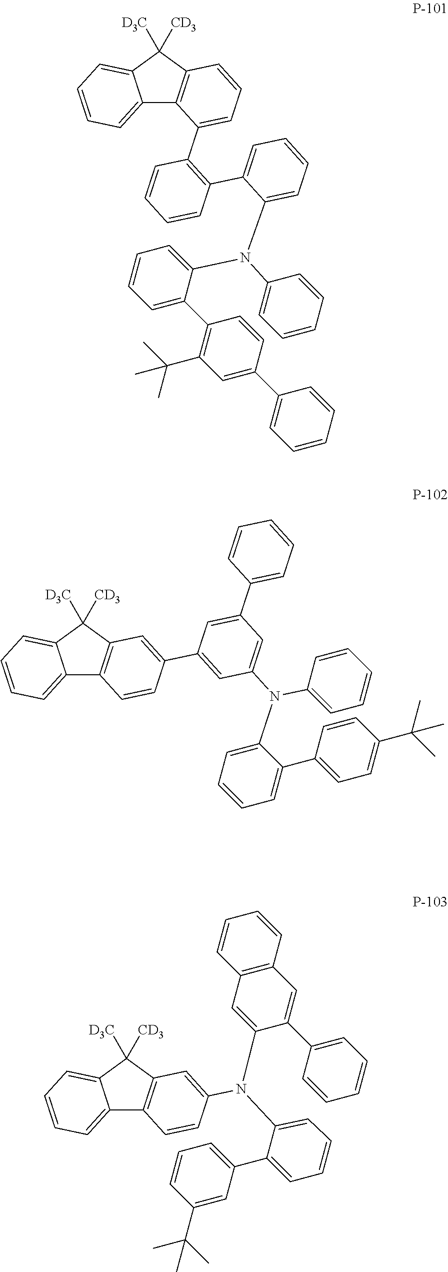 [SMILES: [2H]C([2H])([2H])C1(C([2H])([2H])[2H])c2ccccc2-c2c(-c3ccccc3-c3ccccc3N(c3ccccc3)c3ccccc3-c3ccc(-c4ccccc4)cc3C(C)(C)C)cccc21 ; [2H]C([2H])([2H])C1(C([2H])([2H])[2H])c2ccccc2-c2ccc(-c3cc(-c4ccccc4)cc(N(c4ccccc4)c4ccccc4-c4ccc(C(C)(C)C)cc4)c3)cc21 ; [2H]C([2H])([2H])C1(C([2H])([2H])[2H])c2ccccc2-c2ccc(N(c3ccccc3-c3cccc(C(C)(C)C)c3)c3cc4ccccc4cc3-c3ccccc3)cc21]

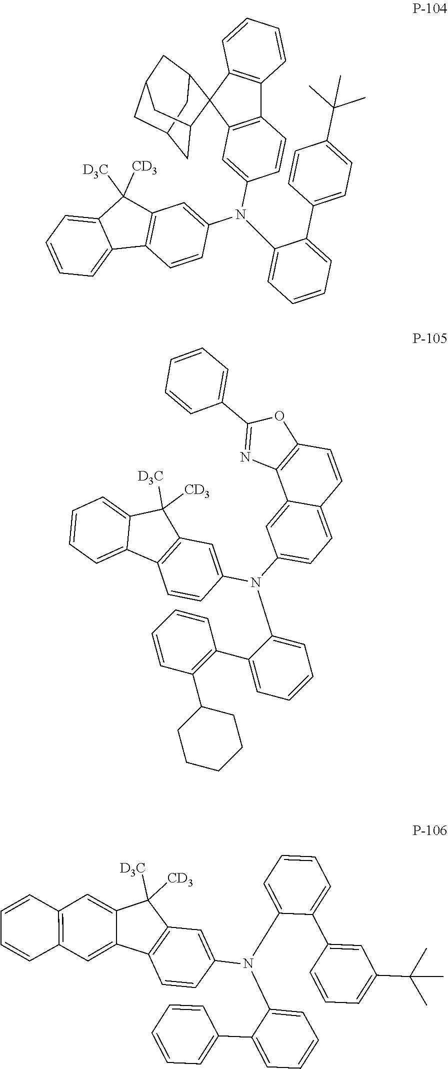 [SMILES: [2H]C([2H])([2H])C1(C([2H])([2H])[2H])c2ccccc2-c2ccc(N(c3ccc4c(c3)C3(c5ccccc5-4)C4CC5CC(C4)CC3C5)c3ccccc3-c3ccc(C(C)(C)C)cc3)cc21 ; [2H]C([2H])([2H])C1(C([2H])([2H])[2H])c2ccccc2-c2ccc(N(c3ccc4ccc5oc(-c6ccccc6)nc5c4c3)c3ccccc3-c3ccccc3C3CCCCC3)cc21 ; [2H]C([2H])([2H])C1(C([2H])([2H])[2H])c2cc(N(c3ccccc3-c3ccccc3)c3ccccc3-c3cccc(C(C)(C)C)c3)ccc2-c2cc3ccccc3cc21]

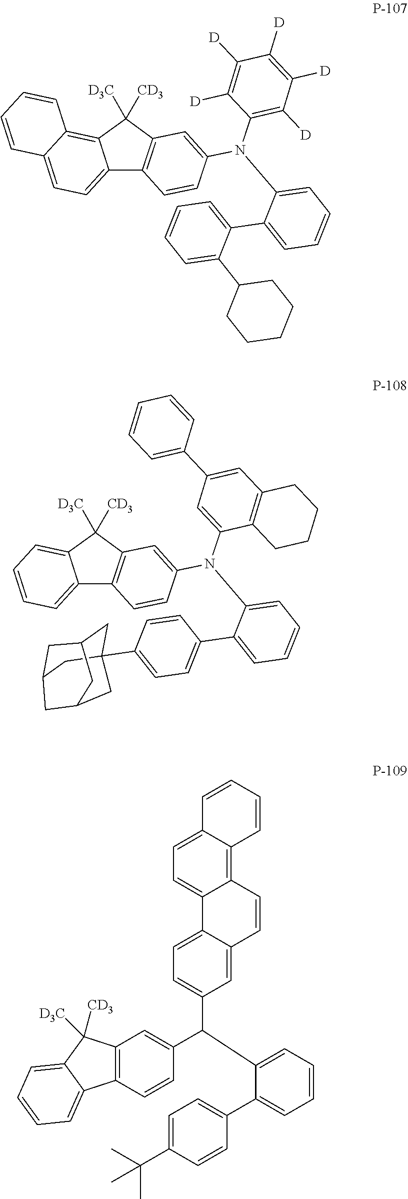 [SMILES: [2H]c1c([2H])c([2H])c(N(c2ccc3c(c2)C(C([2H])([2H])[2H])(C([2H])([2H])[2H])c2c-3ccc3ccccc23)c2ccccc2-c2ccccc2C2CCCCC2)c([2H])c1[2H] ; [2H]C([2H])([2H])C1(C([2H])([2H])[2H])c2ccccc2-c2ccc(N(c3ccccc3-c3ccc(C45CC6CC(CC(C6)C4)C5)cc3)c3cc(-c4ccccc4)cc4c3CCCC4)cc21 ; [2H]C([2H])([2H])C1(C([2H])([2H])[2H])c2ccccc2-c2ccc(C(c3ccc4c(ccc5c6ccccc6ccc45)c3)c3ccccc3-c3ccc(C(C)(C)C)cc3)cc21]

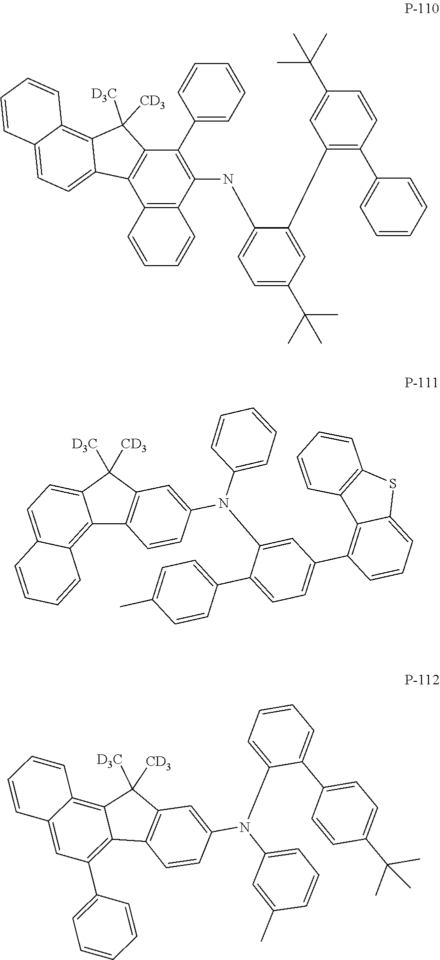 [SMILES: [2H]C([2H])([2H])C1(C([2H])([2H])[2H])c2c(-c3ccccc3)c(Nc3ccc(C(C)(C)C)cc3-c3cc(C(C)(C)C)ccc3-c3ccccc3)c3ccccc3c2-c2ccc3ccccc3c21 ; [2H]C([2H])([2H])C1(C([2H])([2H])[2H])c2cc(N(c3ccccc3)c3cc(-c4cccc5sc6ccccc6c45)ccc3-c3ccc(C)cc3)ccc2-c2c1ccc1ccccc21 ; [2H]C([2H])([2H])C1(C([2H])([2H])[2H])c2cc(N(c3cccc(C)c3)c3ccccc3-c3ccc(C(C)(C)C)cc3)ccc2-c2c(-c3ccccc3)cc3ccccc3c21]

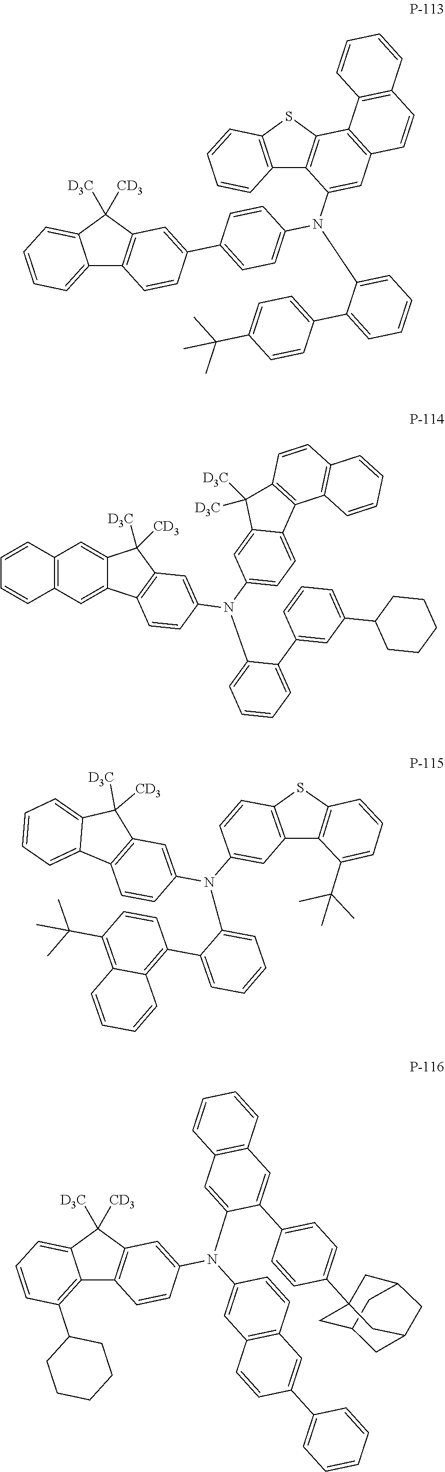

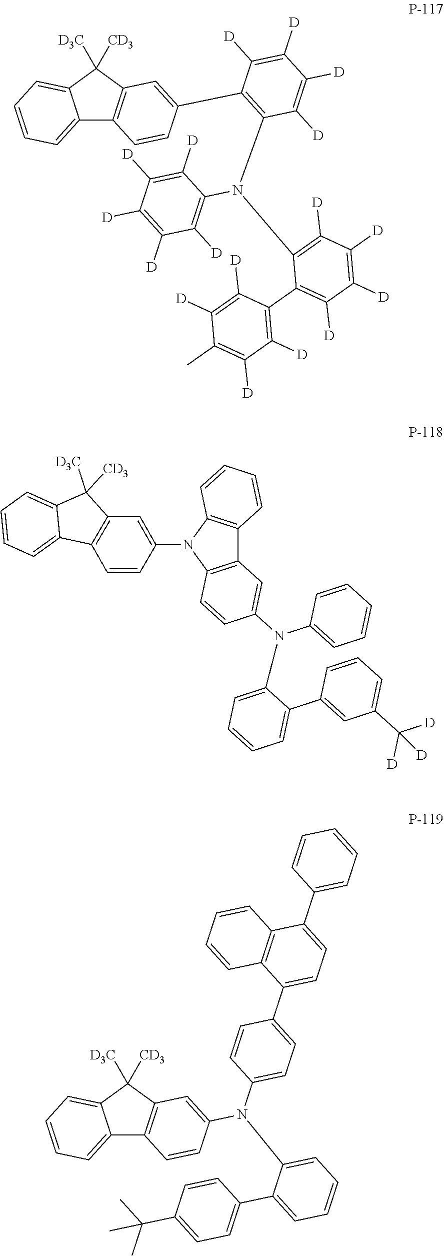 [SMILES: [2H]c1c([2H])c([2H])c(N(c2c([2H])c([2H])c([2H])c([2H])c2-c2ccc3c(c2)C(C([2H])([2H])[2H])(C([2H])([2H])[2H])c2ccccc2-3)c2c([2H])c([2H])c([2H])c([2H])c2-c2c([2H])c([2H])c(C)c([2H])c2[2H])c([2H])c1[2H] ; [2H]C([2H])([2H])c1cccc(-c2ccccc2N(c2ccccc2)c2ccc3c(c2)c2ccccc2n3-c2ccc3c(c2)C(C([2H])([2H])[2H])(C([2H])([2H])[2H])c2ccccc2-3)c1 ; [2H]C([2H])([2H])C1(C([2H])([2H])[2H])c2ccccc2-c2ccc(N(c3ccc(-c4ccc(-c5ccccc5)c5ccccc45)cc3)c3ccccc3-c3ccc(C(C)(C)C)cc3)cc21]

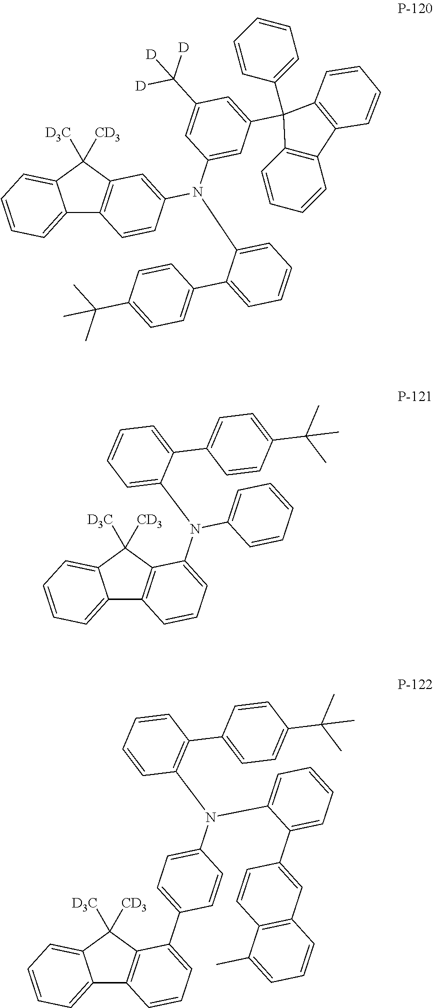 [SMILES: [2H]C([2H])([2H])c1cc(N(c2ccc3c(c2)C(C([2H])([2H])[2H])(C([2H])([2H])[2H])c2ccccc2-3)c2ccccc2-c2ccc(C(C)(C)C)cc2)cc(C2(c3ccccc3)c3ccccc3-c3ccccc32)c1 ; [2H]C([2H])([2H])C1(C([2H])([2H])[2H])c2ccccc2-c2cccc(N(c3ccccc3)c3ccccc3-c3ccc(C(C)(C)C)cc3)c21 ; [2H]C([2H])([2H])C1(C([2H])([2H])[2H])c2ccccc2-c2cccc(-c3ccc(N(c4ccccc4-c4ccc(C(C)(C)C)cc4)c4ccccc4-c4ccc5c(C)cccc5c4)cc3)c21]

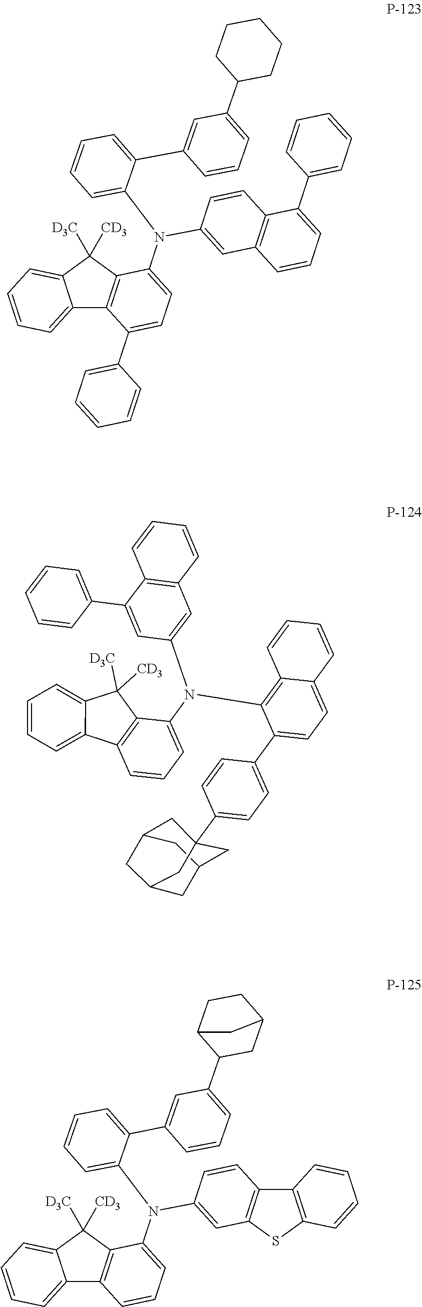 [SMILES: [2H]C([2H])([2H])C1(C([2H])([2H])[2H])c2ccccc2-c2c(-c3ccccc3)ccc(N(c3ccc4c(-c5ccccc5)cccc4c3)c3ccccc3-c3cccc(C4CCCCC4)c3)c21 ; [2H]C([2H])([2H])C1(C([2H])([2H])[2H])c2ccccc2-c2cccc(N(c3cc(-c4ccccc4)c4ccccc4c3)c3c(-c4ccc(C56CC7CC(CC(C7)C5)C6)cc4)ccc4ccccc34)c21 ; [2H]C([2H])([2H])C1(C([2H])([2H])[2H])c2ccccc2-c2cccc(N(c3ccc4c(c3)sc3ccccc34)c3ccccc3-c3cccc(C4CC5CCC4C5)c3)c21]

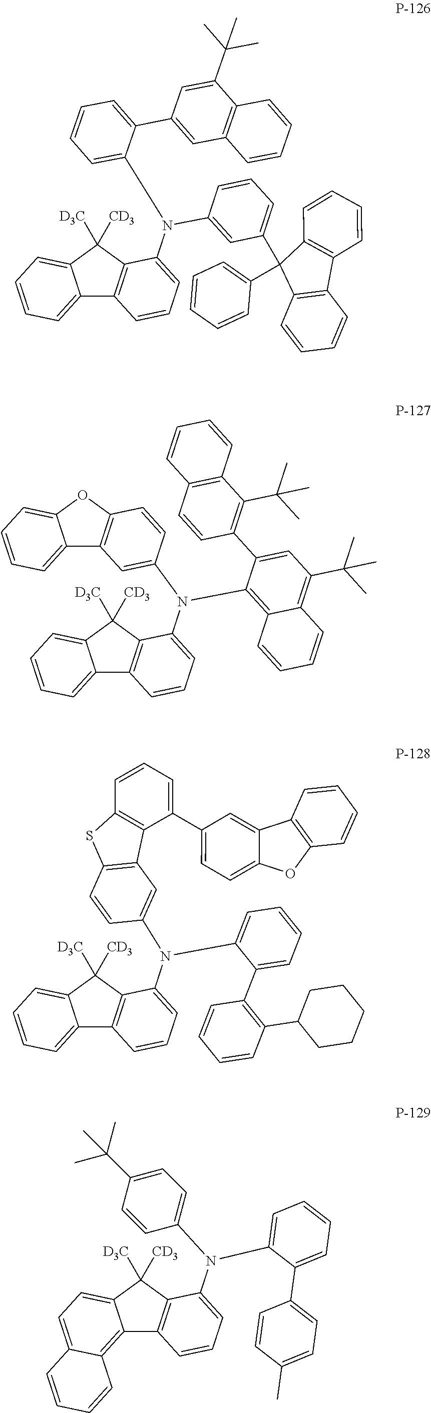

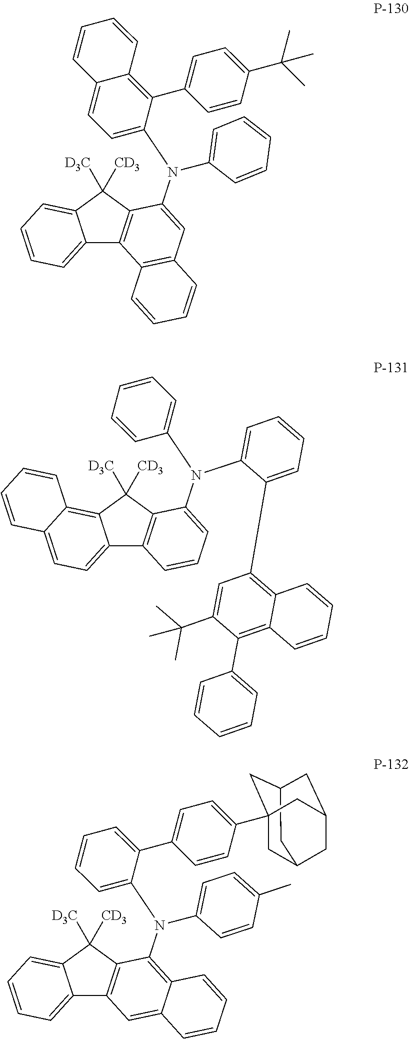 [SMILES: [2H]C([2H])([2H])C1(C([2H])([2H])[2H])c2ccccc2-c2c1c(N(c1ccccc1)c1ccc3ccccc3c1-c1ccc(C(C)(C)C)cc1)cc1ccccc21 ; [2H]C([2H])([2H])C1(C([2H])([2H])[2H])c2c(cccc2N(c2ccccc2)c2ccccc2-c2cc(C(C)(C)C)c(-c3ccccc3)c3ccccc23)-c2ccc3ccccc3c21 ; [2H]C([2H])([2H])C1(C([2H])([2H])[2H])c2ccccc2-c2cc3ccccc3c(N(c3ccc(C)cc3)c3ccccc3-c3ccc(C45CC6CC(CC(C6)C4)C5)cc3)c21]

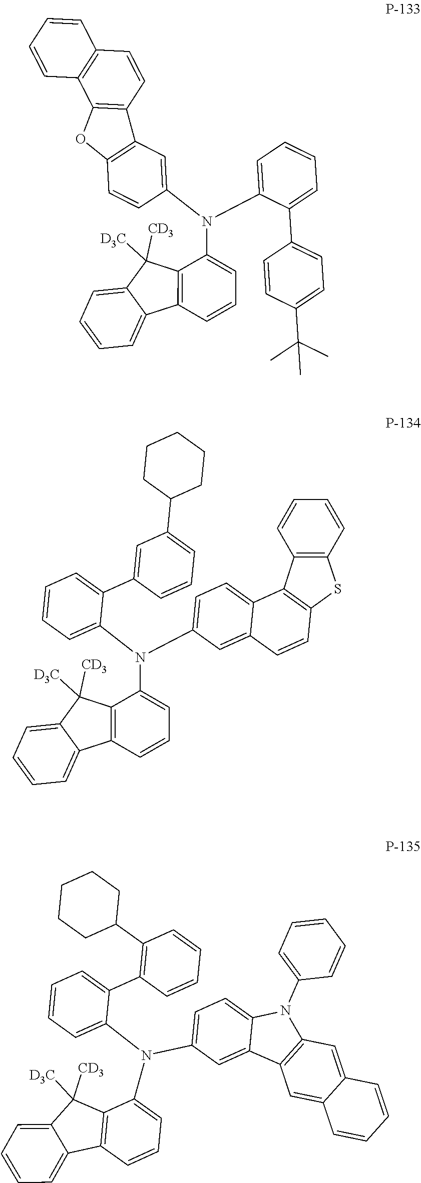 [SMILES: [2H]C([2H])([2H])C1(C([2H])([2H])[2H])c2ccccc2-c2cccc(N(c3ccc4oc5c6ccccc6ccc5c4c3)c3ccccc3-c3ccc(C(C)(C)C)cc3)c21 ; [2H]C([2H])([2H])C1(C([2H])([2H])[2H])c2ccccc2-c2cccc(N(c3ccc4c(ccc5sc6ccccc6c54)c3)c3ccccc3-c3cccc(C4CCCCC4)c3)c21 ; [2H]C([2H])([2H])C1(C([2H])([2H])[2H])c2ccccc2-c2cccc(N(c3ccc4c(c3)c3cc5ccccc5cc3n4-c3ccccc3)c3ccccc3-c3ccccc3C3CCCCC3)c21]

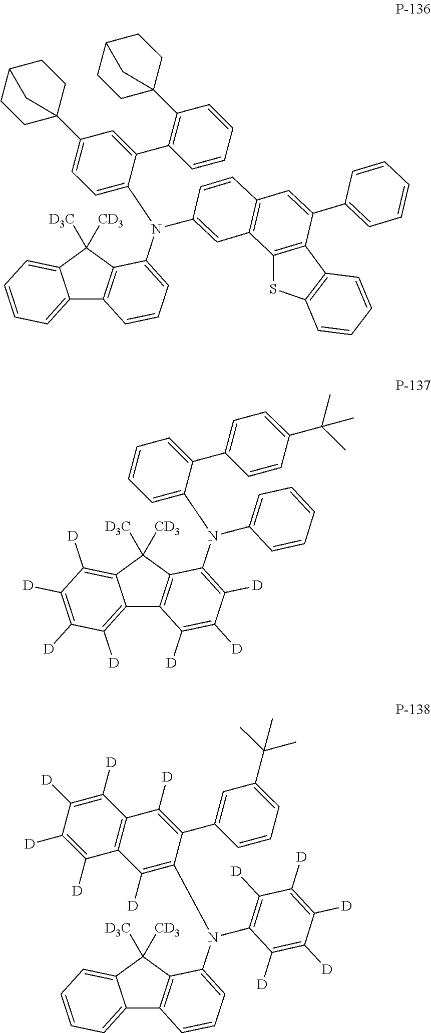 [SMILES: [2H]C([2H])([2H])C1(C([2H])([2H])[2H])c2ccccc2-c2cccc(N(c3ccc4cc(-c5ccccc5)c5c6ccccc6sc5c4c3)c3ccc(C45CCC(CC4)C5)cc3-c3ccccc3C34CCC(CC3)C4)c21 ; [2H]c1c([2H])c([2H])c2c(c1[2H])-c1c([2H])c([2H])c([2H])c(N(c3ccccc3)c3ccccc3-c3ccc(C(C)(C)C)cc3)c1C2(C([2H])([2H])[2H])C([2H])([2H])[2H] ; [2H]c1c([2H])c([2H])c(N(c2cccc3c2C(C([2H])([2H])[2H])(C([2H])([2H])[2H])c2ccccc2-3)c2c(-c3cccc(C(C)(C)C)c3)c([2H])c3c([2H])c([2H])c([2H])c([2H])c3c2[2H])c([2H])c1[2H]]

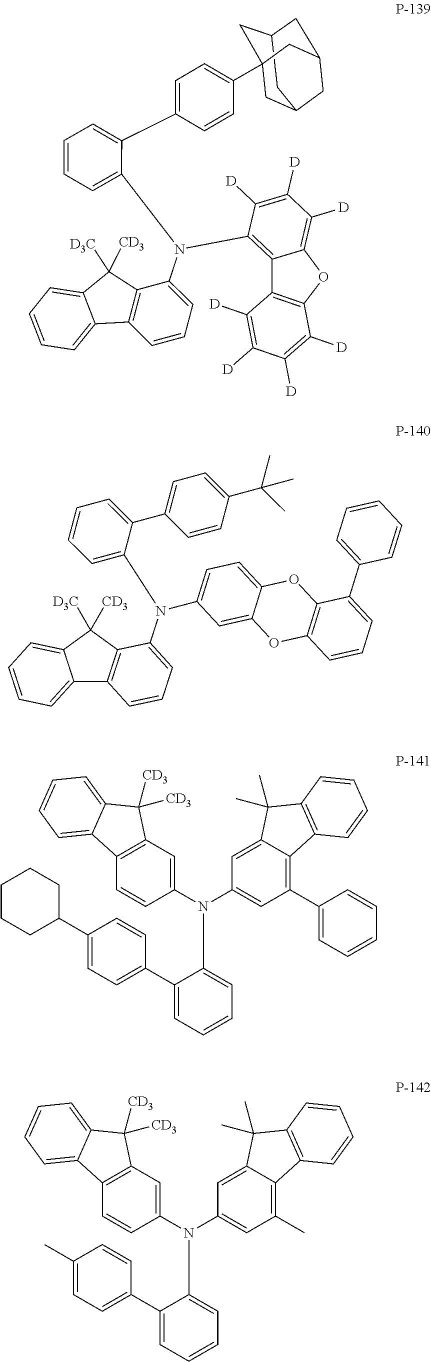

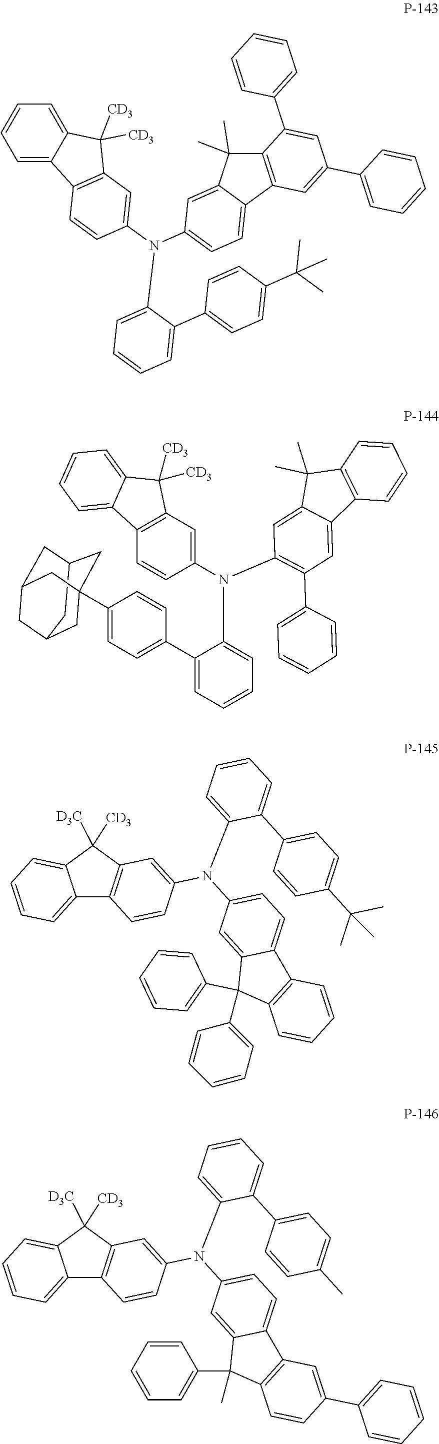

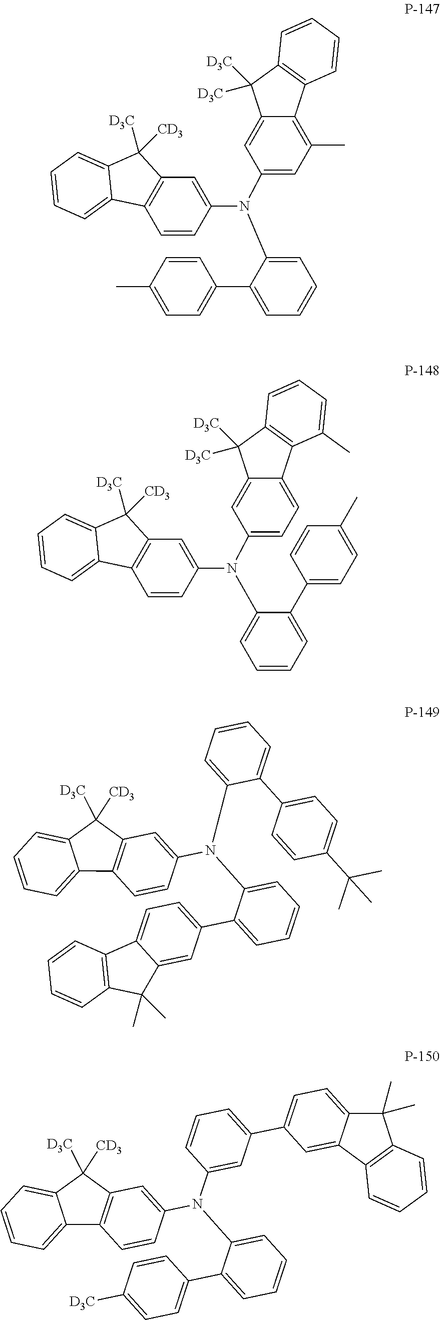

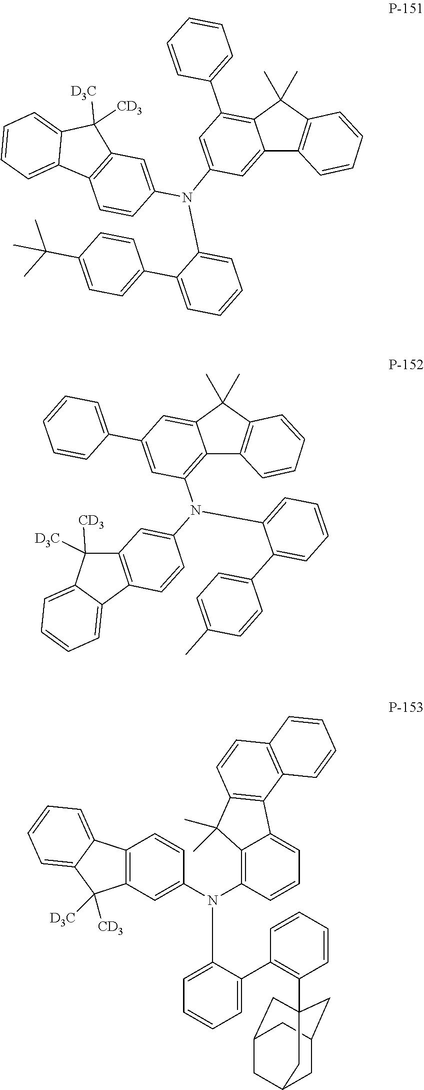 [SMILES: [2H]C([2H])([2H])C1(C([2H])([2H])[2H])c2ccccc2-c2ccc(N(c3cc(-c4ccccc4)c4c(c3)-c3ccccc3C4(C)C)c3ccccc3-c3ccc(C(C)(C)C)cc3)cc21 ; [2H]C([2H])([2H])C1(C([2H])([2H])[2H])c2ccccc2-c2ccc(N(c3ccccc3-c3ccc(C)cc3)c3cc(-c4ccccc4)cc4c3-c3ccccc3C4(C)C)cc21 ; [2H]C([2H])([2H])C1(C([2H])([2H])[2H])c2ccccc2-c2ccc(N(c3ccccc3-c3ccccc3C34CC5CC(CC(C5)C3)C4)c3cccc4c3C(C)(C)c3ccc5ccccc5c3-4)cc21]

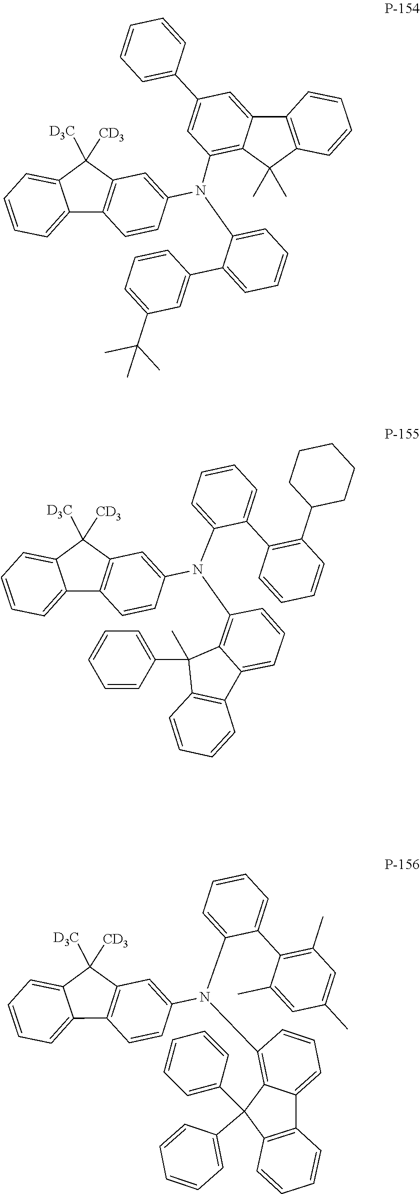 [SMILES: [2H]C([2H])([2H])C1(C([2H])([2H])[2H])c2ccccc2-c2ccc(N(c3ccccc3-c3cccc(C(C)(C)C)c3)c3cc(-c4ccccc4)cc4c3C(C)(C)c3ccccc3-4)cc21 ; [2H]C([2H])([2H])C1(C([2H])([2H])[2H])c2ccccc2-c2ccc(N(c3ccccc3-c3ccccc3C3CCCCC3)c3cccc4c3C(C)(c3ccccc3)c3ccccc3-4)cc21 ; [2H]C([2H])([2H])C1(C([2H])([2H])[2H])c2ccccc2-c2ccc(N(c3ccccc3-c3c(C)cc(C)cc3C)c3cccc4c3C(c3ccccc3)(c3ccccc3)c3ccccc3-4)cc21]

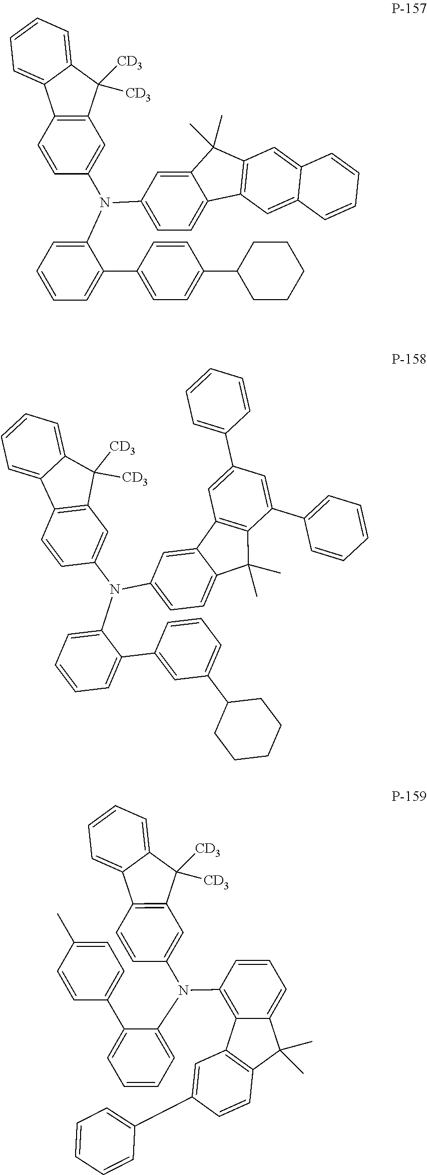

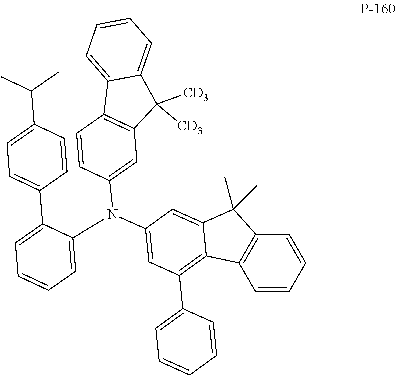 [SMILES: [2H]C([2H])([2H])C1(C([2H])([2H])[2H])c2ccccc2-c2ccc(N(c3cc(-c4ccccc4)c4c(c3)C(C)(C)c3ccccc3-4)c3ccccc3-c3ccc(C(C)C)cc3)cc21]

In another aspect, the present invention provides a method for reusing a compound of Formula 1 comprising:
recovering a crude organic light emitting material comprising the compound of Formula 1 from a deposition apparatus used in the process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.

The step of removing impurities from the crude organic light emitting material recovered from the deposition apparatus may preferably comprise performing a pre-purification process to obtain a purity of 98% or more by recrystallization in a recrystallization solvent.

The recrystallization solvent may be preferably a polar solvent having a polarity index (PI) of 5.5 to 7.2.

The recrystallization solvent may preferably be used by mixing a polar solvent having a polarity value of 5.5 to 7.2 and a non-polar solvent having a polarity value of 2.0 to 4.7.

When a mixture of a polar solvent and a non-polar solvent is used, the recrystallization solvent may be used in an amount of 15% (v/v) or less of the non-polar solvent compared to the polar solvent.

The recrystallization solvent is preferably a single solvent of N-Methylpyrrolidone (NMP); or a polar solvent mixed any one selected from the group consisting of 1,3-Dimethyl-2-imidazolidinone, 2-pyrrolidone, N,N-Dimethyl formamide, Dimethyl acetamide, and Dimethyl sulfoxide to the N-Methylpyrrolidone; or alone; or mixed non-polar solvents; selected from the group consisting of Toluene, Dichloromethane (DCM), Dichloroethane (DCE), Tetrahydrofuran (THF), Chloroform, Ethyl acetate and Butanone; or a mixture of a polar solvent and a non-polar solvent.

The pre-purification process may comprise a step of precipitating crystals of by cooling to 0° C. to 5° C. after dissolving the crude organic light emitting material recovered from the deposition apparatus in a polar solvent at 90° C. to 120° C.

The pre-purification process may comprise a step of precipitating crystals by cooling to 35° C. to 40° C., adding a non-polar solvent, and then cooling to 0° C. to 5° C. after dissolving the crude organic light emitting material recovered from the deposition apparatus in a polar solvent at 90° C. to 120° C.

The pre-purification process may comprise a step of precipitating crystals while concentrating the solvent and removing the non-polar solvent, after dissolving the crude organic light emitting material recovered from the deposition apparatus in a non-polar solvent.

The pre-purification process may comprise a step of recrystallizing again with a non-polar solvent after recrystallizing first with a polar solvent.

The step of purifying the recovered impurities to a purity of 99.9% or higher may comprise performing an adsorption separation process to adsorb and remove impurities by adsorbing on the adsorbent.

The adsorbent may be activated carbon, silica gel, alumina, or a material for known adsorption purposes.

The step of purifying the recovered impurities to a purity of 99.9% or higher may comprise performing sublimation purification.

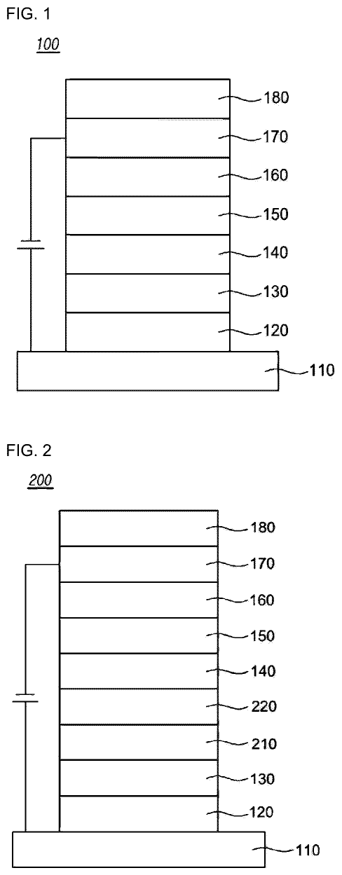

Referring to FIG. 1, the organic electronic element (100) according to the present invention comprises a first electrode (110), a second electrode (170), an organic material layer comprising single compound or 2 or more compounds represented by Formula 1 between the first electrode (110) and the second electrode (170). Wherein, the first electrode (110) may be an anode or a positive electrode, and the second electrode (170) may be a cathode or a negative electrode. In the case of an inverted organic electronic element, the first electrode may be a cathode, and the second electrode may be an anode.

The organic material layer may sequentially comprise a hole injection layer (120), a hole transport layer (130), an emitting layer (140), an electron transport layer (150), and an electron injection layer (160) on the first electrode (110). Here, the remaining layers except the emitting layer (140) may not be formed. The organic material layer may further comprise a hole blocking layer, an electron blocking layer, an emitting-auxiliary layer (220), a buffer layer (210), etc., and the electron transport layer (150), etc. may serve as a hole blocking layer (see FIG. 2).

Also, the organic electronic element according to an embodiment of the present invention may further include a protective layer or a light efficiency enhancing layer (180). The light efficiency enhancing layer may be formed on a surface of both sides of the first electrode that is not in contact with the organic material layer or on a surface of both sides of the second electrode that is not in contact with the organic material layer. The compound or materials for organic electronic element according to an embodiment of the present invention applied to the organic material layer may be used as a material for a hole injection layer (120), a hole transport layer (130), an emitting-auxiliary layer (220), an electron transport auxiliary layer, an electron transport layer (150), an electron injection layer (160), a host or dopant of an emitting layer (140), or the light efficiency enhancing layer. Preferably, for example, a composition for an organic electronic element comprising a compound according to Formula 1 of the present invention can be used as a host material of an emitting auxiliary layer.

The organic material layer may comprise 2 or more stacks comprising a hole transport layer, an emitting layer and an electron transport layer sequentially formed on the anode, and may further comprise a charge generation layer formed between the 2 or more stacks (see FIG. 3).

Otherwise, even if the same core is used, the band gap, the electrical characteristics, the interface characteristics, etc. may vary depending on which substituent is bonded at which position, therefore the choice of core and the combination of sub-substituents associated therewith is also very important, and in particular, when the optimal combination of energy levels and T1 values, and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (120), the hole transport layer (130), the emitting layer (140), the electron transport layer (150), and the electron injection layer (160) is formed thereon, and then depositing a material usable as a cathode thereon can manufacture an organic electroluminescent device according to an embodiment of the present invention.

Also, the present invention provides the organic electronic element wherein the organic material layer is formed by one of a spin coating process, a nozzle printing process, an inkjet printing process, a slot coating process, a dip coating process or a roll-to-roll process, and the organic material layer provides an organic electronic element comprising the compound or a composition for an organic electronic element as an electron transport material.

As another specific example, the present invention provides an organic electronic element used by mixing the same or different compounds of the compound represented by Formula 1 to the organic material layer.

Also, the present invention provides an emitting-auxiliary layer composition comprising a compound represented by Formula 1, and provides an organic electronic element comprising the emitting-auxiliary layer.

Also, the present invention also provides an electronic device comprising a display device comprising the organic electronic element; and a control unit for driving the display device.

According to another aspect, the present invention provides a display device wherein the organic electronic element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis examples of the compound represented by Formula 1 according to the present invention and preparation examples of the organic electronic element will be described in detail by way of example, but are not limited to the following examples.

### [Synthesis Example 1] A Compound Represented by Formula 1

The compound (final products) represented by Formula 1 according to the present invention is synthesized as in the Reaction Scheme 1, but is not limited thereto.

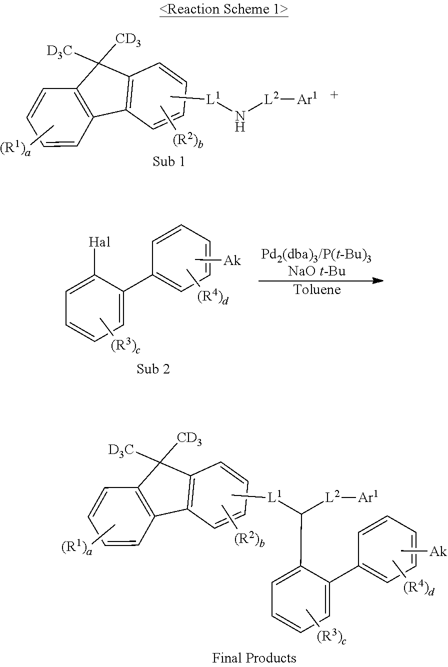

Wherein,
R¹, R², R³, R⁴, a, b, c, d, Ar¹, L¹, L² and Ak are the same as defined in Formula 1,
Hal is one of Cl, Br, and I.

### I. Synthesis of Final Product

### 1. Synthesis of P-1

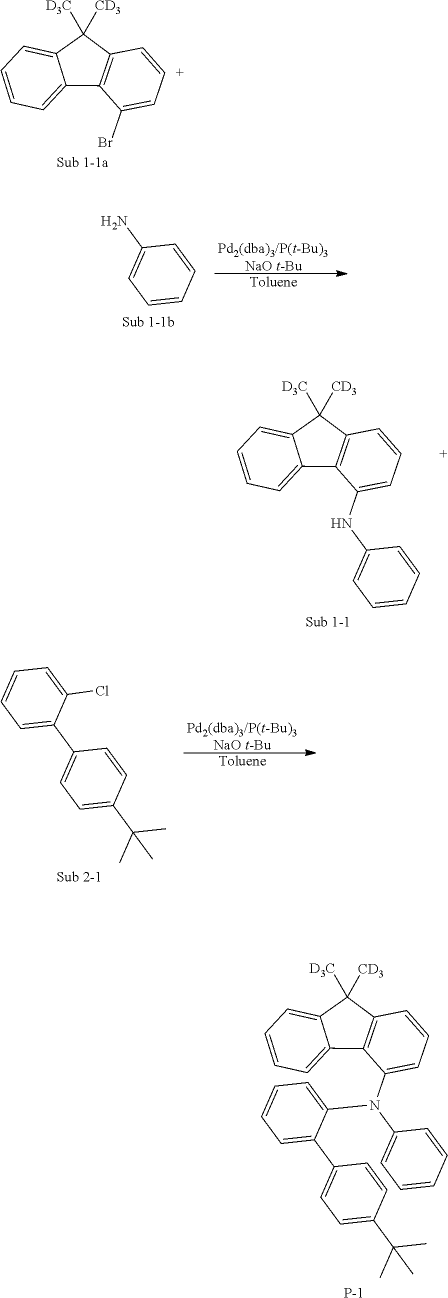

### 1) Synthesis of Sub 1-1

Sub 1-1a (100.0 g, 358.2 mmol) was dissolved in toluene (1791 mL) in a round-bottom flask, and then Sub 1-1b (33.4 g, 358.2 mmol), Pd₂(dba)₃ (9.8 g, 10.7 mmol), P(t-Bu)₃ (4.4 g, 21.5 mmol), and NaOt-Bu (68.8 g, 716.3 mmol) were added and stirred at 120° C. After the reaction was completed, the organic layer was extracted with CH₂Cl₂ and water, dried with MgSO₄, concentrated, and the resulting compound was recrystallized using a silica gel column to obtain 77.9 g of the product. (Yield: 74.6%)

### 2) Synthesis of P-1

Sub 1-1 (50.0 g, 171.6 mmol), Sub 2-1 (42.0 g, 171.6 mmol), Pd₂(dba)₃ (4.7 g, 5.2 mmol), P(t-Bu)₃ (2.1 g, 10.3 mmol), NaOt-Bu (33.0 g, 343.1 mmol), and toluene (858 mL) were added to a round-bottomed flask in the same manner as Sub 1-1 to obtain 62.2 g of the product. (Yield: 72.5%)

### 2. Synthesis of P-17

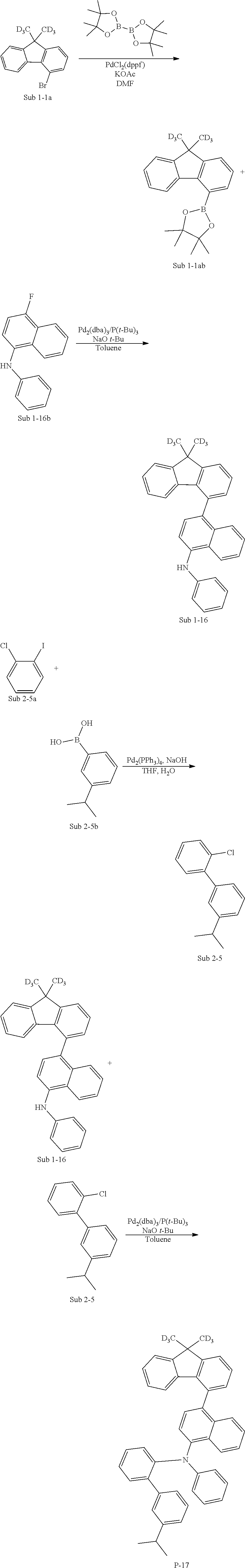

### 1) Synthesis of Sub 1-1ab

Sub 1-1a (100.0 g, 358.2 mmol) was placed in a round-bottom flask and dissolved in DMF (1791 ml). 4,4,4′,4′,5,5,5′,5′-octamethyl-2,2′-bi(1,3,2-dioxaborolane) (118.2 g, 465.6 mmol), Pd(dppf)Cl₂ (13.1 g, 17.9 mmol), KOAc (105.5 g, 1074.5 mmol) were added, and the mixture was stirred at 150° C. for 2 hours. After the reaction was completed, the organic layer was extracted with CH₂Cl₂ and water, dried with MgSO₄, concentrated, and the resulting compound was recrystallized using a silica gel column to obtain 95.4 g of the product. (Yield: 81.6%)

### 2) Synthesis of P-16

Sub 1-1ab (80.0 g, 245.2 mmol), Sub 1-16b (58.2 g, 245.2 mmol), Pd₂(dba)₃ (6.7 g, 7.4 mmol), P(t-Bu)₃ (3.0 g, 14.7 mmol), NaOt-Bu (47.1 g, 490.4 mmol), toluene (1226 mL) were added to a round-bottomed flask in the same manner as Sub 1-1 to obtain 73.0 g of the product. (Yield: 71.3%)

### 3) Synthesis of Sub 2-5

Sub 2-5a (50.0 g, 209.7 mmol) was placed in a round-bottom flask, dissolved in THF (1048 mL) and Sub 2-5b (34.4 g, 209.7 mmol), Pd(PPh₃)₄ (14.5 g, 12.6 mmol), NaOH (25.2 g, 629.1 mmol), and water (524 ml) were added and the reaction was carried out at 80° C. When the reaction was complete, the reaction product was extracted with CH₂Cl₂ and water, dried with MgSO₄, concentrated, and the resulting compound was recrystallized using a silica gel column to obtain 39.9 g of the product. (Yield: 82.4%)

### 4) Synthesis of P-17

Sub 1-16 (50.0 g, 119.7 mmol), Sub 2-5 (27.6 g, 119.7 mmol), Pd₂(dba)₃ (3.3 g, 3.6 mmol), P(t-Bu)₃ (1.5 g, 7.2 mmol), NaOt-Bu (23.0 g, 239.5 mmol), toluene (599 mL) were added to a round-bottomed flask in the same manner as Sub 1-1 to obtain 51.9 g of the product. (Yield: 70.8%)

### 3. Synthesis of P-25

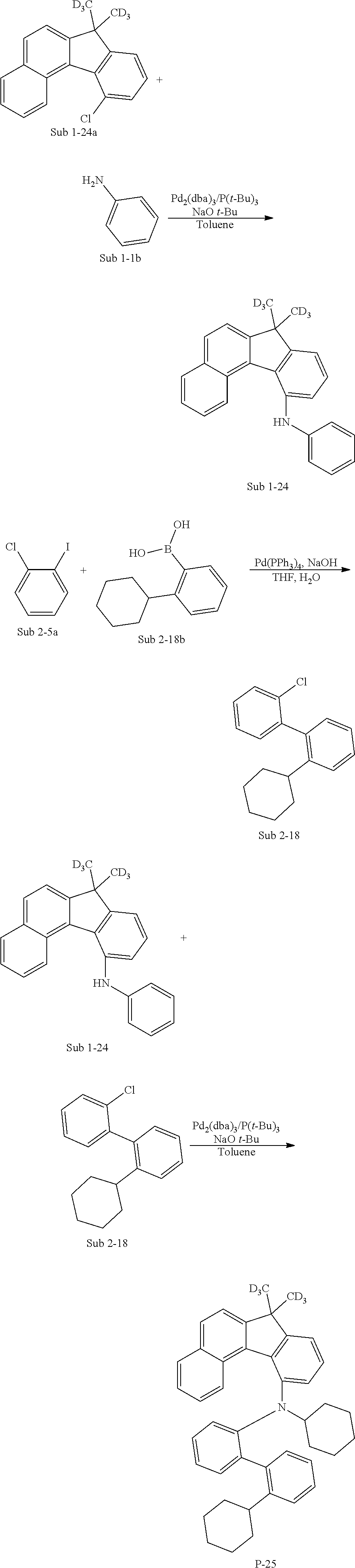

### 1) Synthesis of Sub 1-24

Sub 1-24a (100.0 g, 351.1 mmol), Sub 1-1b (32.7 g, 351.1 mmol), Pd₂(dba)₃ (9.7 g, 10.5 mmol), P(t-Bu)₃ (4.3 g, 21.1 mmol), NaOt-Bu (67.5 g, 702.2 mmol), toluene (1755 mL) were added to a round-bottomed flask in the same manner as Sub 1-1 to obtain 84.8 g of the product. (Yield: 70.7%)

### 2) Synthesis of Sub 2-18

Sub 2-5a (50.0 g, 209.7 mmol) was placed in a round-bottom flask, dissolved in THF (1048 mL) and Sub 2-18b (42.8 g, 209.7 mmol), Pd(PPh₃)₄ (14.5 g, 12.6 mmol), NaOH (25.2 g, 629.1 mmol), and water (524 ml) were added, and the same experiment as Sub 2-5 was performed to obtain 46.2 g of the product. (Yield: 81.3%)

### 3) Synthesis of P-25

Sub 1-24 (50.0 g, 146.4 mmol), Sub 2-18 (39.6 g, 146.4 mmol), Pd₂(dba)₃ (4.0 g, 4.4 mmol), P(t-Bu)₃ (1.8 g, 8.8 mmol), NaOt-Bu (28.1 g, 292.8 mmol), toluene (732 mL) were added to a round-bottomed flask in the same manner as Sub 1-1 to obtain 58.8 g of the product. (Yield: 69.8%)

### 4. Synthesis of P-44

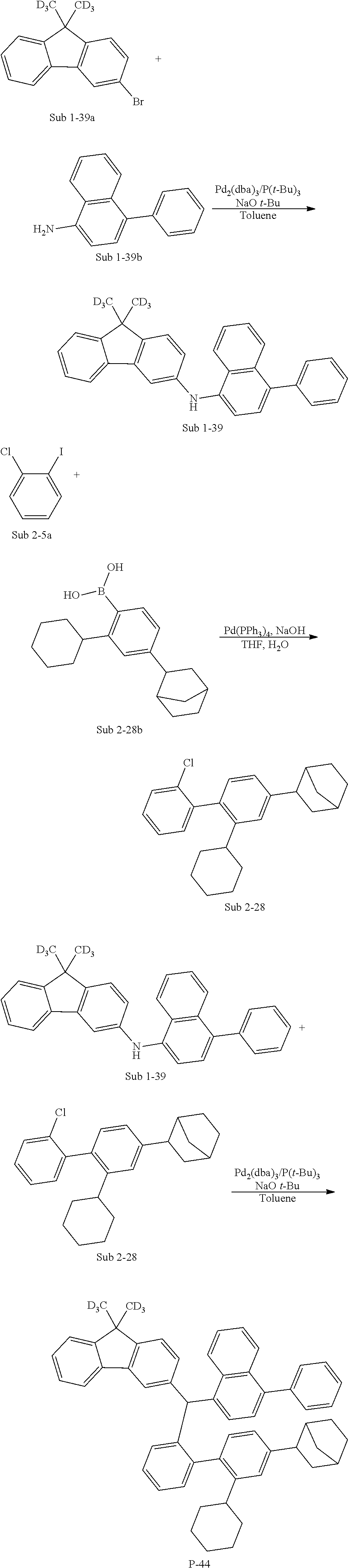

### 1) Synthesis of Sub 1-39

Sub 1-39a (100.0 g, 358.2 mmol), Sub 1-39b (78.5 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 106.9 g of the product. (Yield: 71.5%)

### 2) Synthesis of Sub 2-28

Sub 2-5a (50.0 g, 209.7 mmol) was placed in a round-bottom flask, dissolved in THF (1048 mL) and Sub 2-28b (62.5 g, 209.7 mmol), Pd(PPh₃)₄ (14.5 g, 12.6 mmol), NaOH (25.2 g, 629.1 mmol), and water (524 ml) were added, and the same experiment as Sub 2-5 was performed to obtain 61.8 g of the product. (Yield: 80.8%)

### 3) Synthesis of P-44

Sub 1-39 (50.0 g, 119.7 mmol), Sub 2-28 (43.7 g, 119.7 mmol), Pd₂(dba)₃ (3.3 g, 3.6 mmol), P(t-Bu)₃ (1.5 g, 7.2 mmol), NaOt-Bu (23.0 g, 239.5 mmol), toluene (599 mL) were added to a round-bottomed flask in the same manner as Sub 1-1 to obtain 62.7 g of the product. (Yield: 70.2%)

### 5. Synthesis of P-61

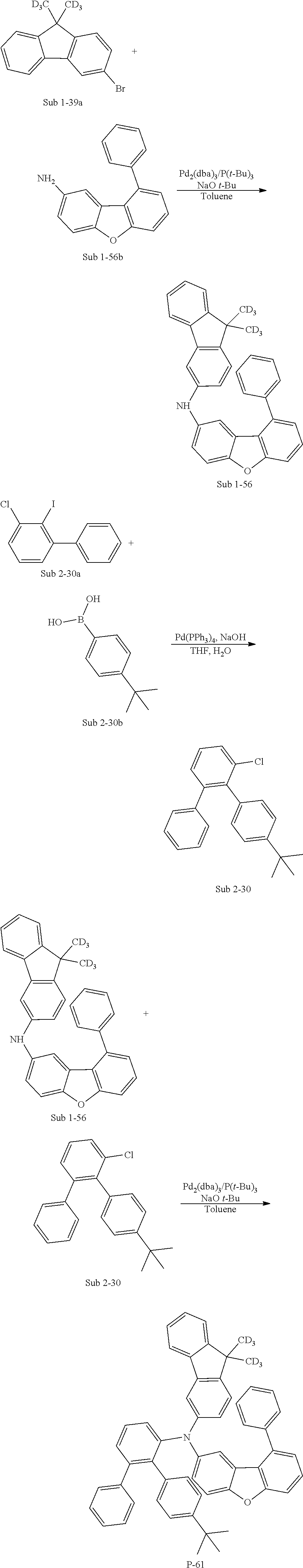

### 1) Synthesis of Sub 1-56

Sub 1-39a (100.0 g, 358.2 mmol), Sub 1-56b (92.9 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 116.4 g of the product. (Yield: 71.0%)

### 2) Synthesis of Sub 2-30

Sub 2-30a (50.0 g, 159.0 mmol) was placed in a round-bottom flask, dissolved in THF (795 mL) and Sub 2-30b (28.3 g, 159.0 mmol), Pd(PPh₃)₄ (11.0 g, 9.5 mmol), NaOH (19.1 g, 476.9 mmol), and water (397 ml) were added, and the same experiment as Sub 2-5 was performed to obtain 39.5 g of the product. (Yield: 77.4%)

### 3) Synthesis of P-61

Sub 1-56 (50.0 g, 109.3 mmol), Sub 2-30 (35.1 g, 109.3 mmol), Pd₂(dba)₃ (3.0 g, 3.3 mmol), P(t-Bu)₃ (1.3 g, 6.6 mmol), NaOt-Bu (21.0 g, 218.5 mmol), toluene (546 mL) were added to a round-bottomed flask in the same manner as Sub 1-1 to obtain 57.0 g of the product. (Yield: 69.9%)

### 6. Synthesis of P-84

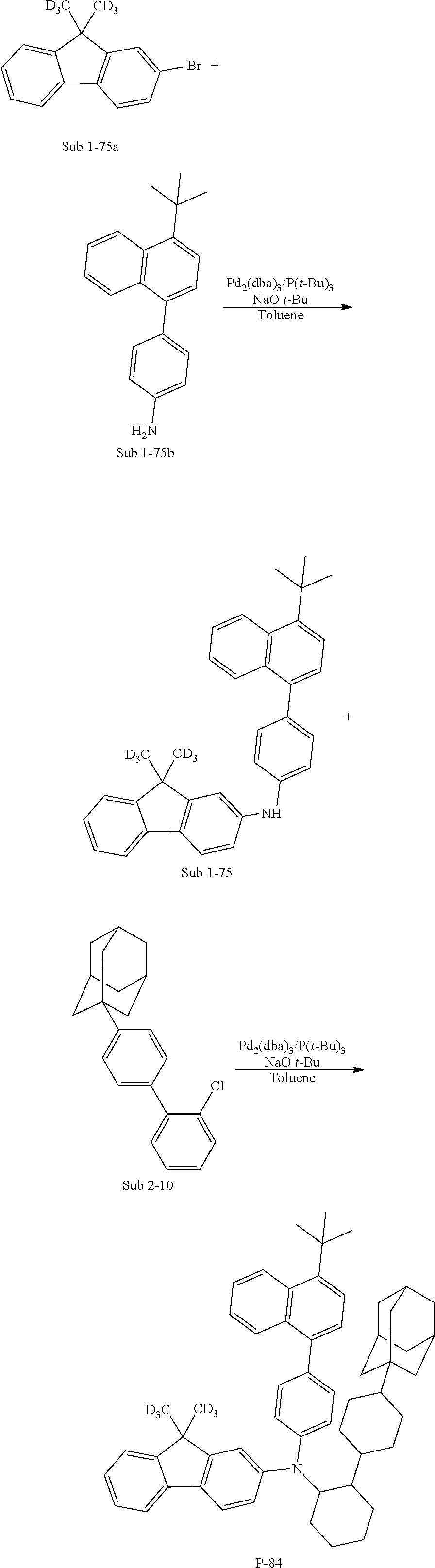

### 1) Synthesis of Sub 1-75

Sub 1-75a (100.0 g, 358.2 mmol), Sub 1-75b (98.6 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 122.3 g of the product. (Yield: 72.1%)

### 2) Synthesis of P-84

Sub 1-75 (50.0 g, 109.3 mmol), Sub 2-10 (35.3 g, 109.3 mmol), Pd₂(dba)₃ (3.0 g, 3.3 mmol), P(t-Bu)₃ (1.3 g, 6.6 mmol), NaOt-Bu (21.0 g, 218.5 mmol), toluene (546 mL) were added to a round-bottomed flask in the same manner as Sub 1-1 to obtain 61.0 g of the product. (Yield: 73.5%)

### 7. Synthesis of P-89

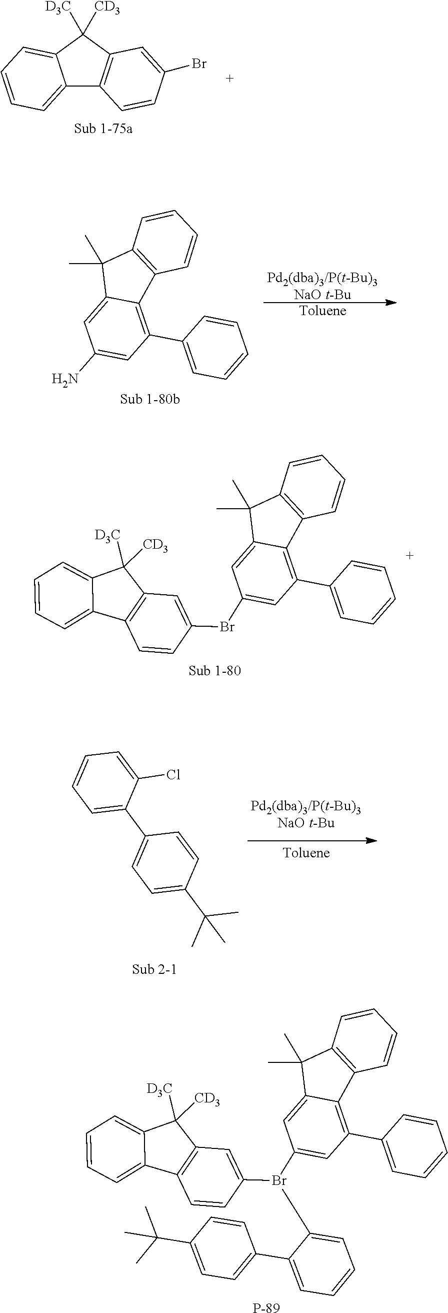

### 1) Synthesis of Sub 1-80

Sub 1-75a (100.0 g, 358.2 mmol), Sub 1-80b (102.2 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 124.2 g of the product. (Yield: 72.7%)

### 2) Synthesis of P-89

Sub 1-80 (50.0 g, 103.4 mmol), Sub 2-1 (25.3 g, 103.4 mmol), Pd₂(dba)₃ (2.8 g, 3.1 mmol), P(t-Bu)₃ (1.3 g, 6.2 mmol), NaOt-Bu (19.9 g, 206.7 mmol), toluene (517 mL) were added to a round-bottomed flask in the same manner as Sub 1-1 to obtain 51.6 g of the product. (Yield: 72.1%)

### 8. Synthesis of P-92

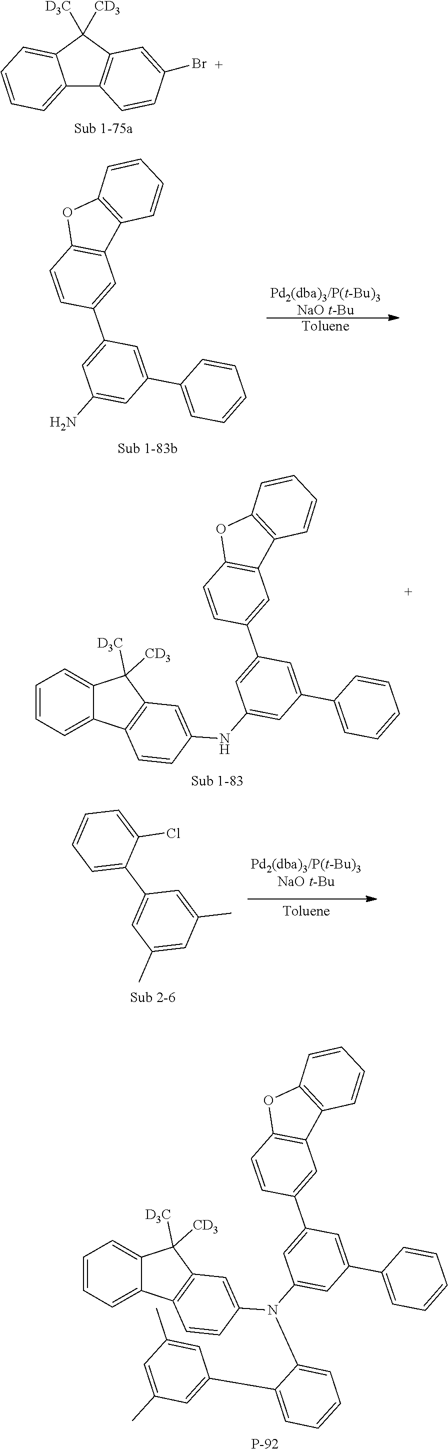

### 1) Synthesis of Sub 1-83

Sub 1-75a (100.0 g, 358.2 mmol), Sub 1-83b (127.3 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 138.4 g of the product. (Yield: 72.4%)

### 2) Synthesis of P-92

Sub 1-83 (50.0 g, 93.7 mmol), Sub 2-6 (20.3 g, 93.7 mmol), Pd₂(dba)₃ (2.6 g, 2.8 mmol), P(t-Bu)₃ (1.1 g, 5.6 mmol), NaOt-Bu (18.0 g, 187.4 mmol), toluene (468 mL) were added to a round-bottomed flask in the same manner as Sub 1-1 to obtain 46.4 g of the product. (Yield: 69.4%)

### 9. Synthesis of P-115

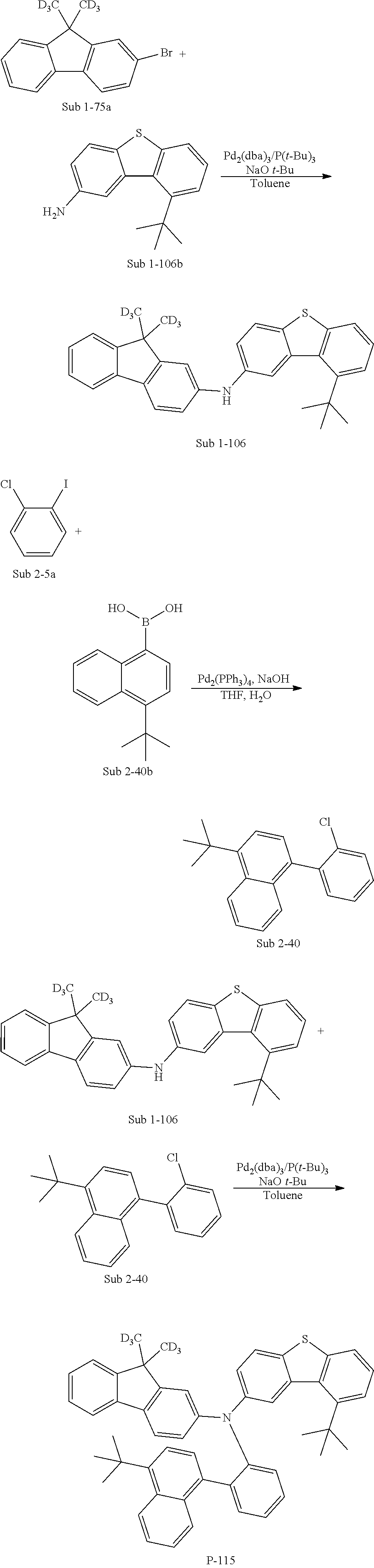

### 1) Synthesis of Sub 1-106

Sub 1-75a (100.0 g, 358.2 mmol), Sub 1-106b (91.5 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 114.7 g of the product. (Yield: 70.6%)

### 2) Synthesis of Sub 2-40

Sub 2-5a (50.0 g, 209.7 mmol) was placed in a round-bottom flask, dissolved in THE (1048 ml) and Sub 2-40b (47.8 g, 209.7 mmol), Pd(PPh₃)₄ (14.5 g, 12.6 mmol), NaOH (25.2 g, 629.1 mmol) and water (524 ml) were added, and the same experiment as Sub 2-5 was performed to obtain 50.2 g of the product. (Yield: 81.2%)

### 3) Synthesis of P-115

Sub 1-106 (50.0 g, 110.2 mmol), Sub 2-40 (32.5 g, 110.2 mmol), Pd₂(dba)₃ (3.0 g, 3.3 mmol), P(t-Bu)₃ (1.3 g, 6.6 mmol), NaOt-Bu (21.2 g, 220.4 mmol), toluene (551 mL) were added to a round-bottomed flask in the same manner as Sub 1-1 to obtain 55.0 g of the product. (Yield: 70.1%)

### 10. Synthesis of P-126

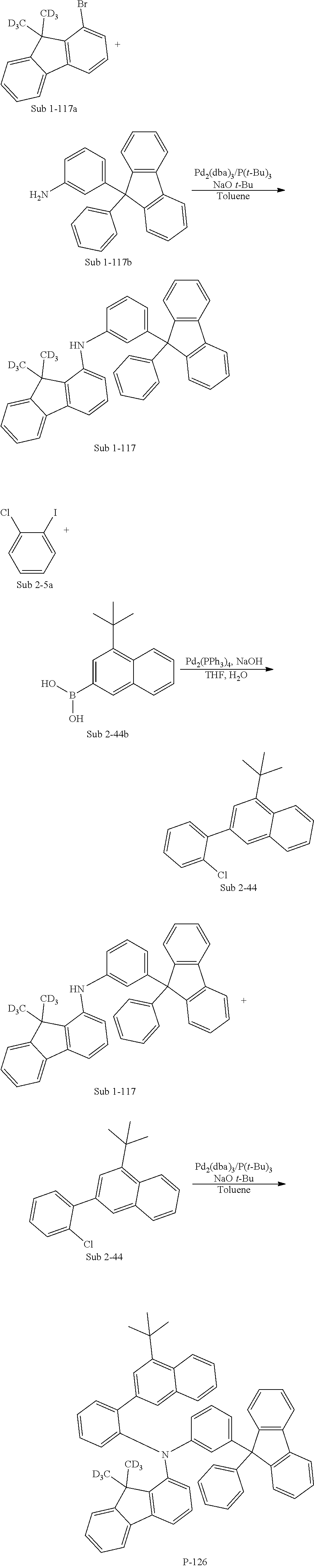

### 1) Synthesis of Sub 1-117

Sub 1-117a (100.0 g, 358.2 mmol), Sub 1-117b (91.5 g, 358.2 mmol), Pd₂(dba)₃ (9.8 g, 10.7 mmol), P(t-Bu)₃ (4.4 g, 21.5 mmol), NaOt-Bu (68.8 g, 716.3 mmol), toluene (1791 mL) were added to a round-bottomed flask in the same manner as Sub 1-1 to obtain 134.8 g of the product. (Yield: 70.8%)

### 2) Synthesis of Sub 2-44

Sub 2-5a (50.0 g, 209.7 mmol) was placed in a round-bottom flask, dissolved in THE (1048 ml) and Sub 2-40b (47.8 g, 209.7 mmol), Pd(PPh₃)₄ (14.5 g, 12.6 mmol), NaOH (25.2 g, 629.1 mmol) and water (524 ml) were added, and the same experiment as Sub 2-5 was performed to obtain 50.3 g of the product. (Yield: 81.3%)

### 3) Synthesis of P-126

Sub 1-117 (50.0 g, 94.0 mmol), Sub 2-44 (21.4 g, 94.0 mmol), Pd₂(dba)₃ (2.6 g, 2.8 mmol), P(t-Bu)₃ (1.1 g, 5.6 mmol), NaOt-Bu (18.1 g, 188.1 mmol), toluene (470 mL) were added to a round-bottomed flask in the same manner as Sub 1-1 to obtain 51.0 g of the product. (Yield: 68.7%)

Sub 1 of the reaction scheme 1 may be, but is not limited to, the compounds below, and the FD-MS (Field Desorption-Mass Spectrometry) values of compounds belonging to Sub 1 are as shown in Table 1.

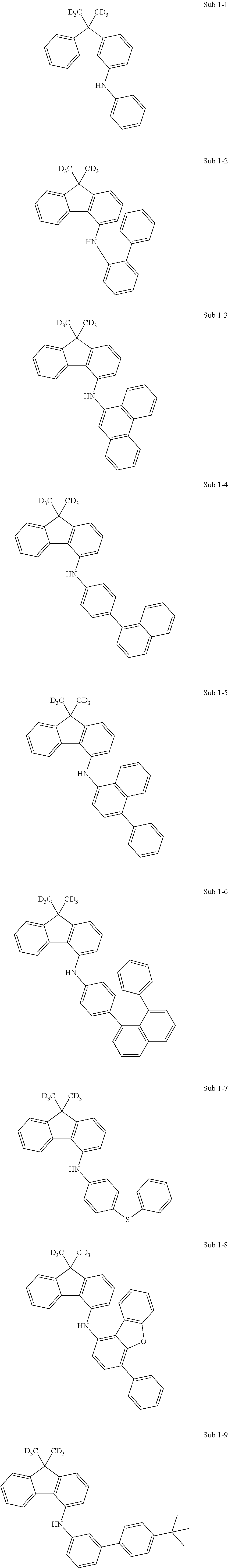

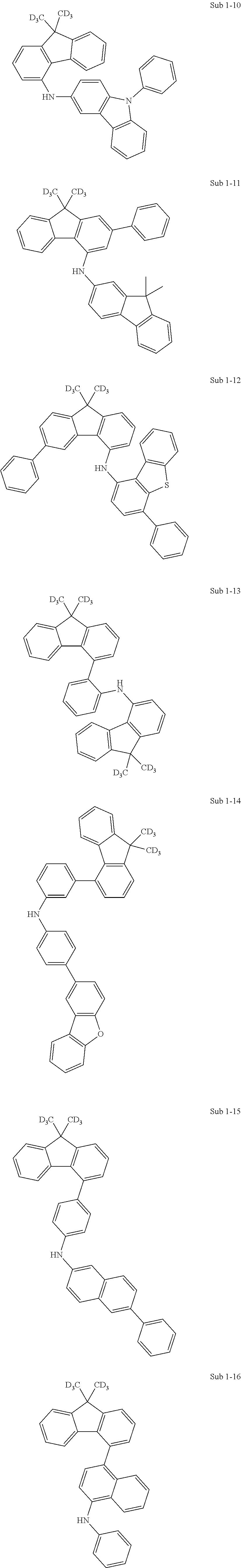

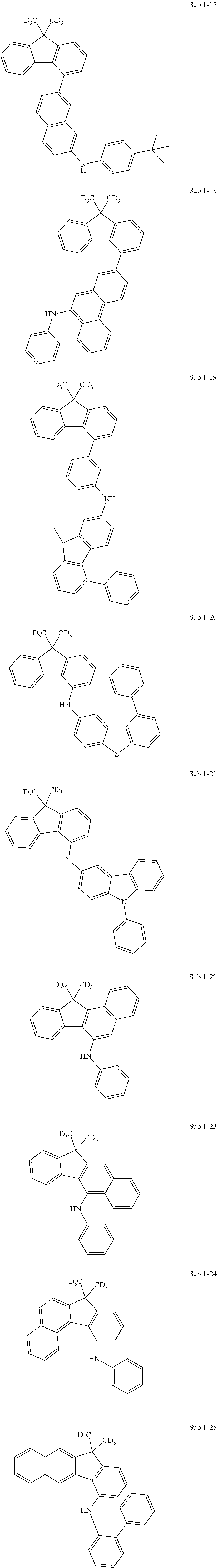

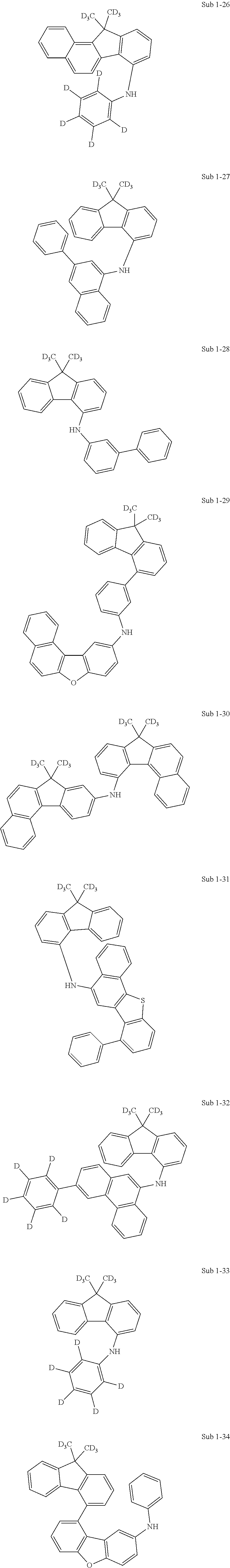

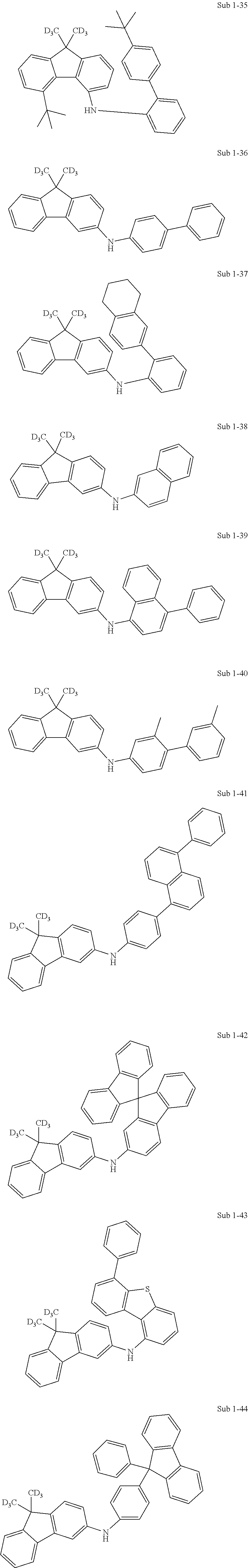

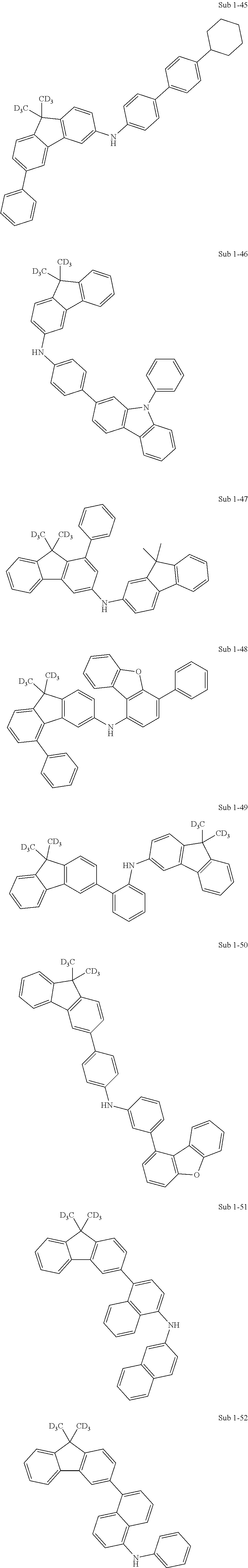

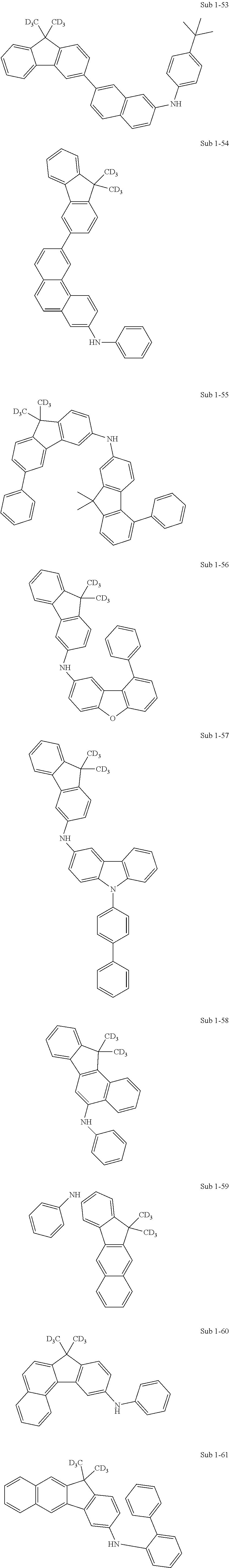

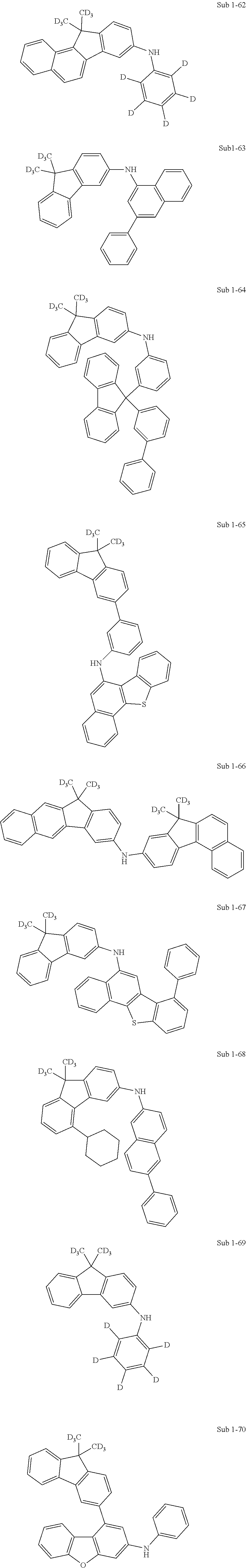

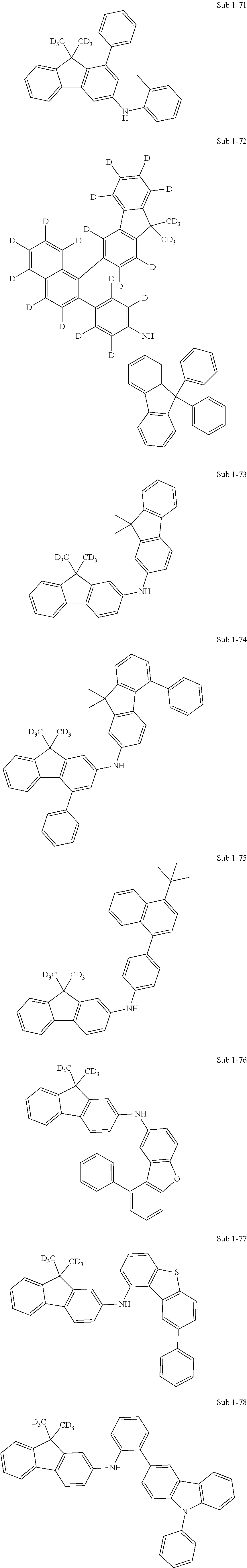

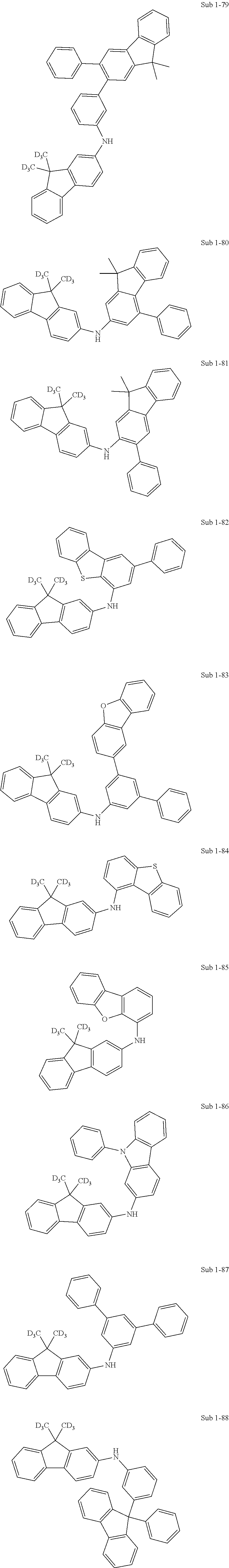

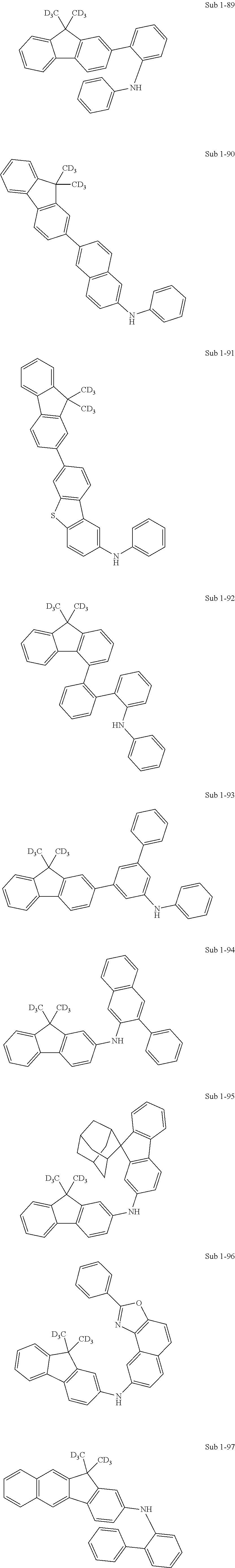

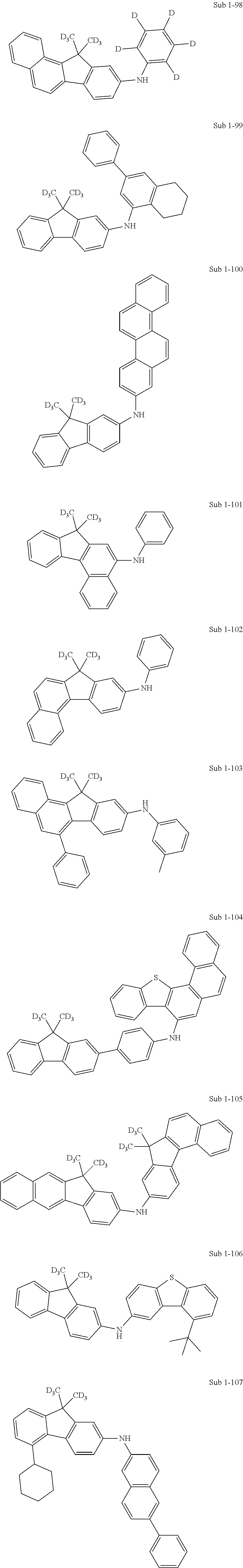

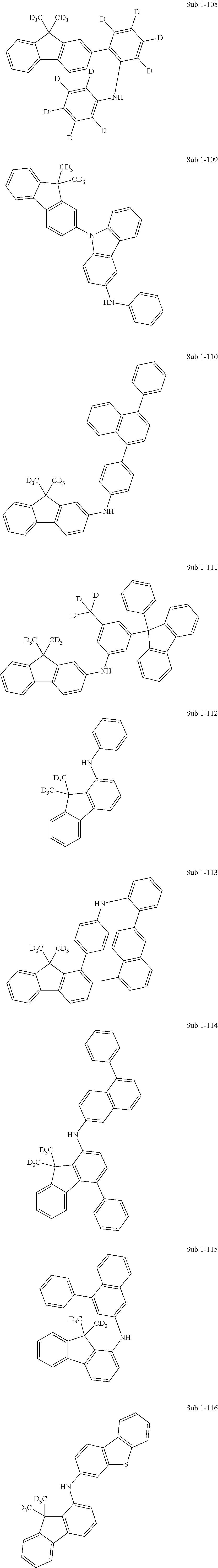

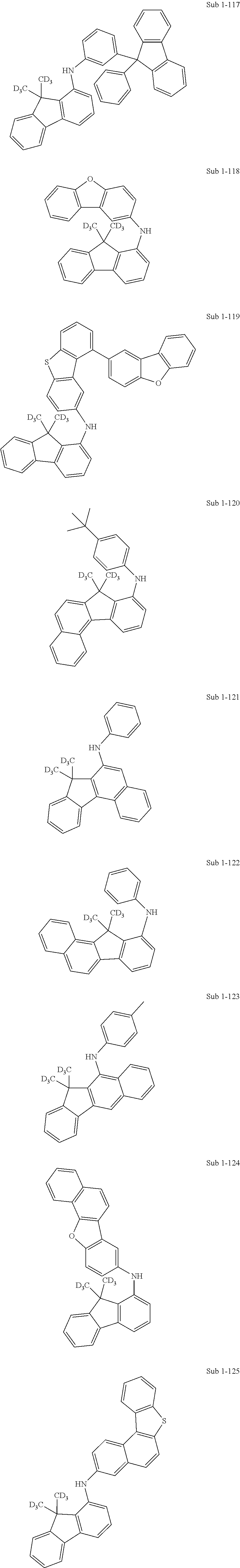

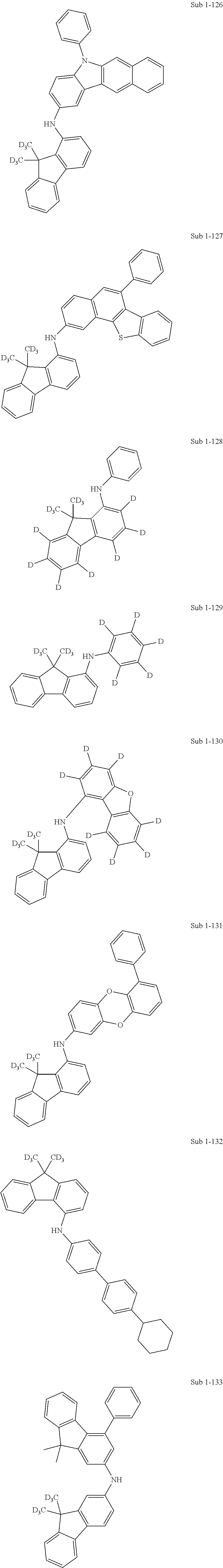

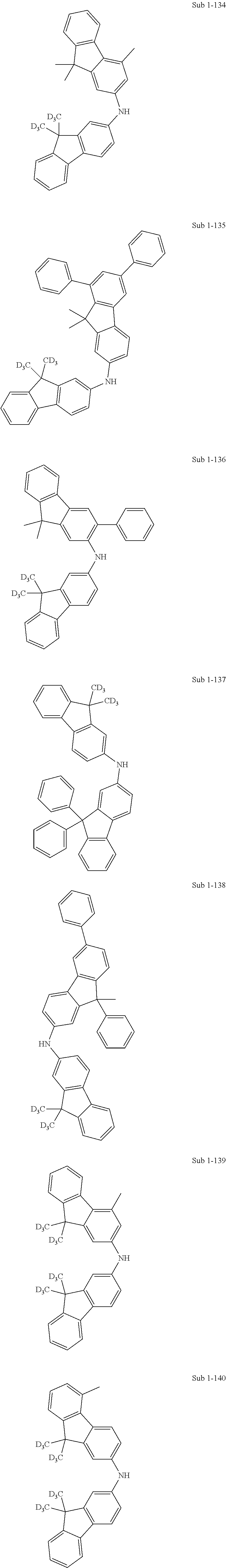

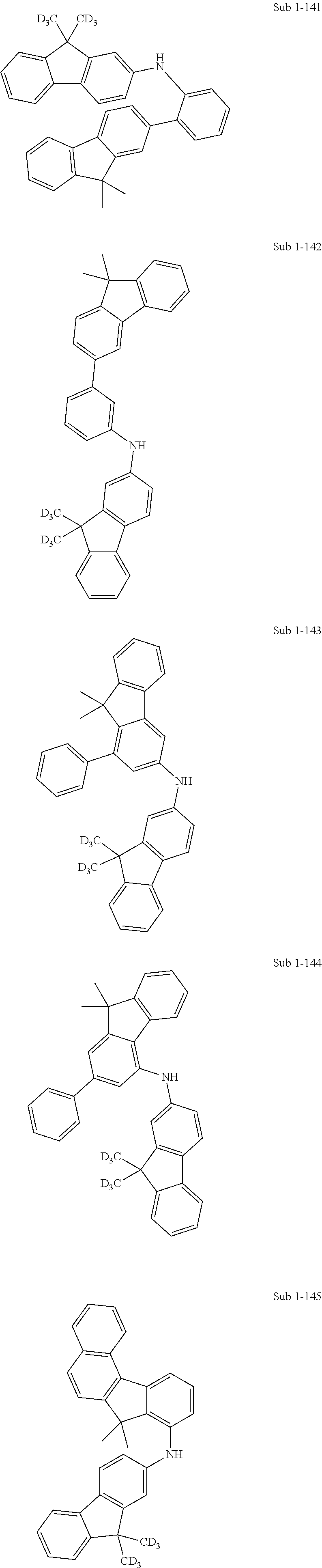 [SMILES: [2H]C([2H])([2H])C1(C([2H])([2H])[2H])c2ccccc2-c2ccc(Nc3ccccc3-c3ccc4c(c3)C(C)(C)c3ccccc3-4)cc21 ; [2H]C([2H])([2H])C1(C([2H])([2H])[2H])c2ccccc2-c2ccc(Nc3cccc(-c4ccc5c(c4)-c4ccccc4C5(C)C)c3)cc21 ; [2H]C([2H])([2H])C1(C([2H])([2H])[2H])c2ccccc2-c2ccc(Nc3cc(-c4ccccc4)c4c(c3)-c3ccccc3C4(C)C)cc21 ; [2H]C([2H])([2H])C1(C([2H])([2H])[2H])c2ccccc2-c2ccc(Nc3cc(-c4ccccc4)cc4c3-c3ccccc3C4(C)C)cc21 ; [2H]C([2H])([2H])C1(C([2H])([2H])[2H])c2ccccc2-c2ccc(Nc3cccc4c3C(C)(C)c3ccc5ccccc5c3-4)cc21]

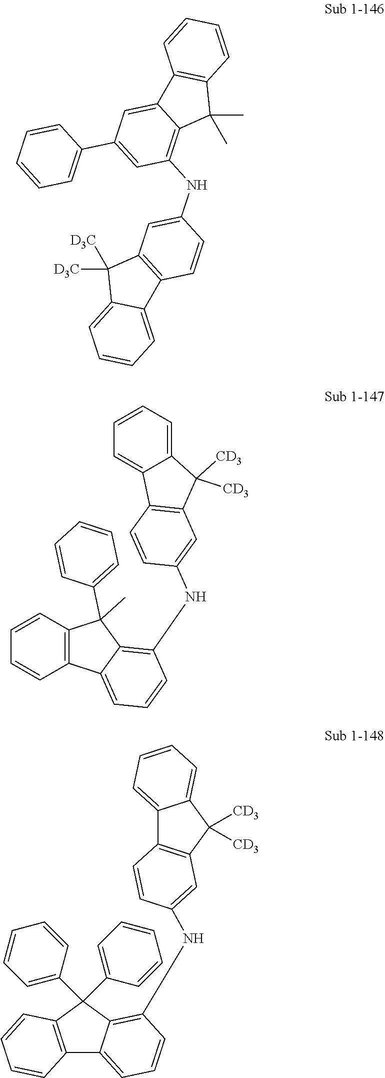

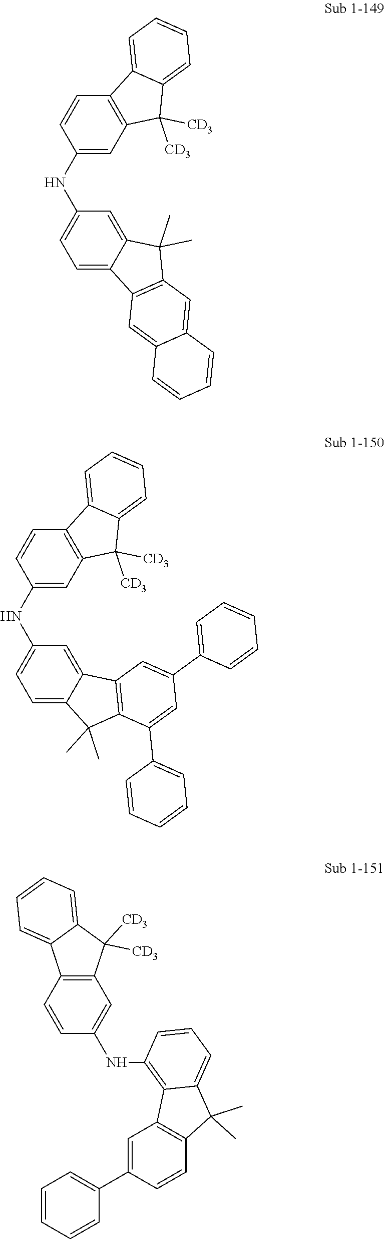 [SMILES: [2H]C([2H])([2H])C1(C([2H])([2H])[2H])c2ccccc2-c2ccc(Nc3ccc4c(c3)C(C)(C)c3cc5ccccc5cc3-4)cc21 ; [2H]C([2H])([2H])C1(C([2H])([2H])[2H])c2ccccc2-c2ccc(Nc3ccc4c(c3)-c3cc(-c5ccccc5)cc(-c5ccccc5)c3C4(C)C)cc21 ; [2H]C([2H])([2H])C1(C([2H])([2H])[2H])c2ccccc2-c2ccc(Nc3cccc4c3-c3cc(-c5ccccc5)ccc3C4(C)C)cc21]

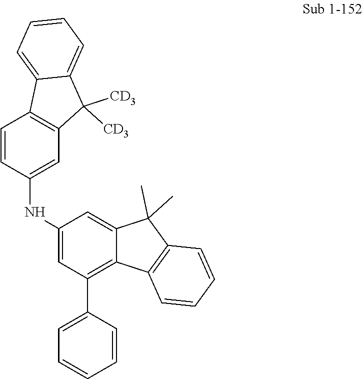 [SMILES: [2H]C([2H])([2H])C1(C([2H])([2H])[2H])c2ccccc2-c2ccc(Nc3cc(-c4ccccc4)c4c(c3)C(C)(C)c3ccccc3-4)cc21]

TABLE 1

| Compound | FD-MS | Compound | FD-MS |
|---|---|---|---|
| Sub 1-1 | m/z = 291.19(C₂₁H₁₃D₆N = 291.43) | Sub 1-2 | m/z = 367.22(C₂₇H₁₇D₆N = 367.52) |
| Sub 1-3 | m/z = 391.22(C₂₉H₁₇D₆N = 391.55) | Sub 1-4 | m/z = 417.24(C₃₁H₁₉D₆N = 417.58) |
| Sub 1-5 | m/z = 417.24(C₃₁H₁₉D₆N = 417.58) | Sub 1-6 | m/z = 493.27(C₃₇H₂₃D₆N = 493.68) |
| Sub 1-7 | m/z = 397.18(C₂₇H₁₅D₆NS = 397.57) | Sub 1-8 | m/z = 457.23(C₃₃H₁₉D₆NO = 457.61) |
| Sub 1-9 | m/z = 423.28(C₃₁H₂₅D₆N = 423.63) | Sub 1-10 | m/z = 456.25(C₃₃H₂₀D₆N₂ = 456.62) |
| Sub 1-11 | m/z = 483.28(C₃₆H₂₅D₆N = 483.69) | Sub 1-12 | m/z = 549.24(C₃₉H₂₃D₆NS = 549.76) |
| Sub 1-13 | m/z = 489.32(C₃₆H₁₉D₁₂N = 489.72) | Sub 1-14 | m/z = 533.26(C₃₉H₂₃D₆NO = 533.7) |
| Sub 1-15 | m/z = 493.27(C₃₇H₂₃D₆N = 493.68) | Sub 1-16 | m/z = 417.24(C₃₁H₁₉D₆N = 417.58) |
| Sub 1-17 | m/z = 473.30(C₃₅H₂₇D₆N = 473.69) | Sub 1-18 | m/z = 467.25(C₃₅H₂₁D₆N = 467.64) |
| Sub 1-19 | m/z = 559.31(C₄₂H₂₉D₆N = 559.79) | Sub 1-20 | m/z = 473.21(C₃₃H₁₉D₆NS = 473.67) |
| Sub 1-21 | m/z = 456.25(C₃₃H₂₀D₆N₂ = 456.62) | Sub 1-22 | m/z = 341.21(C₂₅H₁₅D₆N = 341.49) |
| Sub 1-23 | m/z = 341.21(C₂₅H₁₅D₆N = 341.49) | Sub 1-24 | m/z = 341.21(C₂₅H₁₅D₆N = 341.49) |
| Sub 1-25 | m/z = 417.24(C₃₁H₁₉D₆N = 417.58) | Sub 1-26 | m/z = 346.24(C₂₅H₁₀D₁₁N = 346.52) |
| Sub 1-27 | m/z = 417.24(C₃₁H₁₉D₆N = 417.58) | Sub 1-28 | m/z = 367.22(C₂₇H₁₇D₆N = 367.52) |
| Sub 1-29 | m/z = 507.25(C₃₇H₂₁D₆NO = 507.67) | Sub 1-30 | m/z = 513.32(C₃₈H₁₉D₁₂N = 513.75) |
| Sub 1-31 | m/z = 523.22(C₃₇H₂₁D₆NS = 523.73) | Sub 1-32 | m/z = 472.28(C₃₅H₁₆D₁₁N = 472.68) |
| Sub 1-33 | m/z = 296.22(C₂₁H₈D₁₁N = 296.46) | Sub 1-34 | m/z = 457.23(C₃₃H₁₉D₆NO = 457.61) |
| Sub 1-35 | m/z = 479.35(C₃₅H₃₃D₆N = 479.74) | Sub 1-36 | m/z = 367.22(C₂₇H₁₇D₆N = 367.52) |
| Sub 1-37 | m/z = 421.27(C₃₁H₂₃D₆N = 421.62) | Sub 1-38 | m/z = 341.21(C₂₅H₁₅D₆N = 341.49) |
| Sub 1-39 | m/z = 417.24(C₃₁H₁₉D₆N = 417.58) | Sub 1-40 | m/z = 395.25(C₂₉H₂₁D₆N = 395.58) |
| Sub 1-41 | m/z = 493.27(C₃₇H₂₃D₆N = 493.68) | Sub 1-42 | m/z = 529.27(C₄₀H₂₃D₆N = 529.72) |
| Sub 1-43 | m/z = 473.21(C₃₃H₁₉D₆NS = 473.67) | Sub 1-44 | m/z = 531.28(C₄₀H₂₅D₆N = 531.73) |
| Sub 1-45 | m/z = 525.33(C₃₉H₃₁D₆N = 525.77) | Sub 1-46 | m/z = 532.28(C₃₉H₂₄D₆N₂ = 532.72) |
| Sub 1-47 | m/z = 483.28(C₃₆H₂₅D₆N = 483.69) | Sub 1-48 | m/z = 533.26(C₃₉H₂₃D₆NO = 533.70) |
| Sub 1-49 | m/z = 489.32(C₃₆H₁₉D₁₂N = 489.72) | Sub 1-50 | m/z = 533.26(C₃₉H₂₃D₆NO = 533.70) |
| Sub 1-51 | m/z = 467.25(C₃₅H₂₁D₆N = 467.64) | Sub 1-52 | m/z = 417.24(C₃₁H₁₉D₆N = 417.58) |
| Sub 1-53 | m/z = 473.30(C₃₅H₂₇D₆N = 473.69) | Sub 1-54 | m/z = 467.25(C₃₅H₂₁D₆N = 467.64) |
| Sub 1-55 | m/z = 559.31(C₄₂H₂₉D₆N = 559.79) | Sub 1-56 | m/z = 457.23(C₃₃H₁₉D₆NO = 457.61) |
| Sub 1-57 | m/z = 532.28(C₃₉H₂₄D₆N₂ = 532.72) | Sub 1-58 | m/z = 341.21(C₂₅H₁₅D₆N = 341.49) |
| Sub 1-59 | m/z = 341.21(C₂₅H₁₅D₆N = 341.49) | Sub 1-60 | m/z = 341.21(C₂₅H₁₅D₆N = 341.49) |
| Sub 1-61 | m/z = 417.24(C₃₁H₁₉D₆N = 417.58) | Sub 1-62 | m/z = 346.24(C₂₅H₁₀D₁₁N = 346.52) |
| Sub 1-63 | m/z = 417.24(C₃₁H₁₉D₆N = 417.58) | Sub 1-64 | m/z = 607.31(C₄₆H₂₉D₆N = 607.83) |
| Sub 1-65 | m/z = 523.22(C₃₇H₂₁D₆NS = 523.73) | Sub 1-66 | m/z = 513.32(C₃₈H₁₉D₁₂N = 513.75) |
| Sub 1-67 | m/z = 523.22(C₃₇H₂₁D₆NS = 523.73) | Sub 1-68 | m/z = 499.31(C₃₇H₂₉D₆N = 499.73) |
| Sub 1-69 | m/z = 296.22(C₂₁H₈D₁₁N = 296.46) | Sub 1-70 | m/z = 457.23(C₃₃H₁₉D₆NO = 457.61) |
| Sub 1-71 | m/z = 381.24(C₂₈H₁₉D₆N = 381.55) | Sub 1-72 | m/z = 750.47(C₅₆H₁₈D₂₃N = 751.09) |
| Sub 1-73 | m/z = 407.25(C₃₀H₂₁D₆N = 407.59) | Sub 1-74 | m/z = 559.31(C₄₂H₂₉D₆N = 559.79) |
| Sub 1-75 | m/z = 473.3(C₃₅H₂₇D₆N = 473.69) | Sub 1-76 | m/z = 457.23(C₃₃H₁₉D₆NO = 457.61) |
| Sub 1-77 | m/z = 473.21(C₃₃H₁₉D₆NS = 473.67) | Sub 1-78 | m/z = 532.28(C₃₉H₂₄D₆N₂ = 532.72) |
| Sub 1-79 | m/z = 559.31(C₄₂H₂₉D₆N = 559.79) | Sub 1-80 | m/z = 483.28(C₃₆H₂₅D₆N = 483.69) |
| Sub 1-81 | m/z = 483.28(C₃₆H₂₅D₆N = 483.69) | Sub 1-82 | m/z = 473.21(C₃₃H₁₉D₆NS = 473.67) |
| Sub 1-83 | m/z = 533.26(C₃₉H₂₃D₆NO = 533.70) | Sub 1-84 | m/z = 397.18(C₂₇H₁₅D₆NS = 397.57) |
| Sub 1-85 | m/z = 381.2(C₂₇H₁₅D₆NO = 381.51) | Sub 1-86 | m/z = 456.25(C₃₃H₂₀D₆N₂ = 456.62) |
| Sub 1-87 | m/z = 443.25(C₃₃H₂₁D₆N = 443.62) | Sub 1-88 | m/z = 531.28(C₄₀H₂₅D₆N = 531.73) |
| Sub 1-89 | m/z = 367.22(C₂₇H₁₇D₆N = 367.52) | Sub 1-90 | m/z = 417.24(C₃₁H₁₉D₆N = 417.58) |
| Sub 1-91 | m/z = 473.21(C₃₃H₁₉D₆NS = 473.67) | Sub 1-92 | m/z = 443.25(C₃₃H₂₁D₆N = 443.62) |
| Sub 1-93 | m/z = 443.25(C₃₃H₂₁D₆N = 443.62) | Sub 1-94 | m/z = 417.24(C₃₁H₁₉D₆N = 417.58) |
| Sub 1-95 | m/z = 499.31(C₃₇H₂₉D₆N = 499.73) | Sub 1-96 | m/z = 458.23(C₃₂H₁₈D₆N₂O = 458.59) |
| Sub 1-97 | m/z = 417.24(C₃₁H₁₉D₆N = 417.58) | Sub 1-98 | m/z = 346.24(C₂₅H₁₀D₁₁N = 346.52) |
| Sub 1-99 | m/z = 421.27(C₃₁H₂₃D₆N = 421.62) | Sub 1-100 | m/z = 441.24(C₃₃H₁₉D₆N = 441.61) |
| Sub 1-101 | m/z = 341.21(C₂₅H₁₅D₆N = 341.49) | Sub 1-102 | m/z = 341.21(C₂₅H₁₅D₆N = 341.49) |
| Sub 1-103 | m/z = 431.25(C₃₂H₂₁D₆N = 431.61) | Sub 1-104 | m/z = 573.24(C₄₁H₂₃D₆NS = 573.79) |
| Sub 1-105 | m/z = 513.32(C₃₈H₁₉D₁₂N = 513.75) | Sub 1-106 | m/z = 453.24(C₃₁H₂₃D₆NS = 453.68) |
| Sub 1-107 | m/z = 499.31(C₃₇H₂₉D₆N = 499.73) | Sub 1-108 | m/z = 376.28(C₂₇H₈D₁₅N = 376.58) |
| Sub 1-109 | m/z = 456.25(C₃₃H₂₀D₆N₂ = 456.62) | Sub 1-110 | m/z = 493.27(C₃₇H₂₃D₆N = 493.68) |
| Sub 1-111 | m/z = 548.32(C₄₁H₂₄D₉N = 548.78) | Sub 1-112 | m/z = 291.19(C₂₁H₁₃D₆N = 291.43) |
| Sub 1-113 | m/z = 507.28(C₃₈H₂₅D₆N = 507.71) | Sub 1-114 | m/z = 493.27(C₃₇H₂₃D₆N = 493.68) |
| Sub 1-115 | m/z = 417.24(C₃₁H₁₉D₆N = 417.58) | Sub 1-116 | m/z = 397.18(C₂₇H₁₅D₆NS = 397.57) |
| Sub 1-117 | m/z = 531.28(C₄₀H₂₅D₆N = 531.73) | Sub 1-118 | m/z = 381.20(C₂₇H₁₅D₆NO = 381.51) |
| Sub 1-119 | m/z = 563.22(C₃₉H₂₁D₆NOS = 563.75) | Sub 1-120 | m/z = 397.27(C₂₉H₂₃D₆N = 397.59) |
| Sub 1-121 | m/z = 341.21(C₂₅H₁₅D₆N = 341.49) | Sub 1-122 | m/z = 341.21(C₂₅H₁₅D₆N = 341.49) |
| Sub 1-123 | m/z = 355.22(C₂₆H₁₇D₆N = 355.51) | Sub 1-124 | m/z = 431.22(C₃₁H₁₇D₆NO = 431.57) |
| Sub 1-125 | m/z = 447.19(C₃₁H₁₇D₆NS = 447.63) | Sub 1-126 | m/z = 506.26(C₃₇H₂₂D₆N₂ = 506.68) |
| Sub 1-127 | m/z = 523.22(C₃₇H₂₁D₆NS = 523.73) | Sub 1-128 | m/z = 298.23(C₂₁H₆D₁₃N = 298.47) |
| Sub 1-129 | m/z = 296.22(C₂₁H₈D₁₁N = 296.46) | Sub 1-130 | m/z = 388.24(C₂₇H₈D₁₃NO = 388.55) |
| Sub 1-131 | m/z = 473.23(C₃₃H₁₉D₆NO₂ = 473.6) | Sub 1-132 | m/z = 449.3(C₃₃H₂₇D₆N = 449.67) |
| Sub 1-133 | m/z = 483.28(C₃₆H₂₅D₆N = 483.69) | Sub 1-134 | m/z = 421.27(C₃₁H₂₃D₆N = 421.62) |
| Sub 1-135 | m/z = 559.31(C₄₂H₂₉D₆N = 559.79) | Sub 1-136 | m/z = 483.28(C₃₆H₂₅D₆N = 483.69) |
| Sub 1-137 | m/z = 531.28(C₄₀H₂₅D₆N = 531.73) | Sub 1-138 | m/z = 545.3(C₄₁H₂₇D₆N = 545.76) |
| Sub 1-139 | m/z = 427.31(C₃₁H₁₇D₁₂N = 427.65) | Sub 1-140 | m/z = 427.31(C₃₁H₁₇D₁₂N = 427.65) |
| Sub 1-141 | m/z = 483.28(C₃₆H₂₅D₆N = 483.69) | Sub 1-142 | m/z = 483.28(C₃₆H₂₅D₆N = 483.69) |
| Sub 1-143 | m/z = 483.28(C₃₆H₂₅D₆N = 483.69) | Sub 1-144 | m/z = 483.28(C₃₆H₂₅D₆N = 483.69) |
| Sub 1-145 | m/z = 457.27(C₃₄H₂₃D₆N = 457.65) | Sub 1-146 | m/z = 483.28(C₃₆H₂₅D₆N = 483.69) |
| Sub 1-147 | m/z = 469.27(C₃₅H₂₃D₆N = 469.66) | Sub 1-148 | m/z = 531.28(C₄₀H₂₅D₆N = 531.73) |
| Sub 1-149 | m/z = 457.27(C₃₄H₂₃D₆N = 457.65) | Sub 1-150 | m/z = 559.31(C₄₂H₂₉D₆N = 559.79) |
| Sub 1-151 | m/z = 483.28(C₃₆H₂₅D₆N = 483.69) | Sub 1-152 | m/z = 483.28(C₃₆H₂₅D₆N = 483.69) |

Sub 2 of the reaction scheme 1 may be, but is not limited to, the compounds below, and the FD-MS (Field Desorption-Mass Spectrometry) values of compounds belonging to Sub 2 are as shown in Table 2.

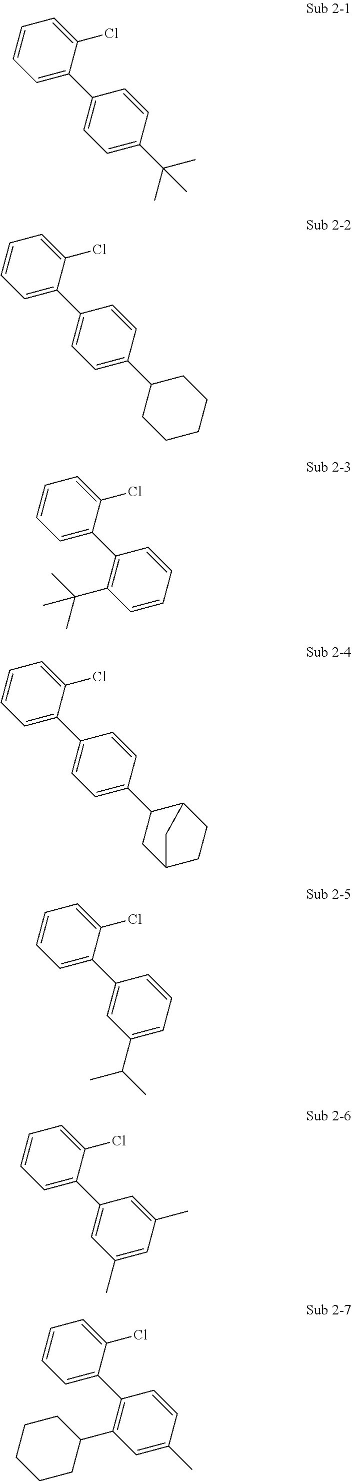

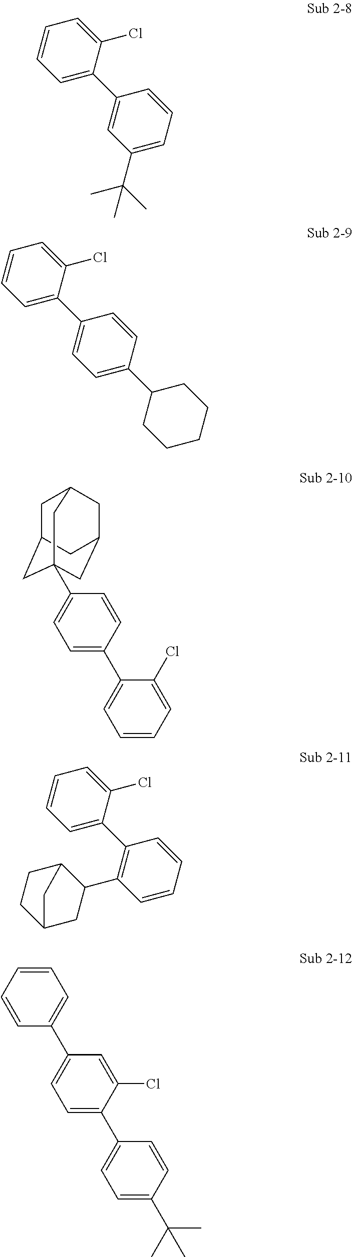

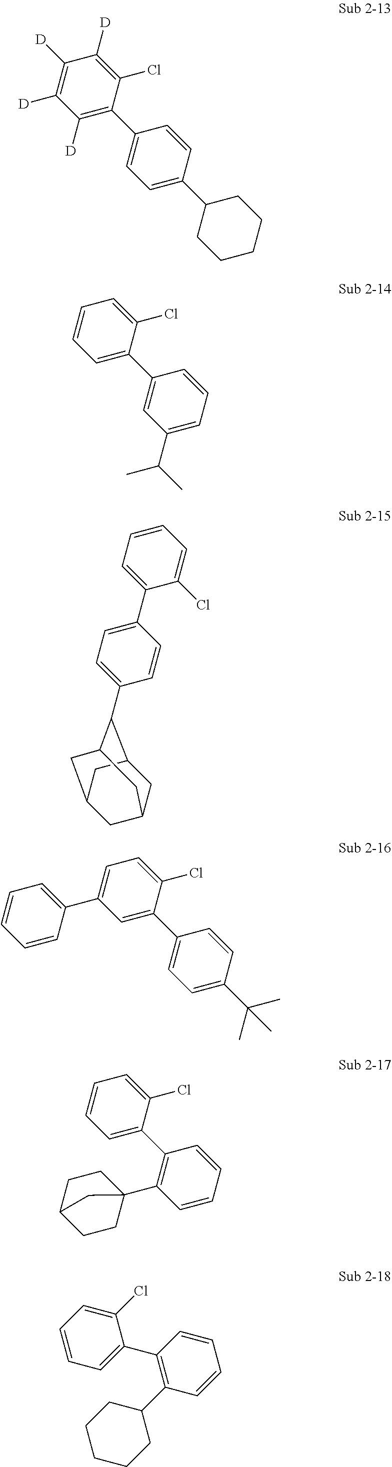

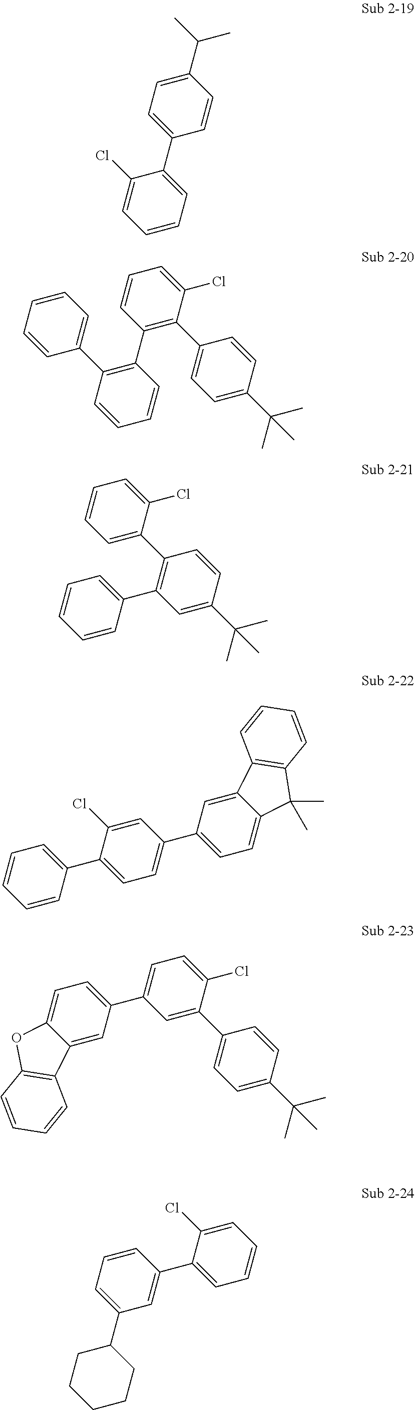

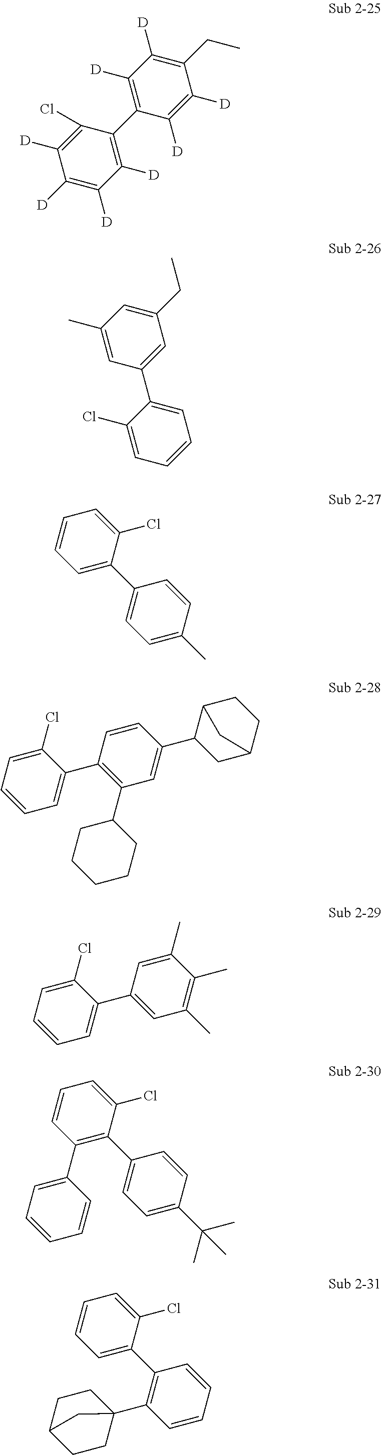

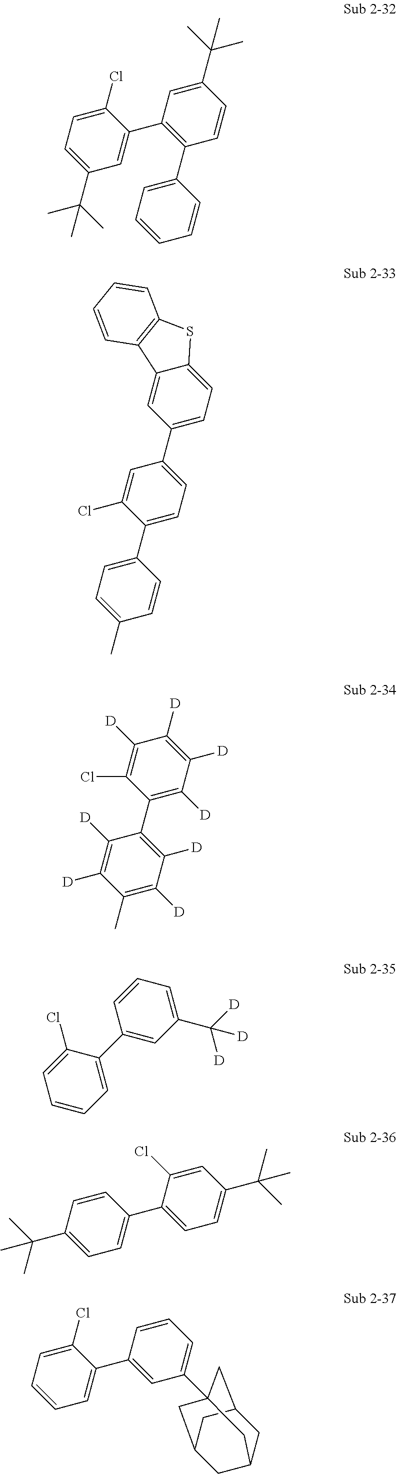

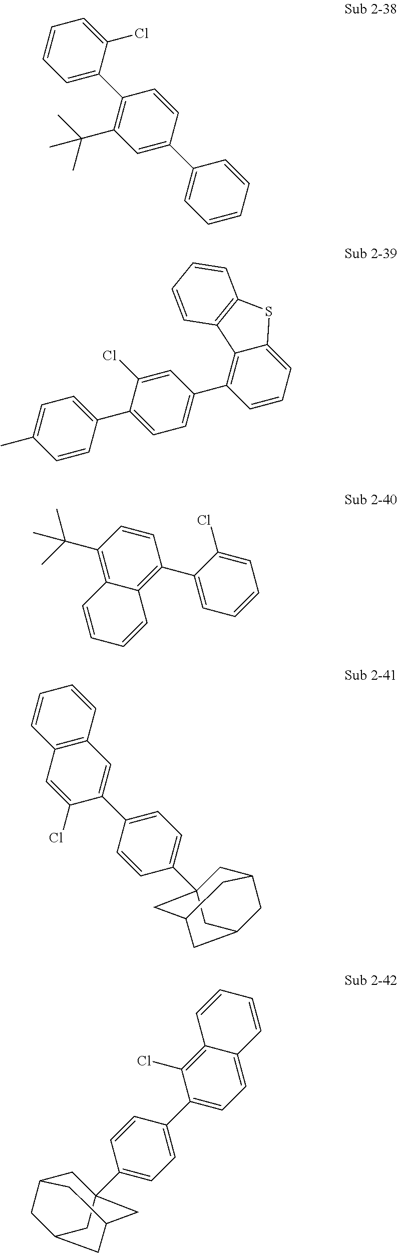

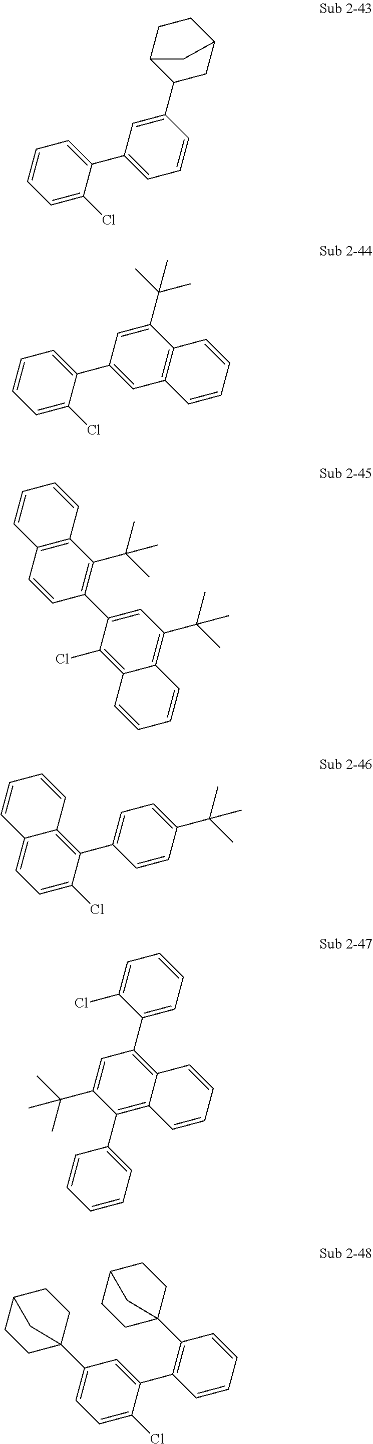

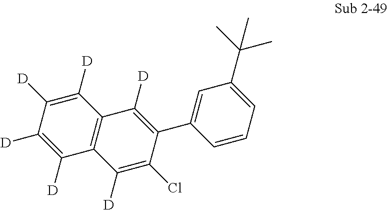 [SMILES: [2H]c1c([2H])c([2H])c2c([2H])c(-c3cccc(C(C)(C)C)c3)c(Cl)c([2H])c2c1[2H]]

TABLE 2

| Compound | FD-MS | Compound | FD-MS |
|---|---|---|---|
| Sub 2-1 | m/z = 244.10(C₁₆H₁₇Cl = 244.76) | Sub 2-2 | m/z = 270.12(C₁₈H₁₉Cl = 270.80) |
| Sub 2-3 | m/z = 244.10(C₁₆H₁₇Cl = 244.76) | Sub 2-4 | m/z = 282.12(C₁₉H₁₉Cl = 282.81) |
| Sub 2-5 | m/z = 230.09(C₁₅H₁₅Cl = 230.74) | Sub 2-6 | m/z = 216.07(C₁₄H₁₃Cl = 216.71) |
| Sub 2-7 | m/z = 284.13(C₁₉H₂₁Cl = 284.83) | Sub 2-8 | m/z = 244.10(C₁₆H₁₇Cl = 244.76) |
| Sub 2-9 | m/z = 270.12(C₁₈H₁₉Cl = 270.80) | Sub 2-10 | m/z = 322.15(C₂₂H₂₃Cl = 322.88) |
| Sub 2-11 | m/z = 282.12(C₁₉H₁₉Cl = 282.81) | Sub 2-12 | m/z = 320.13(C₂₂H₂₁Cl = 320.86) |
| Sub 2-13 | m/z = 320.13(C₂₂H₂₁Cl = 320.86) | Sub 2-14 | m/z = 230.09(C₁₅H₁₅Cl = 230.74) |
| Sub 2-15 | m/z = 322.15(C₂₂H₂₃Cl = 322.88) | Sub 2-16 | m/z = 320.13(C₂₂H₂₁Cl = 320.86) |
| Sub 2-17 | m/z = 282.12(C₁₉H₁₉Cl = 282.81) | Sub 2-18 | m/z = 270.12(C₁₈H₁₉Cl = 270.80) |
| Sub 2-19 | m/z = 230.09(C₁₅H₁₅Cl = 230.74) | Sub 2-20 | m/z = 396.16(C₂₈H₂₅Cl = 396.96) |
| Sub 2-21 | m/z = 320.13(C₂₂H₂₁Cl = 320.86) | Sub 2-22 | m/z = 394.15(C₂₈H₂₃Cl = 394.94) |
| Sub 2-23 | m/z = 410.14(C₂₈H₂₃ClO = 410.94) | Sub 2-24 | m/z = 270.12(C₁₈H₁₉Cl = 270.80) |
| Sub 2-25 | m/z = 224.12(C₁₄H₅D₈Cl = 224.76) | Sub 2-26 | m/z = 230.09(C₁₅H₁₅Cl = 230.74) |
| Sub 2-27 | m/z = 202.05(C₁₃H₁₁Cl = 202.68) | Sub 2-28 | m/z = 364.20(C₂₅H₂₉Cl = 364.96) |
| Sub 2-29 | m/z = 230.09(C₁₅H₁₅Cl = 230.74) | Sub 2-30 | m/z = 320.13(C₂₂H₂₁Cl = 320.86) |
| Sub 2-31 | m/z = 282.12(C₁₉H₁₉Cl = 282.81) | Sub 2-32 | m/z = 376.20(C₂₆H₂₉Cl = 376.97) |
| Sub 2-33 | m/z = 384.07(C₂₅H₁₇ClS = 384.92) | Sub 2-34 | m/z = 210.11(C₁₃H₃D₈Cl = 210.73) |
| Sub 2-35 | m/z = 205.07(C₁₃H₈D₃Cl = 205.70) | Sub 2-36 | m/z = 300.16(C₂₀H₂₅Cl = 300.87) |
| Sub 2-37 | m/z = 322.15(C₂₂H₂₃Cl = 322.88) | Sub 2-38 | m/z = 320.13(C₂₂H₂₁Cl = 320.86) |
| Sub 2-39 | m/z = 384.07(C₂₅H₁₇ClS = 384.92) | Sub 2-40 | m/z = 294.12(C₂₀H₁₉Cl = 294.82) |
| Sub 2-41 | m/z = 372.16(C₂₆H₂₅Cl = 372.94) | Sub 2-42 | m/z = 372.16(C₂₆H₂₅Cl = 372.94) |
| Sub 2-43 | m/z = 282.12(C₁₉H₁₉Cl = 282.81) | Sub 2-44 | m/z = 294.12(C₂₀H₁₉Cl = 294.82) |
| Sub 2-45 | m/z = 400.20(C₂₈H₂₉Cl = 400.99) | Sub 2-46 | m/z = 294.12(C₂₀H₁₉Cl = 294.82) |
| Sub 2-47 | m/z = 370.15(C₂₆H₂₃Cl = 370.92) | Sub 2-48 | m/z = 376.20(C₂₆H₂₉Cl = 376.97) |
| Sub 2-49 | m/z = 300.16(C₂₀H₁₃D₆Cl = 300.86) | | |

The ED-MS (Field Desorption-Mass Spectrometry) values of compounds P-1 to P-160 of the present invention manufactured according to the above synthetic examples are as shown in Table 3.

TABLE 3

| Compound | FD-MS | Compound | FD-MS |
|---|---|---|---|
| P-1 | m/z = 499.31(C₃₇H₂₉D₆N = 499.73) | P-2 | m/z = 601.36(C₄₅H₃₅D₆N = 601.87) |
| P-3 | m/z = 599.35(C₄₅H₃₃D₆N = 599.85) | P-4 | m/z = 663.38(C₅₀H₃₇D₆N = 663.94) |
| P-5 | m/z = 611.35(C₄₆H₃₃D₆N = 611.86) | P-6 | m/z = 673.36(C₅₁H₃₅D₆N = 673.93) |
| P-7 | m/z = 645.33(C₄₆H₃₅D₆NS = 645.94) | P-8 | m/z = 665.36(C₄₉H₃₅D₆NO = 665.91) |
| P-9 | m/z = 631.41(C₄₇H₄₁D₆N = 631.94) | P-10 | m/z = 683.44(C₅₁H₄₅D₆N = 684.01) |
| P-11 | m/z = 742.42(C₅₅H₄₂D₆N₂ = 743.04) | P-12 | m/z = 729.42(C₅₅H₄₃D₆N = 730.04) |
| P-13 | m/z = 835.41(C₆₁H₄₅D₆NS = 836.18) | P-14 | m/z = 697.45(C₅₂H₃₅D₁₂N = 698.03) |
| P-15 | m/z = 817.42(C₆₁H₄₃D₆NO = 818.11) | P-16 | m/z = 731.43(C₅₅H₃₇D₁₀N = 732.05) |
| P-17 | m/z = 611.35(C₄₆H₃₃D₆N = 611.86) | P-18 | m/z = 681.42(C₅₁H₄₃D₆N = 682.00) |
| P-19 | m/z = 753.42(C₅₇H₄₃D₆N = 754.06) | P-20 | m/z = 767.44(C₅₈H₄₅D₆N = 768.09) |
| P-21 | m/z = 757.36(C₅₅H₃₉D₆NS = 758.07) | P-22 | m/z = 702.39(C₅₂H₃₈D₆N₂ = 702.97) |
| P-23 | m/z = 549.33(C₄₁H₃₁D₆N = 549.79) | P-24 | m/z = 549.33(C₄₁H₃₁D₆N = 549.79) |
| P-25 | m/z = 575.35(C₄₃H₃₃D₆N = 575.83) | P-26 | m/z = 625.36(C₄₇H₃₅D₆N = 625.89) |
| P-27 | m/z = 540.35(C₄₀H₂₄D₁₁N = 540.79) | P-28 | m/z = 703.41(C₅₃H₄₁D₆N = 704.00) |
| P-29 | m/z = 651.38(C₄₉H₃₇D₆N = 651.93) | P-30 | m/z = 651.38(C₄₉H₃₇D₆N = 651.93) |
| P-31 | m/z = 649.36(C₄₉H₃₅D₆N = 649.91) | P-32 | m/z = 741.39(C₅₅H₃₉D₆NO = 742.01) |
| P-33 | m/z = 715.37(C₅₃H₃₇D₆NO = 715.97) | P-34 | m/z = 747.46(C₅₆H₃₇D₁₂N = 748.09) |
| P-35 | m/z = 731.35(C₅₃H₃₇D₆NS = 732.03) | P-36 | m/z = 758.46(C₅₇H₃₈D₁₁N = 759.09) |
| P-37 | m/z = 484.36(C₃₅H₁₂D₁₉N = 484.76) | P-38 | m/z = 651.34(C₄₈H₃₃D₆NO = 651.88) |
| P-39 | m/z = 687.47(C₅₁H₄₉D₆N = 688.04) | P-40 | m/z = 563.26(C₄₀H₂₅D₆NS = 563.79) |
| P-41 | m/z = 575.35(C₄₃H₃₃D₆N = 575.83) | P-42 | m/z = 655.41(C₄₉H₄₁D₆N = 655.96) |
| P-43 | m/z = 549.33(C₄₁H₃₁D₆N = 549.79) | P-44 | m/z = 745.46(C₅₆H₄₇D₆N = 746.08) |
| P-45 | m/z = 589.36(C₄₄H₃₅D₆N = 589.86) | P-46 | m/z = 687.38(C₅₂H₃₇D₆N = 687.96) |
| P-47 | m/z = 763.41(C₅₈H₄₁D₆N = 764.06) | P-48 | m/z = 681.33(C₄₉H₃₅D₆NS = 681.97) |
| P-49 | m/z = 739.41(C₅₆H₄₁D₆N = 740.04) | P-50 | m/z = 691.41(C₅₂H₄₁D₆N = 691.99) |
| P-51 | m/z = 818.45(C₆₁H₄₆D₆N₂ = 819.14) | P-52 | m/z = 729.42(C₅₅H₄₃D₆N = 730.04) |
| P-53 | m/z = 819.43(C₆₁H₄₅D₆NO = 820.12) | P-54 | m/z = 697.45(C₅₂H₃₅D₁₂N = 698.03) |
| P-55 | m/z = 741.39(C₅₅H₃₉D₆NO = 742.01) | P-56 | m/z = 705.42(C₅₃H₃₅D₁₀N = 706.01) |
| P-57 | m/z = 611.35(C₄₆H₃₃D₆N = 611.86) | P-58 | m/z = 681.42(C₅₁H₄₃D₆N = 682.00) |
| P-59 | m/z = 753.42(C₅₇H₄₃D₆N = 754.06) | P-60 | m/z = 767.44(C₅₈H₄₅D₆N = 768.09) |
| P-61 | m/z = 741.39(C₅₅H₃₉D₆NO = 742.01) | P-62 | m/z = 778.42(C₅₈H₄₂D₆N₂ = 779.07) |
| P-63 | m/z = 549.33(C₄₁H₃₁D₆N = 549.79) | P-64 | m/z = 549.33(C₄₁H₃₁D₆N = 549.79) |
| P-65 | m/z = 575.35(C₄₃H₃₃D₆N = 575.83) | P-66 | m/z = 625.36(C₄₇H₃₅D₆N = 625.89) |
| P-67 | m/z = 580.38(C₄₃H₂₈D₁₁N = 580.86) | P-68 | m/z = 703.41(C₅₃H₄₁D₆N = 704.00) |
| P-69 | m/z = 651.38(C₄₉H₃₇D₆N = 651.93) | P-70 | m/z = 631.41(C₄₇H₄₁D₆N = 631.94) |
| P-71 | m/z = 639.29(C₄₆H₂₉D₆NS = 639.89) | P-72 | m/z = 815.44(C₆₂H₄₅D₆N = 816.13) |
| P-73 | m/z = 731.35(C₅₃H₃₇D₆NS = 732.03) | P-74 | m/z = 747.46(C₅₆H₃₇D₁₂N = 748.09) |
| P-75 | m/z = 731.35(C₅₃H₃₇D₆NS = 732.03) | P-76 | m/z = 785.49(C₅₉H₅₁D₆N = 786.15) |
| P-77 | m/z = 470.35(C₃₄H₁₀D₁₉N = 470.73) | P-78 | m/z = 626.33(C₄₆H₂₆D₉NO = 626.85) |
| P-79 | m/z = 589.36(C₄₄H₃₅D₆N = 589.86) | P-80 | m/z = 958.59(C₇₂H₃₄D₂₃N = 959.4) |
| P-81 | m/z = 575.35(C₄₃H₃₃D₆N = 575.83) | P-82 | m/z = 641.39(C₄₈H₃₉D₆N = 641.93) |
| P-83 | m/z = 767.44(C₅₈H₄₅D₆N = 768.09) | P-84 | m/z = 759.47(C₅₇H₄₉D₆N = 760.11) |
| P-85 | m/z = 703.37(C₅₂H₃₇D₆NO = 703.96) | P-86 | m/z = 681.33(C₄₉H₃₅D₆NS = 681.97) |
| P-87 | m/z = 740.40(C₅₅H₄₀D₆N₂ = 741.02) | P-88 | m/z = 725.39(C₅₅H₃₉D₆N = 726.01) |
| P-89 | m/z = 691.41(C₅₂H₄₁D₆N = 691.99) | P-90 | m/z = 769.46(C₅₈H₄₇D₆N = 770.11) |
| P-91 | m/z = 719.35(C₅₂H₃₇D₆NS = 720.02) | P-92 | m/z = 713.36(C₅₃H₃₅D₆NO = 713.95) |
| P-93 | m/z = 605.30(C₄₃H₃₁D₆NS = 605.87) | P-94 | m/z = 665.36(C₄₉H₃₅D₆NO = 665.91) |
| P-95 | m/z = 720.44(C₅₃H₄₄D₆N₂ = 721.03) | P-96 | m/z = 623.35(C₄₇H₃₃D₆N = 623.87) |
| P-97 | m/z = 765.42(C₅₈H₄₃D₆N = 766.07) | P-98 | m/z = 533.30(C₄₀H₂₇D₆N = 533.75) |
| P-99 | m/z = 703.41(C₅₃H₄₁D₆N = 704.00) | P-100 | m/z = 639.29(C₄₆H₂₉D₆NS = 639.89) |
| P-101 | m/z = 727.41(C₅₅H₄₁D₆N = 728.02) | P-102 | m/z = 651.38(C₄₉H₃₇D₆N = 651.93) |
| P-103 | m/z = 625.36(C₄₇H₃₅D₆N = 625.89) | P-104 | m/z = 707.44(C₅₃H₄₅D₆N = 708.03) |
| P-105 | m/z = 692.37(C₅₀H₃₆D₆N₂O = 692.94) | P-106 | m/z = 625.36(C₄₇H₃₅D₆N = 625.89) |
| P-107 | m/z = 580.38(C₄₃H₂₈D₁₁N = 580.86) | P-108 | m/z = 707.44(C₅₃H₄₅D₆N = 708.03) |
| P-109 | m/z = 649.36(C₄₉H₃₅D₆N = 649.91) | P-110 | m/z = 681.42(C₅₁H₄₃D₆N = 682.00) |
| P-111 | m/z = 689.30(C₅₀H₃₁D₆NS = 689.95) | P-112 | m/z = 639.38(C₄₈H₃₇D₆N = 639.92) |
| P-113 | m/z = 781.36(C₅₇H₃₉D₆NS = 782.09) | P-114 | m/z = 747.46(C₅₆H₃₇D₁₂N = 748.09) |
| P-115 | m/z = 711.38(C₅₁H₄₁D₆NS = 712.04) | P-116 | m/z = 835.5(C₆₃H₅₃D₆N = 836.21) |
| P-117 | m/z = 550.41(C₄₀H₁₀D₂₃N = 550.85) | P-118 | m/z = 625.34(C₄₆H₂₇D₉N₂ = 625.86) |
| P-119 | m/z = 701.39(C₅₃H₃₉D₆N = 701.99) | P-120 | m/z = 756.44(C₅₇H₄₀D₉N = 757.08) |
| P-121 | m/z = 499.31(C₃₇H₂₉D₆N = 499.73) | P-122 | m/z = 715.41(C₅₄H₄₁D₆N = 716.01) |
| P-123 | m/z = 727.41(C₅₅H₄₁D₆N = 728.02) | P-124 | m/z = 753.42(C₅₇H₄₃D₆N = 754.06) |
| P-125 | m/z = 643.32(C₄₆H₃₃D₆NS = 643.92) | P-126 | m/z = 789.42(C₆₀H₄₃D₆N = 790.10) |
| P-127 | m/z = 745.42(C₅₅H₄₃D₆NO = 746.04) | P-128 | m/z = 797.36(C₅₇H₃₉D₆NOS = 798.09) |
| P-129 | m/z = 563.35(C₄₂H₃₃D₆N = 563.82) | P-130 | m/z = 599.35(C₄₅H₃₃D₆N = 599.85) |
| P-131 | m/z = 675.38(C₅₁H₃₇D₆N = 675.95) | P-132 | m/z = 641.39(C₄₈H₃₉D₆N = 641.93) |
| P-133 | m/z = 639.34(C₄₇H₃₃D₆NO = 639.87) | P-134 | m/z = 681.33(C₄₉H₃₅D₆NS = 681.97) |
| P-135 | m/z = 740.40(C₅₅H₄₀D₆N₂ = 741.02) | P-136 | m/z = 863.44(C₆₃H₄₉D₆NS = 864.24) |
| P-137 | m/z = 506.36(C₃₇H₂₂D₁₃N = 506.77) | P-138 | m/z = 560.40(C₄₁H₂₀D₁₇N = 560.86) |
| P-139 | m/z = 674.42(C₄₉H₃₀D₁₃NO = 674.97) | P-140 | m/z = 681.35(C₄₉H₃₅D₆NO₂ = 681.91) |
| P-141 | m/z = 717.42(C₅₄H₄₃D₆N = 718.03) | P-142 | m/z = 587.35(C₄₄H₃₃D₆N = 587.84) |
| P-143 | m/z = 767.44(C₅₈H₄₅D₆N = 768.09) | P-144 | m/z = 769.46(C₅₈H₄₇D₆N = 770.11) |
| P-145 | m/z = 739.41(C₅₆H₄₁D₆N = 740.04) | P-146 | m/z = 711.38(C₅₄H₃₇D₆N = 711.98) |
| P-147 | m/z = 593.38(C₄₄H₂₇D₁₂N = 593.88) | P-148 | m/z = 593.38(C₄₄H₂₇D₁₂N = 593.88) |
| P-149 | m/z = 691.41(C₅₂H₄₁D₆N = 691.99) | P-150 | m/z = 652.38(C₄₉H₃₂D₉N = 652.93) |
| P-151 | m/z = 691.41(C₅₂H₄₁D₆N = 691.99) | P-152 | m/z = 649.36(C₄₉H₃₅D₆N = 649.91) |
| P-153 | m/z = 743.44(C₅₆H₄₅D₆N = 744.07) | P-154 | m/z = 691.41(C₅₂H₄₁D₆N = 691.99) |
| P-155 | m/z = 703.41(C₅₃H₄₁D₆N = 704) | P-156 | m/z = 725.39(C₅₅H₃₉D₆N = 726.01) |
| P-157 | m/z = 691.41(C₅₂H₄₁D₆N = 691.99) | P-158 | m/z = 793.46(C₆₀H₄₇D₆N = 794.13) |
| P-159 | m/z = 649.36(C₄₉H₃₅D₆N = 649.91) | P-160 | m/z = 677.39(C₅₁H₃₉D₆N = 677.96) |

In the above, exemplary synthesis examples of the present invention represented by Formula 1 have been described, but these are all based on the Buchwald-Hartwig cross coupling reaction, Miyaura boration reaction, Suzuki cross-coupling reaction, Intramolecular acid-induced cyclization reaction (J. mater. Chem. 1999, 9, 2095.), Pd(II)-catalyzed oxidative cyclization reaction (Org. Lett. 2011, 13, 5504), and PPh₃-mediated reductive cyclization reaction (J. Org. Chem. 2005, 70, 5014.), and it will be easily understood by those skilled in the art that the reaction proceeds even when other substituents defined in Formula 1 are bonded in addition to the substituents specified in the specific synthesis examples.

### Manufacturing Evaluation of Organic Electroluminescent Devices

### [Example 1] Red Organic Light Emitting Device (Emitting-Auxiliary Layer)

Compound A and Compound B were used on an ITO layer (anode) formed on a glass substrate, and Compound B was doped at a weight ratio of 98:2 to form a hole injection layer with a thickness of 10 nm. Then, Compound A was vacuum-deposited on the hole injection layer with a thickness of 110 nm to form a hole transport layer.

Next, the compound P-1 of the present invention was vacuum-deposited to a thickness of 10 nm on the hole transport layer to form an emitting auxiliary layer. Afterwards, compound D-R was used as the host material of the emitting layer, and bis-(1-phenylisoquinolyl)iridium(III)acetylacetonate (hereinafter abbreviated by ‘(piq)₂Ir(acac)’) was used as the dopant material, and the dopant was doped so that the weight ratio of the host and dopant was 95:5 to form an emitting layer with a thickness of 30 nm.

Next, compound E was vacuum-deposited on the emitting layer to form a hole blocking layer with a thickness of 10 nm, and a mixture of compound F and compound G at a weight ratio of 5:5 was used to form an electron transport layer with a thickness of 30 nm on the hole-blocking layer. Afterwards, compound G was deposited on the electron transport layer to form an electron injection layer with a thickness of 0.2 nm, and then Al was deposited to form a cathode with a thickness of 150 nm.
compound A: N-([1,1′-biphenyl]-4-yl)-9,9-dimethyl-N-(4-(9-phenyl-9H-carbazol-3-yl)phenyl)-9H-fluoren-2-amine
compound B: 4,4′,4″-((1E,1′E,1″E)-cyclopropane-1,2,3-triylidenetris(cyanomethaneylylidene))tris(2,3,5,6-tetrafluorobenzonitrile)
compound D-R: 14-(4-phenylquinazolin-2-yl)-14H-benzo[c]benzo[4,5]thieno[2,3-a]carbazole
compound E: 2-(4′-(9,9-dimethyl-9H-fluoren-2-yl)-[1,1′-biphenyl]-3-yl)-4,6-diphenyl-1,3,5-triazine
compound F: 2,7-bis(4-(4,6-diphenyl-1,3,5-triazin-2-yl)phenyl)naphthalene
compound G: (8-quinolinolato)lithium

### [Example 2] to [Example 35]

An organic light emitting device was manufactured in the same manner as in Example 1, except that the compound of the present invention described in Table 4 was used instead of the compound P-1 of the present invention as an emitting auxiliary layer material.

### [Comparative Example 1] and [Comparative Example 7]

An organic light emitting device was manufactured in the same manner as in Example 1, except that the comparative compounds A to G were used instead of the compound P-1 of the present invention as an emitting auxiliary layer material.

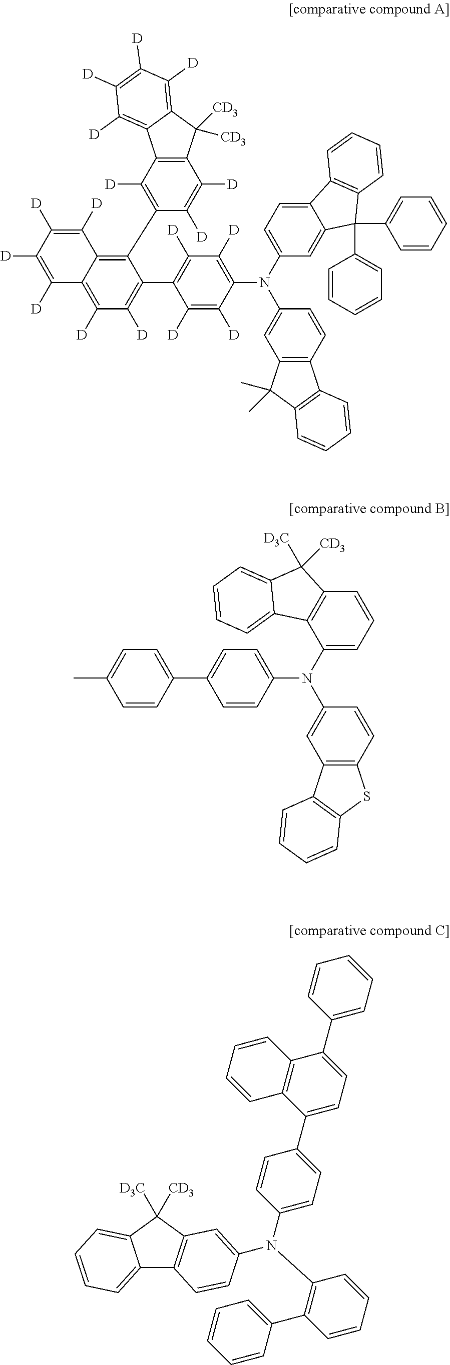 [SMILES: [2H]c1c([2H])c([2H])c2c(c1[2H])-c1c([2H])c(-c3c(-c4c([2H])c([2H])c(N(c5ccc6c(c5)C(C)(C)c5ccccc5-6)c5ccc6c(c5)C(c5ccccc5)(c5ccccc5)c5ccccc5-6)c([2H])c4[2H])c([2H])c([2H])c4c([2H])c([2H])c([2H])c([2H])c34)c([2H])c([2H])c1C2(C([2H])([2H])[2H])C([2H])([2H])[2H] ; [2H]C([2H])([2H])C1(C([2H])([2H])[2H])c2ccccc2-c2c(N(c3ccc(-c4ccc(C)cc4)cc3)c3ccc4sc5ccccc5c4c3)cccc21 ; [2H]C([2H])([2H])C1(C([2H])([2H])[2H])c2ccccc2-c2ccc(N(c3ccc(-c4ccc(-c5ccccc5)c5ccccc45)cc3)c3ccccc3-c3ccccc3)cc21]

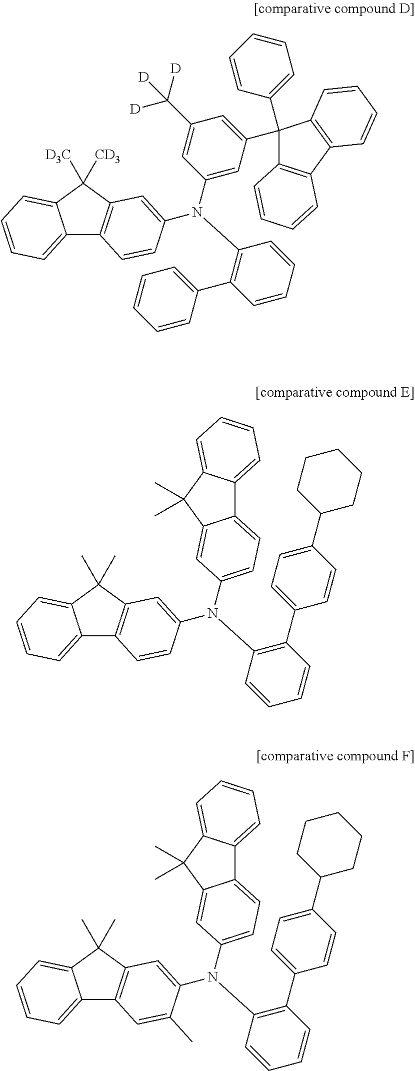 [SMILES: [2H]C([2H])([2H])c1cc(N(c2ccc3c(c2)C(C([2H])([2H])[2H])(C([2H])([2H])[2H])c2ccccc2-3)c2ccccc2-c2ccccc2)cc(C2(c3ccccc3)c3ccccc3-c3ccccc32)c1 ; CC1(C)c2ccccc2-c2ccc(N(c3ccc4c(c3)C(C)(C)c3ccccc3-4)c3ccccc3-c3ccc(C4CCCCC4)cc3)cc21 ; Cc1cc2c(cc1N(c1ccc3c(c1)C(C)(C)c1ccccc1-3)c1ccccc1-c1ccc(C3CCCCC3)cc1)C(C)(C)c1ccccc1-2]

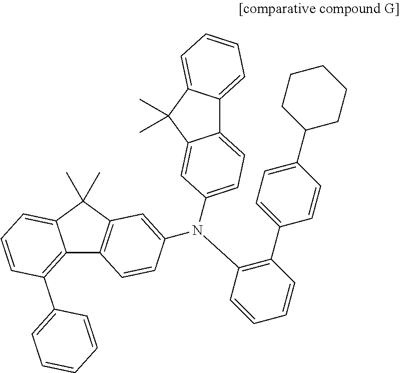 [SMILES: CC1(C)c2ccccc2-c2ccc(N(c3ccc4c(c3)C(C)(C)c3cccc(-c5ccccc5)c3-4)c3ccccc3-c3ccc(C4CCCCC4)cc3)cc21]

The electroluminescence (EL) characteristics were measured using PR-650 from Photoresearch by applying a forward bias DC voltage to the organic electroluminescence devices manufactured by Examples 1 to 35 and Comparative Examples 1 to 7 of the present invention. As a result of the measurement, the T95 lifespan was measured using a lifespan measuring device manufactured by Max Science at a standard brightness of 2,500 cd/m². Table 4 shows the results of the device fabrication and evaluation.

The measuring apparatus can evaluate the performance of new materials compared to comparative compounds under identical conditions, without being affected by possible daily fluctuations in deposition rate, vacuum quality or other parameters.

During the evaluation, one batch contains 4 identically prepared OLEDs including a comparative compound, and the performance of a total of 12 OLEDs is evaluated in 3 batches, so the value of the experimental results obtained in this way indicates statistical significance.

TABLE 4

| | | | Current | | | |
|---|---|---|---|---|---|---|
| | | Driving | Density | Brightness | Efficiency | |
| | compound | voltage | (mA/cm²) | (cd/m²) | (cd/A) | T(95) |
| comparative | comparative | 5.4 | 16.2 | 2500.0 | 15.4 | 87.7 |
| example 1 | compound A | | | | | |
| comparative | comparative | 5.3 | 15.3 | 2500.0 | 16.3 | 90.9 |
| example 2 | compound B | | | | | |
| comparative | comparative | 5.2 | 13.7 | 2500.0 | 18.2 | 98.6 |
| example 3 | compound C | | | | | |
| comparative | comparative | 5.3 | 14.3 | 2500.0 | 17.5 | 94.8 |
| example 4 | compound D | | | | | |
| comparative | comparative | 5.3 | 15.7 | 2500.0 | 15.9 | 95.3 |
| example 5 | compound E | | | | | |
| comparative | comparative | 5.4 | 13.5 | 2500.0 | 18.5 | 96.5 |
| example 6 | compound F | | | | | |
| comparative | comparative | 5.4 | 14.0 | 2500.0 | 17.8 | 94.2 |
| example 7 | compound G | | | | | |
| example 1 | P-1 | 4.3 | 7.2 | 2500.0 | 34.7 | 121.2 |
| example 2 | P-12 | 4.2 | 7.0 | 2500.0 | 35.9 | 120.9 |
| example 3 | P-15 | 4.3 | 7.4 | 2500.0 | 33.8 | 120.0 |
| example 4 | P-25 | 4.2 | 7.1 | 2500.0 | 35.0 | 121.2 |
| example 5 | P-36 | 4.3 | 7.2 | 2500.0 | 34.9 | 123.7 |
| example 6 | P-40 | 4.3 | 7.3 | 2500.0 | 34.3 | 119.7 |
| example 7 | P-47 | 4.3 | 7.9 | 2500.0 | 31.7 | 117.4 |
| example 8 | P-60 | 4.4 | 7.6 | 2500.0 | 32.8 | 115.8 |
| example 9 | P-64 | 4.4 | 7.7 | 2500.0 | 32.6 | 111.1 |
| example 10 | P-68 | 4.4 | 7.8 | 2500.0 | 32.1 | 114.1 |
| example 11 | P-75 | 4.4 | 8.3 | 2500.0 | 30.2 | 110.6 |
| example 12 | P-80 | 4.4 | 7.5 | 2500.0 | 33.5 | 116.8 |
| example 13 | P-81 | 4.1 | 6.7 | 2500.0 | 37.3 | 124.8 |
| example 14 | P-84 | 4.1 | 6.8 | 2500.0 | 36.7 | 127.2 |
| example 15 | P-85 | 4.2 | 6.9 | 2500.0 | 36.3 | 124.5 |
| example 16 | P-89 | 4.1 | 6.3 | 2500.0 | 40.0 | 129.7 |
| example 17 | P-92 | 4.1 | 6.9 | 2500.0 | 36.1 | 124.9 |
| example 18 | P-102 | 4.3 | 6.7 | 2500.0 | 37.1 | 125.3 |
| example 19 | P-114 | 4.2 | 6.6 | 2500.0 | 38.0 | 124.6 |
| example 20 | P-117 | 4.2 | 6.5 | 2500.0 | 38.6 | 124.7 |
| example 21 | P-119 | 4.2 | 6.4 | 2500.0 | 39.2 | 125.5 |
| example 22 | P-120 | 4.2 | 6.9 | 2500.0 | 36.1 | 128.5 |
| example 23 | P-128 | 4.4 | 8.2 | 2500.0 | 30.5 | 110.9 |
| example 24 | P-131 | 4.3 | 8.0 | 2500.0 | 31.2 | 111.4 |
| example 25 | P-137 | 4.3 | 7.8 | 2500.0 | 32.0 | 118.6 |
| example 26 | P-138 | 4.4 | 8.2 | 2500.0 | 30.6 | 116.9 |
| example 27 | P-141 | 4.1 | 6.3 | 2500.0 | 39.9 | 130.1 |
| example 28 | P-142 | 4.2 | 6.7 | 2500.0 | 37.5 | 125.8 |
| example 29 | P-143 | 4.8 | 8.5 | 2500.0 | 29.3 | 109.6 |
| example 30 | P-144 | 4.3 | 6.9 | 2500.0 | 36.3 | 124.2 |
| example 31 | P-148 | 4.7 | 8.4 | 2500.0 | 29.9 | 108.7 |
| example 32 | P-152 | 4.2 | 7.0 | 2500.0 | 35.6 | 120.4 |
| example 33 | P-154 | 4.5 | 8.1 | 2500.0 | 30.9 | 111.5 |
| example 34 | P-158 | 4.4 | 7.7 | 2500.0 | 32.4 | 114.9 |
| example 35 | P-160 | 4.2 | 6.5 | 2500.0 | 38.4 | 126.3 |

Comparative compounds A to D are similar to the compound of the present invention in that they are tertiary amine compounds comprising a structure in which CDs is disubstituted at position 9 of fluorene (hereinafter, parent nucleus), but the compound of the present invention is different from the comparative compounds in that it necessarily comprises a substituent comprising an o-biphenyl skeleton substituted with an alkyl group or an aliphatic ring group. Comparative compounds E to G comprise a substituent comprising an o-biphenyl skeleton substituted with an alkyl group or an aliphatic ring group, and are similar to the compounds of the present invention in that fluorene is disubstituted, but differ from the compounds of the present invention in that CD3 is not substituted at position 9 of fluorene.

In the case of comparative compound A, it is similar to the compound of the present invention in that one of the substituents of the remaining amine other than the parent nucleus contains an alkyl group (9, 9′-dimethylfluorene), but the compound of the present invention is different from comparative compound A in that an alkyl group is substituted at the terminal of the substituent. In order to confirm the energy level difference of the compound due to such structural difference, data measured using the DFT method (B3LYP/6-31g(D)) of the Gaussian program for the comparative compound A and the compound P-80 of the present invention having the most similar structure thereto are as shown in Table 5.

TABLE 5

| compound | Comparative compound A | P-80 |
|---|---|---|
| HOMO(eV) | −4.701 | −4.769 |

As can be seen from the results in Table 5, it can be confirmed that the HOMO Energy Level (hereinafter, HOMO) value of compound P-80 of the present invention is deeper than that of comparative compound A. As a result, when the compound of the present invention is applied to a device, it is thought that hole transfer from the emitting auxiliary layer to the emitting layer becomes easier than with the comparative compound A, so that the hole accumulation in the device decreases and the charge balance increases, thereby significantly improving the efficiency and lifespan of the device.

Next, in the case of comparative compounds B to D, they are similar to the compounds of the present invention in that one of the substituents of the remaining amine other than the parent nucleus includes a biphenyl moiety, but comparative compound B differs from the compound of the present invention in that it contains p-biphenyl rather than o-biphenyl, and comparative compound c and comparative compound D differ from the compound of the present invention in that they contain an o-biphenyl skeleton, but the alkyl or aliphatic ring group is unsubstituted. In order to confirm the influence due to the difference in these substituents, the data for measuring the Reorganization Energy (unit: eV, hereinafter REₕₒₗₑ) of compounds of the present invention similar to Compounds B to D using molecular simulation (Schrodinger Materials Science Suite 4.9.128) are as shown in Table 6.

TABLE 6

| compound | REₕₒₗₑ(eV) |
|---|---|
| comparative compound B | 0.1900 |
| comparative compound C | 0.1603 |
| comparative compound D | 0.1636 |
| P-40 | 0.1500 |
| P-119 | 0.1546 |
| P-120 | 0.1597 |

As can be seen from the results in Table 6, it can be confirmed that the REₕₒₗₑ values of the compounds of the present invention are significantly lower than the REₕₒₗₑ values of comparative compounds B to D. As a result, when the compound of the present invention is applied to a device, hole hopping occurs more quickly within the material than in comparative compounds, thereby improving hole mobility, allowing holes to be transferred to the emitting layer more quickly, reducing electron quenching in the emitting layer, and improving charge balance in the emitting layer as exciton formation becomes easier, resulting in significant improvements in the lifespan, operation, and efficiency of the device.

In the case of comparative compounds E to G, the presence or absence of the substitution of CD₃ at position 9 of fluorene is different from that of the compound of the present invention. In the case of the compound of the present invention, it is judged that the compound exhibited superior performance in the device compared to the comparative compounds because the structural stability increased as CD₃ was substituted at position 9, which is relatively weak in bonding in the fluorenyl group.

From another aspect, based on the device measurement results, in the case where Ar¹ is substituted with fluorene in the compound of the present invention, the device performance was measured to be better when an additional substituent was further substituted on the fluorene skeleton, and in particular, compounds having an additional substituent on the ring bonded to the amine in the fluorene skeleton showed better device results than other cases.

In other words, as can be seen from the results in Tables 4 to 6, it can be confirmed that the compound satisfying both the structural features and the configuration disclosed in the present invention exhibits a remarkable effect in the organic electronic element, compared to the comparative compounds A to G having a structurally similar configuration to the compound of the present invention. This means that the compound of the present invention satisfying all the specific configurations exhibits a remarkable effect, compared to other comparative compounds not described in the present specification.

These results suggest that even for compounds with similar molecular components, the properties of the compound, such as hole characteristics, luminous efficiency characteristics, energy levels, hole injection and mobility characteristics, charge balance of holes and electrons, volume density, and intermolecular distance, can differ significantly to an extent that is difficult to predict depending on the type and position of the substituted substituent, and that the performance of an element may vary due to complex factors, rather than a single configuration affecting the outcome of the entire device.

In the case of an emitting auxiliary layer, the relationship between the hole transport layer and the emitting layer (host) must be understood. Therefore, even if a similar core is used, it would be very difficult for even a person skilled in the art to infer the characteristics exhibited in the emitting auxiliary layer in which the compound of the present invention is used.

Additionally, the evaluation results of the described device fabrication explained the device characteristics in which the compound of the present invention was applied only to the emitting auxiliary layer, but the compound of the present invention can be used by applying it to the hole transport layer or by applying it to both the hole transport layer and the emitting auxiliary layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

What is claimed is:

1. A compound represented by Formula 1: 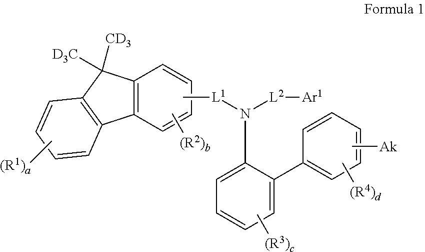
wherein:
R¹, R², R³ and R⁴ are independently the same or different from each other and are independently selected from the group consisting of hydrogen; deuterium; an C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and a C₃-C₃₀ aliphatic ring, or a plurality of adjacent groups may be bonded to each other to form a ring,
L¹ and L² are independently selected from the group consisting of a single bond; a C₆-C₆₀ arylene group; a fluorenylene group; and a C₂-C₆₀ heterocyclic group including at least one heteroatom of O, N, S, Si or P;
Ar¹ is an C₆-C₆₀ aryl group; a fluorenyl group; a C₂-C₆₀ heterocyclic group including at least one heteroatom of O, N, S, Si or P;
Ak is a C₁-C₅₀ alkyl group; or a C₃-C₃₀ aliphatic ring group;
a, c and d are independently an integer of 0 to 4, b is an integer of 0 to 3,
wherein the aryl group, arylene group, heterocyclic group, fluorenyl group, fluorenylene group, aliphatic ring group, fused ring group, alkyl group, alkenyl group, alkynyl group, alkoxyl group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; a C₃-C₂₀ aliphatic ring; a C₇-C₂₀ arylalkyl group; a C₅-C₂₀ arylalkenyl group; and a C₇-C₂₀ alkylaryl group; and also the hydrogen of these substituents may be further substituted with one or more deuterium, and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound according to claim 1, wherein Formula 1 is represented by any of Formulas 1-1 to 1-4: 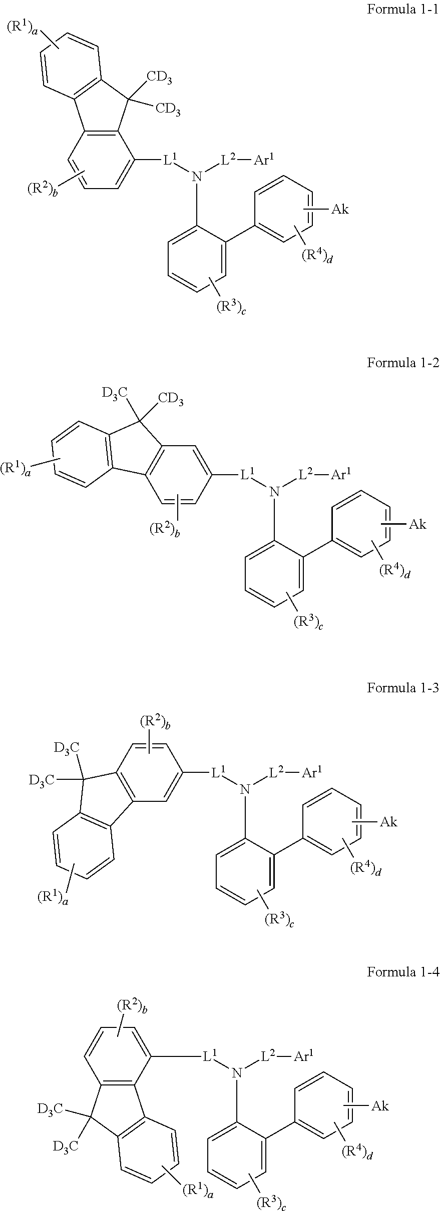
wherein, R¹, R², R³, R⁴, a, b, c, d, L¹, L², Ar¹ and Ak are the same as defined in Formula 1.

3. The compound according to claim 1, wherein Ak is represented by any of Formulas Ak-1 to Ak-8: 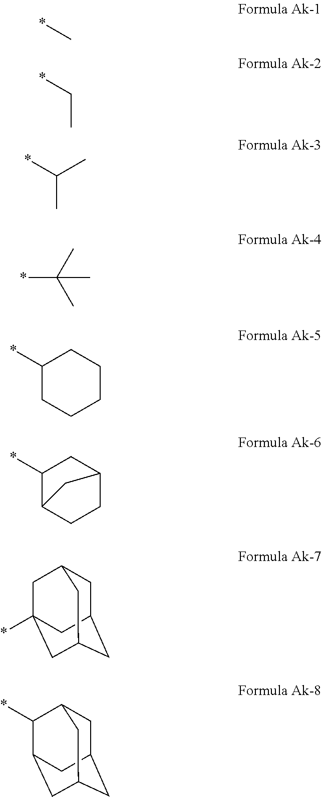
wherein:
* indicates a position to be bonded, and each of Formulas Ak-1 to Ak-8 may be further substituted with one or more deuterium.

4. The compound according to claim 1, wherein Ar¹ is represented by any of Formulas Ar-1 to Ar-11: 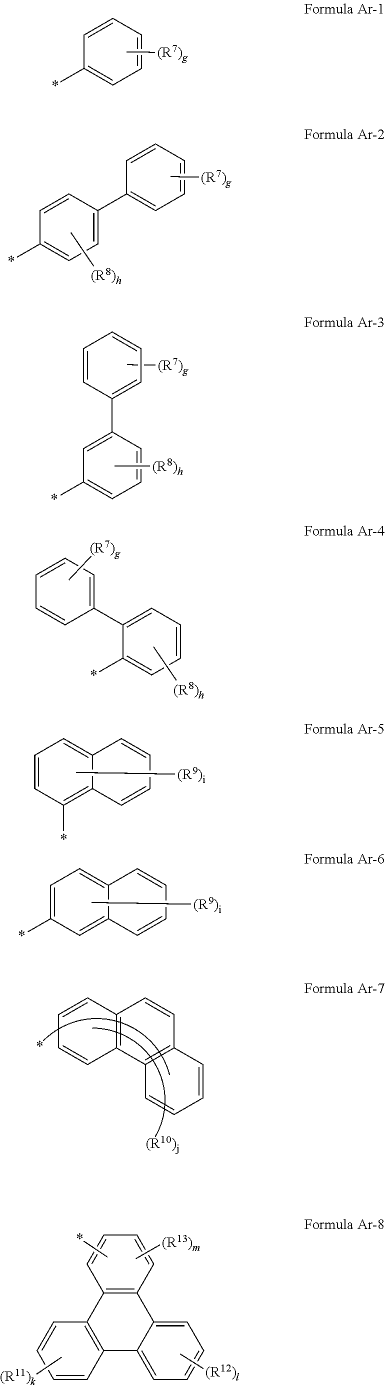 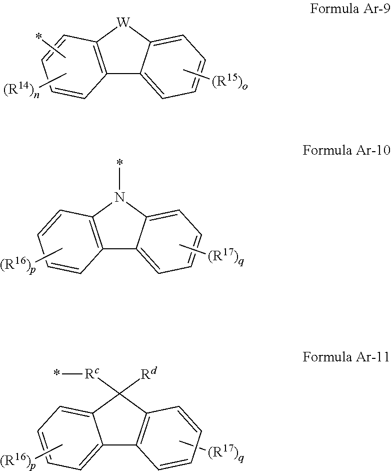
wherein:
R⁷, R⁸, R⁹, R¹⁰, R¹¹, R¹², R¹³, R¹⁴, R¹⁵, R¹⁶ and R¹⁷ are the same or different from each other, and are independently selected from the group consisting of hydrogen; or deuterium; a halogen; a silane group; a siloxane group; a boron group; a germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic group; a C₇-C₂₀ arylalkyl group; a C₈-C₂₀ arylalkenyl group; and a C₇-C₂₀ alkylaryl group, or a plurality of adjacent groups may be bonded to each other to form a ring,
R^{c} is selected from the group consisting of a C₁-C₆₀ alkyl group; an C₆-C₆₀ aryl group; and a C₂-C₆₀ heterocyclic group including at least one heteroatom of O, N, S, Si or P;
R^{d} is selected from the group consisting of hydrogen; deuterium; a C₁-C₅₀ alkyl group; an C₆-C₆₀ aryl group; and a C₂-C₆₀ heterocyclic group including at least one heteroatom of O, N, S, Si or P;
R^{c} and R^{d} can be bonded to each other to form a spiro ring,
g is an integer of 0 to 5; h, k, l, o, p and q are independently an integer of 0 to 4; i is an integer from 0 to 7; j is an integer from 0 to 9; m and n are independently an integer of 0 to 3,
W is O, S, C(R²⁸)(R²⁹) or NR³⁰,
R²⁸, R²⁹ and R³⁰ are selected from the group consisting of a C₁-C₂₀ alkyl group; a C₂-C₂₀ alkenyl group; C₆-C₂₀ aryl group; C₆-C₂₀ aryl group substituted with deuterium; a fluorenyl group; a C₂-C₂₀ heterocyclic group including at least one heteroatom of O, N, S, Si or P; a C₃-C₂₀ aliphatic group; a C₇-C₂₀ arylalkyl group; a C₈-C₂₀ arylalkenyl group; and a C₇-C₂₀ alkylaryl group, or R²⁷ and R²⁸ may be bonded to each other to form a ring,
* indicates a position to be bonded.

5. The compound according to claim 1, wherein L¹ and L² are selected from a single bond or one of Formulas L-1 to L-27: 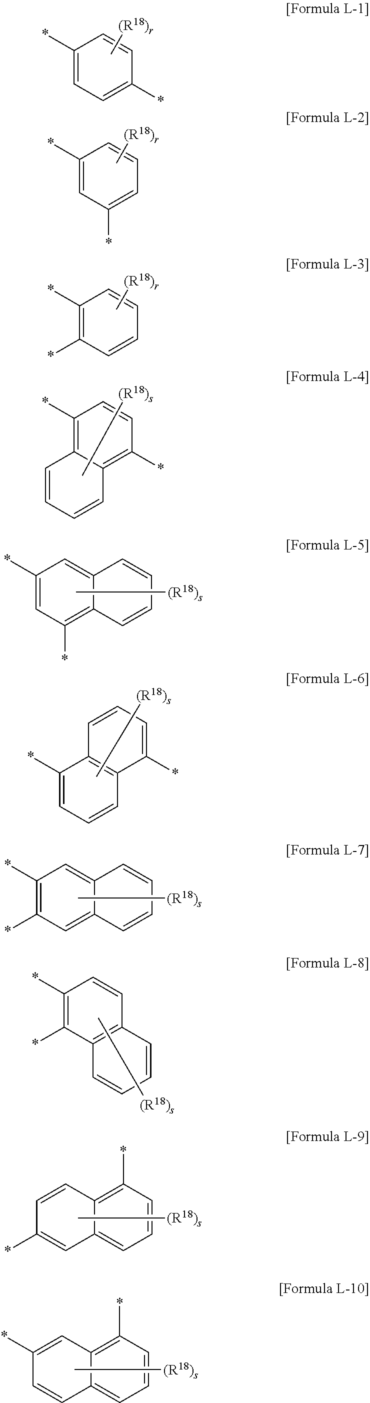 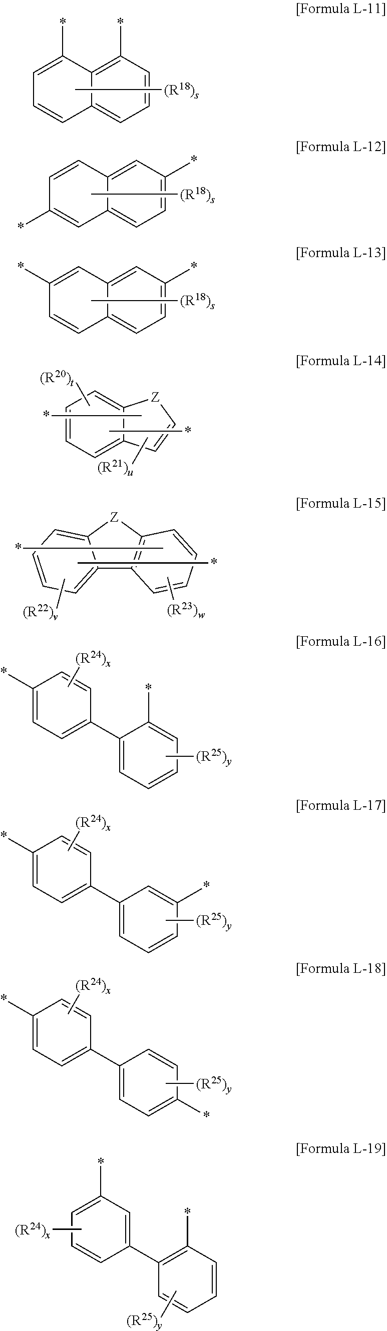 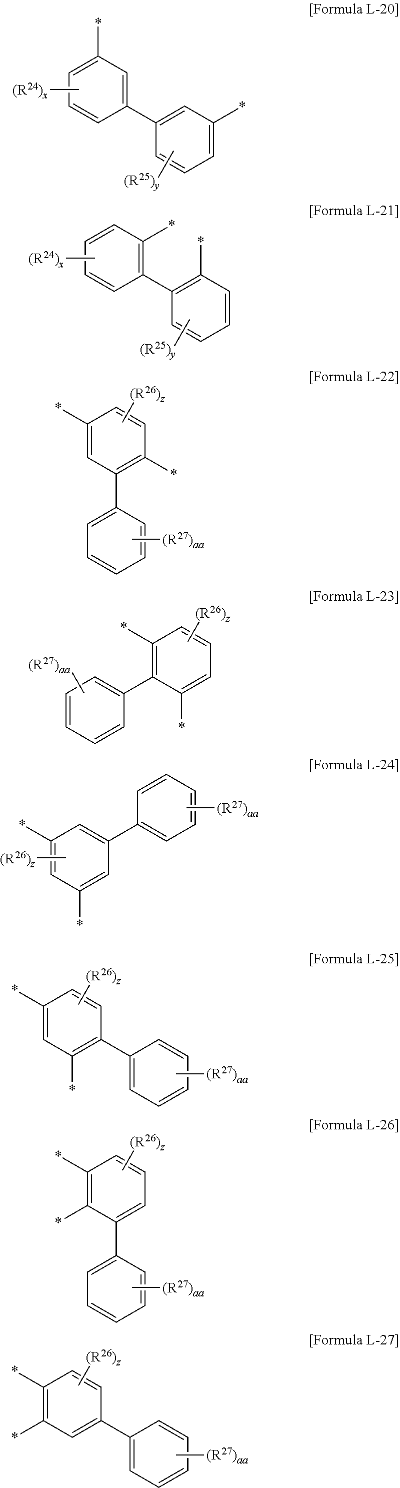
wherein:
Z is O, S, C(R³¹)(R³²) or N—Ar⁵,
R¹⁸, R¹⁹, R²⁰, R²¹, R²², R²³, R²⁴, R²⁵, R²⁶ and R²⁷ are independently selected from the group consisting of hydrogen; or deuterium; a halogen; a silane group; a siloxane group; a boron group; a germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic group; a C₇-C₂₀ arylalkyl group; a C₈-C₂₀ arylalkenyl group; and a C₇-C₂₀ alkylaryl group, or a plurality of adjacent groups may be bonded to each other to form a ring,
R³¹ and R³² are selected from the group consisting of a C₁-C₂₀ alkyl group; a C₂-C₂₀ alkenyl group; C₆-C₂₀ aryl group; C₆-C₂₀ aryl group substituted with deuterium; a fluorenyl group; a C₂-C₂₀ heterocyclic group including at least one heteroatom of O, N, S, Si or P; a C₃-C₂₀ aliphatic group; a C₇-C₂₀ arylalkyl group; a C₈-C₂₀ arylalkenyl group; and a C₇-C₂₀ alkylaryl group, or R²⁷ and R²⁸ may be bonded to each other to form a ring,
Ar⁵ is selected from the group consisting of a C₁-C₂₀ alkyl group; a C₆-C₂₀ aryl group; a C₆-C₂₀ aryl group substituted with deuterium; a fluorenyl group; and a C₂-C₂₀ heterocyclic group including at least one heteroatom of O, N, S, Si or P;
r, t, v, w, x, y and z are independently an integer of 0 to 4, s is an integer of 0 to 6, u is an integer of 0 to 2, aa is an integer of 0 to 5; and
* indicates a position to be bonded.

6. The compound according to claim 1, wherein Ar¹ is represented by Formula Ar-12 or Formula Ar-13: 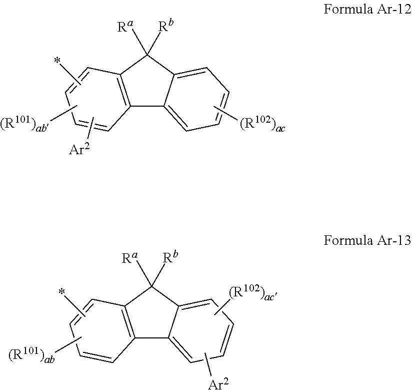
wherein:
R^{a} and R^{b} are independently selected from the group consisting of a C₁-C₂₀ alkyl group; a C₂-C₂₀ alkenyl group; a C₆-C₂₀ aryl group; a C₆-C₂₀ aryl group substituted with deuterium; a fluorenyl group; a C₂-C₂₀ heterocyclic group including at least one heteroatom of O, N, S, Si or P; a C₃-C₂₀ aliphatic group; a C₇-C₂₀ arylalkyl group; a C₈-C₂₀ arylalkenyl group; and a C₇-C₂₀ alkylaryl group, or R^{a} and R^{b} can be bonded to each other to form a spiro ring,
R¹⁰¹ and R¹⁰² are independently selected from the group consisting of hydrogen; or deuterium; a halogen; a silane group; a siloxane group; a boron group; a germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic group; a C₇-C₂₀ arylalkyl group; a C₈-C₂₀ arylalkenyl group; and a C₇-C₂₀ alkylaryl group, or a plurality of adjacent groups may be bonded to each other to form a ring,
Ar² is selected from the group consisting of a C₁-C₂₀ alkyl group substituted or unsubstituted with deuterium; a C₆-C₂₀ aryl group substituted or unsubstituted with deuterium; a C₂-C₂₀ heterocyclic group including at least one heteroatom of O, N, S, Si or P; and a C₇-C₂₀ alkylaryl group;
ab′ is an integer of 0 to 2, ac is an integer of 0 to 4, and ab and ac′ are independently an integer of 0 to 3; and
* indicates a position to be bonded.

7. The compound according to claim 1, wherein the compound represented by Formula 1 is any of compounds P-1 to P-160:

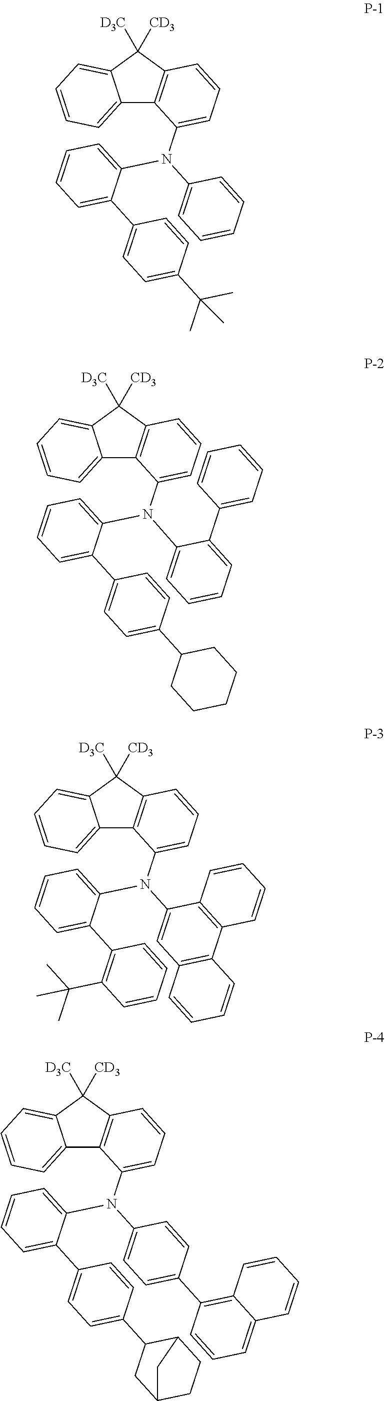 [SMILES: [2H]C([2H])([2H])C1(C([2H])([2H])[2H])c2ccccc2-c2c(N(c3ccccc3)c3ccccc3-c3ccc(C(C)(C)C)cc3)cccc21 ; [2H]C([2H])([2H])C1(C([2H])([2H])[2H])c2ccccc2-c2c(N(c3ccccc3-c3ccccc3)c3ccccc3-c3ccc(C4CCCCC4)cc3)cccc21 ; [2H]C([2H])([2H])C1(C([2H])([2H])[2H])c2ccccc2-c2c(N(c3ccccc3-c3ccccc3C(C)(C)C)c3cc4ccccc4c4ccccc34)cccc21 ; [2H]C([2H])([2H])C1(C([2H])([2H])[2H])c2ccccc2-c2c(N(c3ccc(-c4cccc5ccccc45)cc3)c3ccccc3-c3ccc(C4CC5CCC4C5)cc3)cccc21]

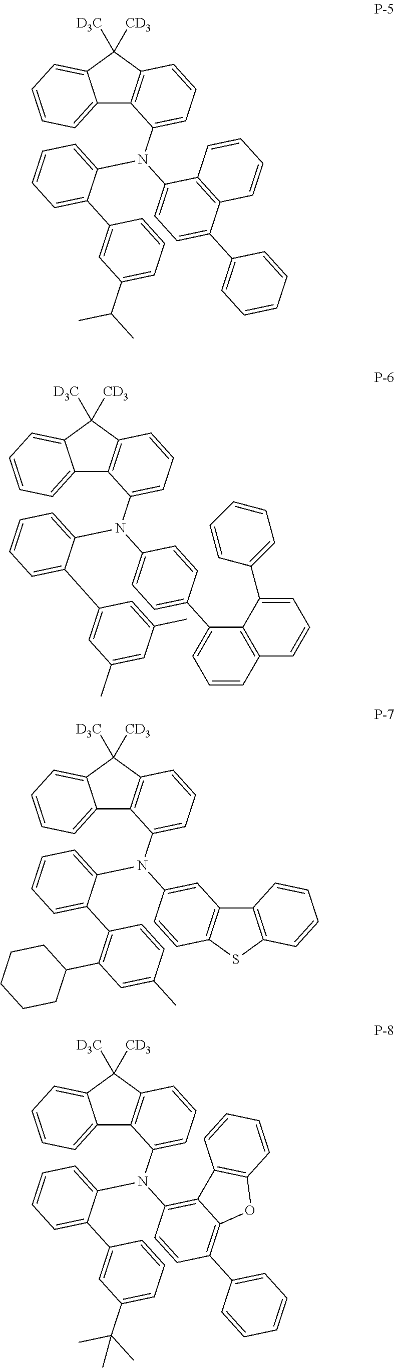

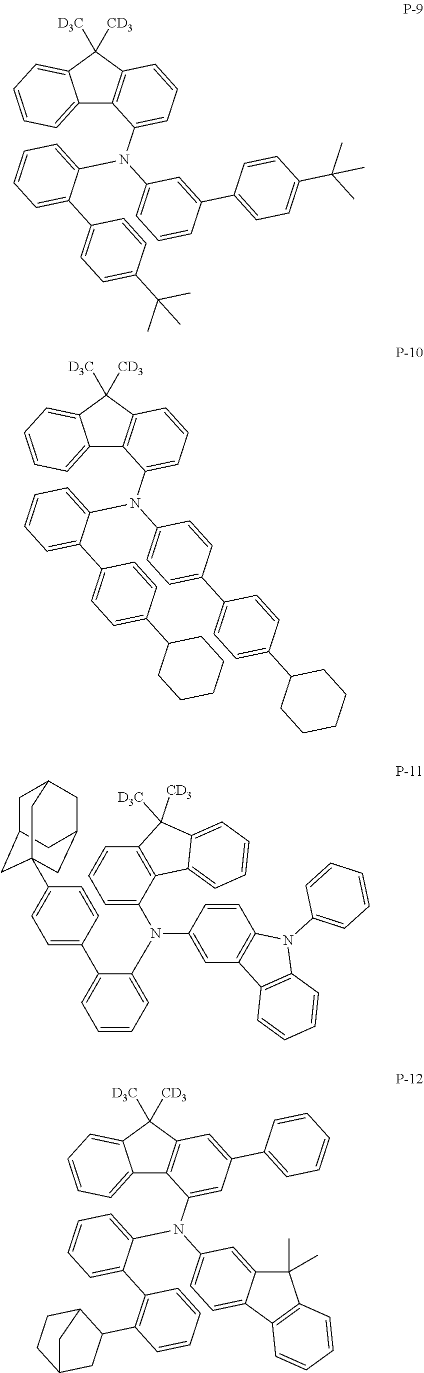

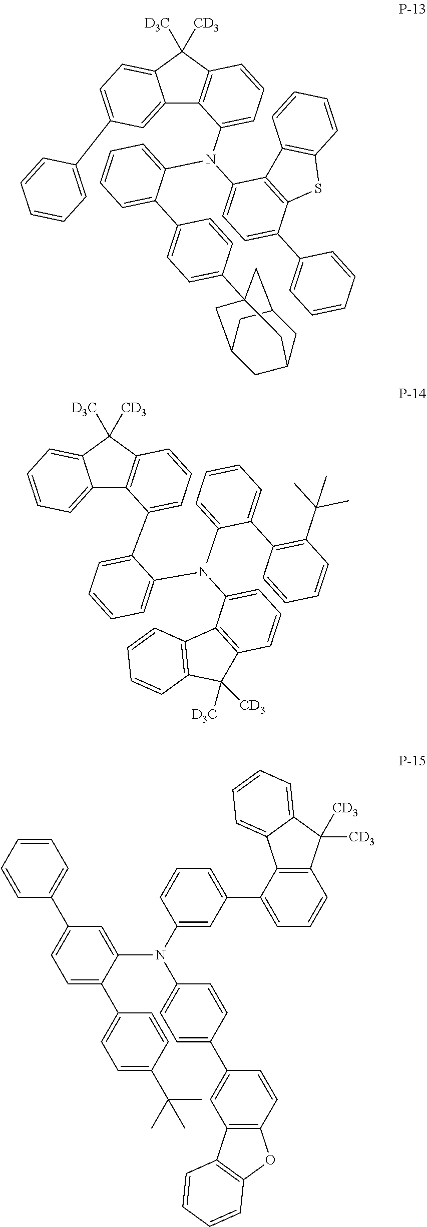 [SMILES: [2H]C([2H])([2H])C1(C([2H])([2H])[2H])c2ccc(-c3ccccc3)cc2-c2c(N(c3ccccc3-c3ccc(C45CC6CC(CC(C6)C4)C5)cc3)c3ccc(-c4ccccc4)c4sc5ccccc5c34)cccc21 ; [2H]C([2H])([2H])C1(C([2H])([2H])[2H])c2ccccc2-c2c(-c3ccccc3N(c3ccccc3-c3ccccc3C(C)(C)C)c3cccc4c3-c3ccccc3C4(C([2H])([2H])[2H])C([2H])([2H])[2H])cccc21 ; [2H]C([2H])([2H])C1(C([2H])([2H])[2H])c2ccccc2-c2c(-c3cccc(N(c4ccc(-c5ccc6oc7ccccc7c6c5)cc4)c4cc(-c5ccccc5)ccc4-c4ccc(C(C)(C)C)cc4)c3)cccc21]

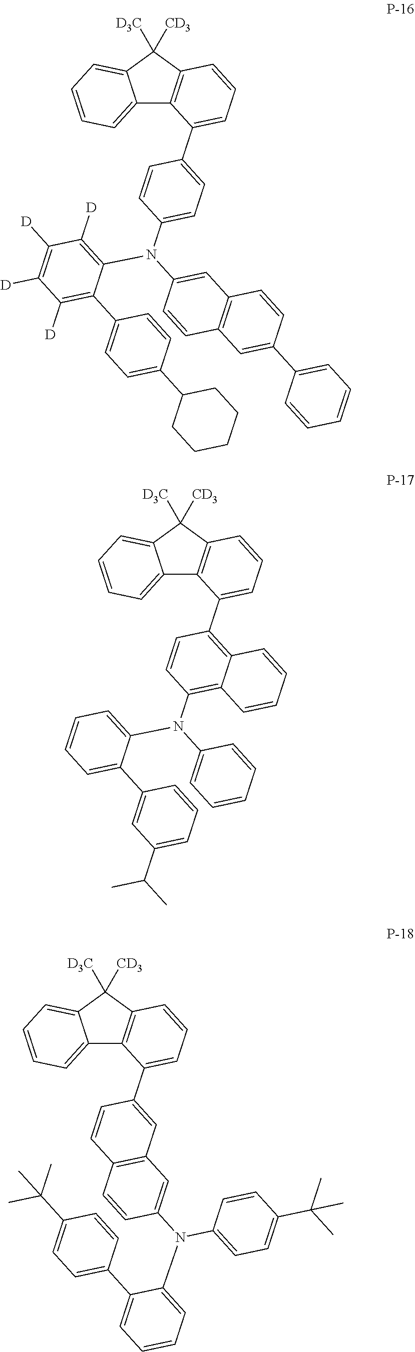

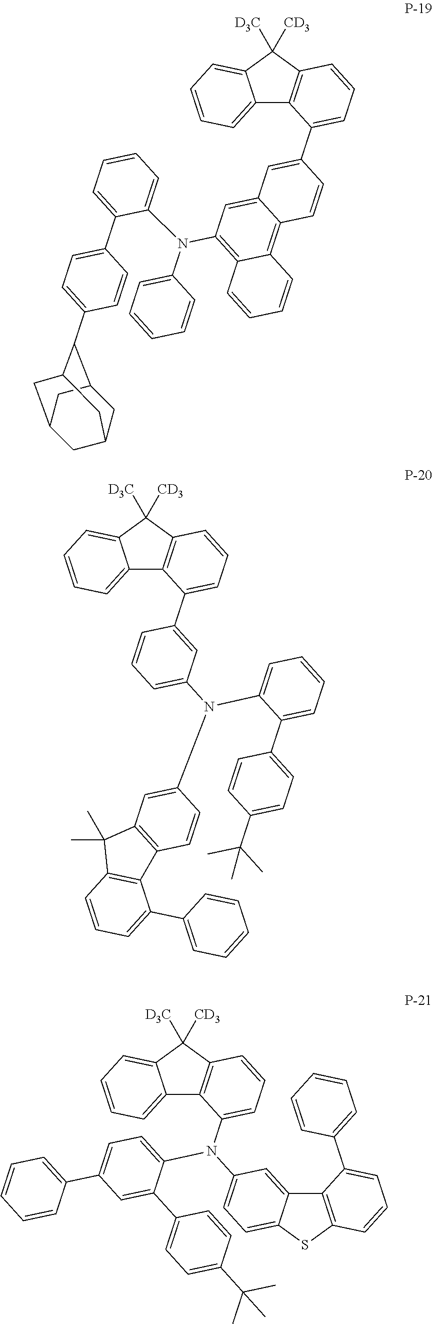 [SMILES: [2H]C([2H])([2H])C1(C([2H])([2H])[2H])c2ccccc2-c2c(-c3ccc4c(c3)cc(N(c3ccccc3)c3ccccc3-c3ccc(C5C6CC7CC(C6)CC5C7)cc3)c3ccccc34)cccc21 ; [2H]C([2H])([2H])C1(C([2H])([2H])[2H])c2ccccc2-c2c(-c3cccc(N(c4ccc5c(c4)C(C)(C)c4cccc(-c6ccccc6)c4-5)c4ccccc4-c4ccc(C(C)(C)C)cc4)c3)cccc21 ; [2H]C([2H])([2H])C1(C([2H])([2H])[2H])c2ccccc2-c2c(N(c3ccc4sc5cccc(-c6ccccc6)c5c4c3)c3ccc(-c4ccccc4)cc3-c3ccc(C(C)(C)C)cc3)cccc21]

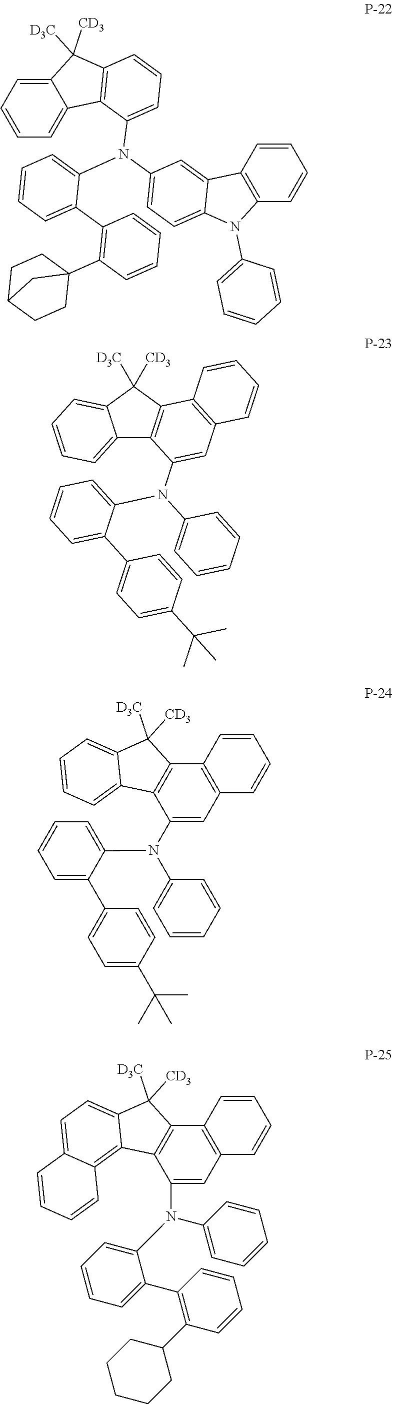

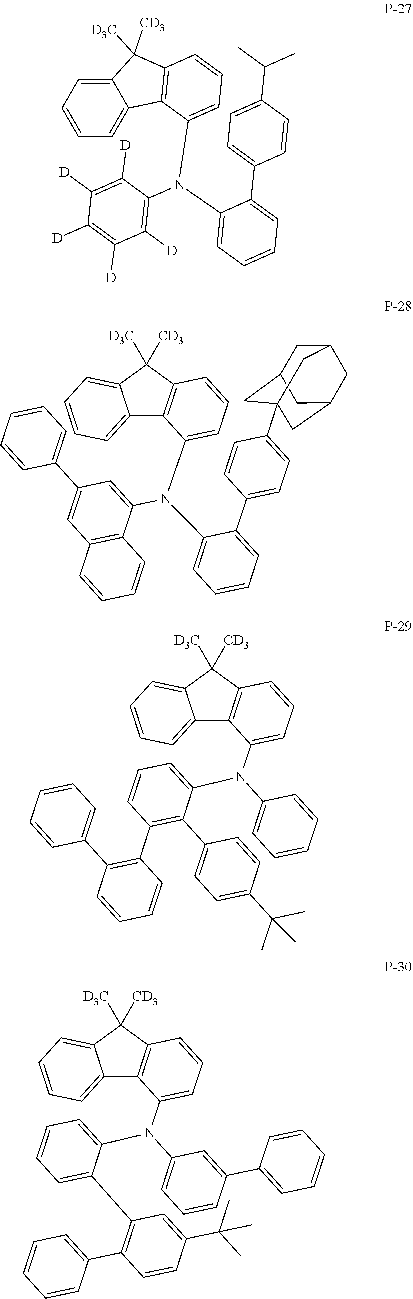 [SMILES: [2H]c1c([2H])c([2H])c(N(c2ccccc2-c2ccc(C(C)C)cc2)c2cccc3c2-c2ccccc2C3(C([2H])([2H])[2H])C([2H])([2H])[2H])c([2H])c1[2H] ; [2H]C([2H])([2H])C1(C([2H])([2H])[2H])c2ccccc2-c2c(N(c3ccccc3-c3ccc(C45CC6CC(CC(C6)C4)C5)cc3)c3cc(-c4ccccc4)cc4ccccc34)cccc21 ; [2H]C([2H])([2H])C1(C([2H])([2H])[2H])c2ccccc2-c2c(N(c3ccccc3)c3cccc(-c4ccccc4-c4ccccc4)c3-c3ccc(C(C)(C)C)cc3)cccc21 ; [2H]C([2H])([2H])C1(C([2H])([2H])[2H])c2ccccc2-c2c(N(c3cccc(-c4ccccc4)c3)c3ccccc3-c3cc(C(C)(C)C)ccc3-c3ccccc3)cccc21]

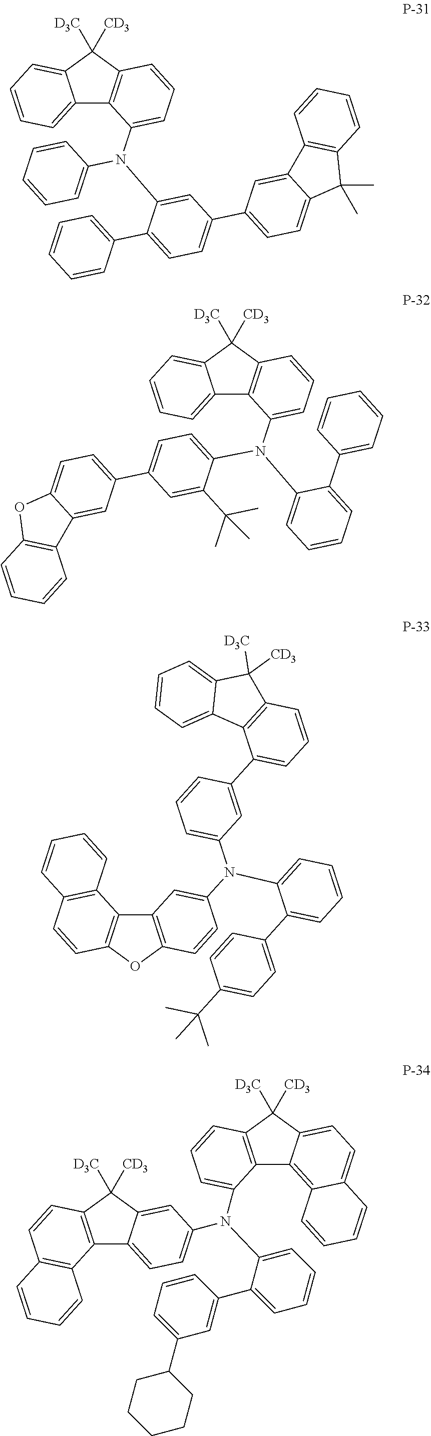

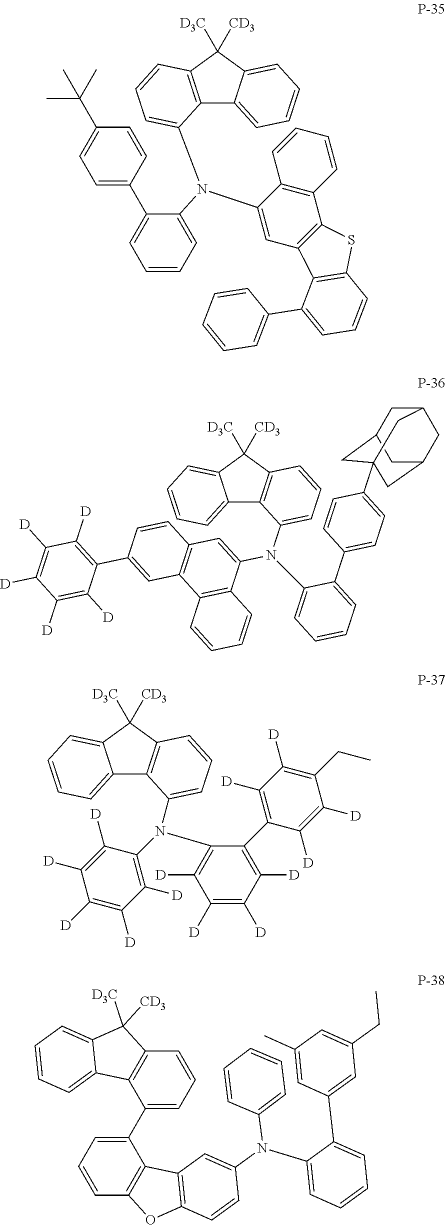

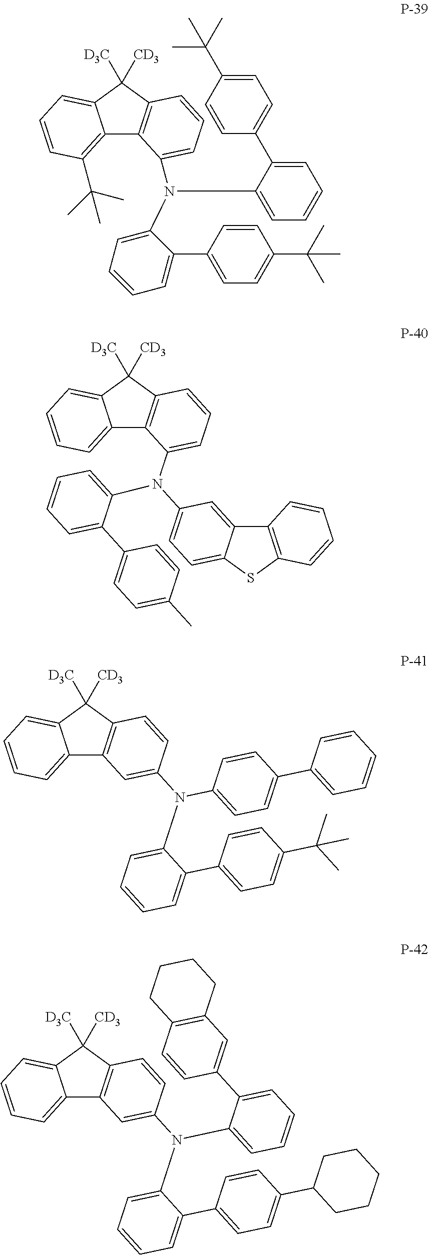 [SMILES: [2H]C([2H])([2H])C1(C([2H])([2H])[2H])c2cccc(N(c3ccccc3-c3ccc(C(C)(C)C)cc3)c3ccccc3-c3ccc(C(C)(C)C)cc3)c2-c2c(C(C)(C)C)cccc21 ; [2H]C([2H])([2H])C1(C([2H])([2H])[2H])c2ccccc2-c2c(N(c3ccc4sc5ccccc5c4c3)c3ccccc3-c3ccc(C)cc3)cccc21 ; [2H]C([2H])([2H])C1(C([2H])([2H])[2H])c2ccccc2-c2cc(N(c3ccc(-c4ccccc4)cc3)c3ccccc3-c3ccc(C(C)(C)C)cc3)ccc21 ; [2H]C([2H])([2H])C1(C([2H])([2H])[2H])c2ccccc2-c2cc(N(c3ccccc3-c3ccc(C4CCCCC4)cc3)c3ccccc3-c3ccc4c(c3)CCCC4)ccc21]

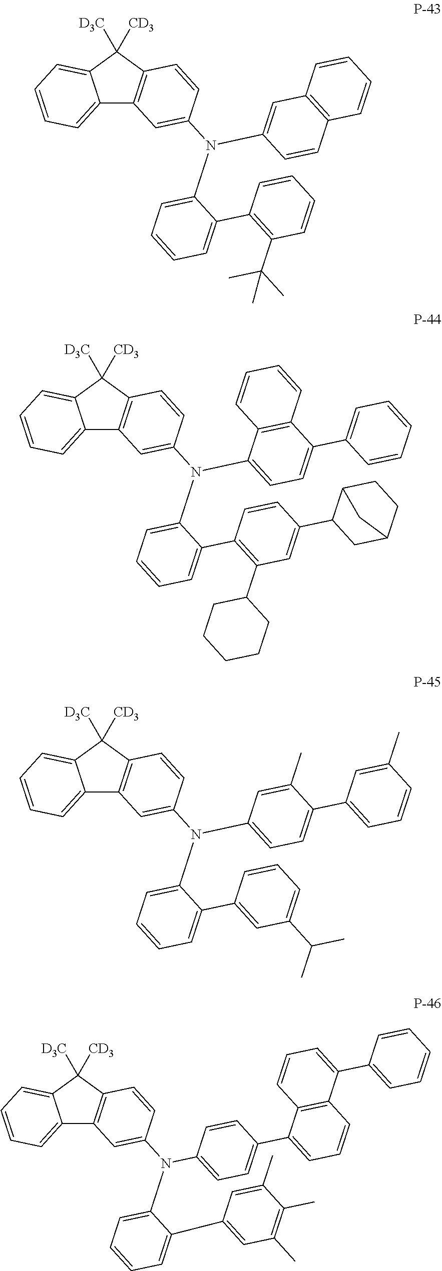 [SMILES: [2H]C([2H])([2H])C1(C([2H])([2H])[2H])c2ccccc2-c2cc(N(c3ccc4ccccc4c3)c3ccccc3-c3ccccc3C(C)(C)C)ccc21 ; [2H]C([2H])([2H])C1(C([2H])([2H])[2H])c2ccccc2-c2cc(N(c3ccccc3-c3ccc(C4CC5CCC4C5)cc3C3CCCCC3)c3ccc(-c4ccccc4)c4ccccc34)ccc21 ; [2H]C([2H])([2H])C1(C([2H])([2H])[2H])c2ccccc2-c2cc(N(c3ccc(-c4cccc(C)c4)c(C)c3)c3ccccc3-c3cccc(C(C)C)c3)ccc21 ; [2H]C([2H])([2H])C1(C([2H])([2H])[2H])c2ccccc2-c2cc(N(c3ccc(-c4cccc5c(-c6ccccc6)cccc45)cc3)c3ccccc3-c3cc(C)c(C)c(C)c3)ccc21]

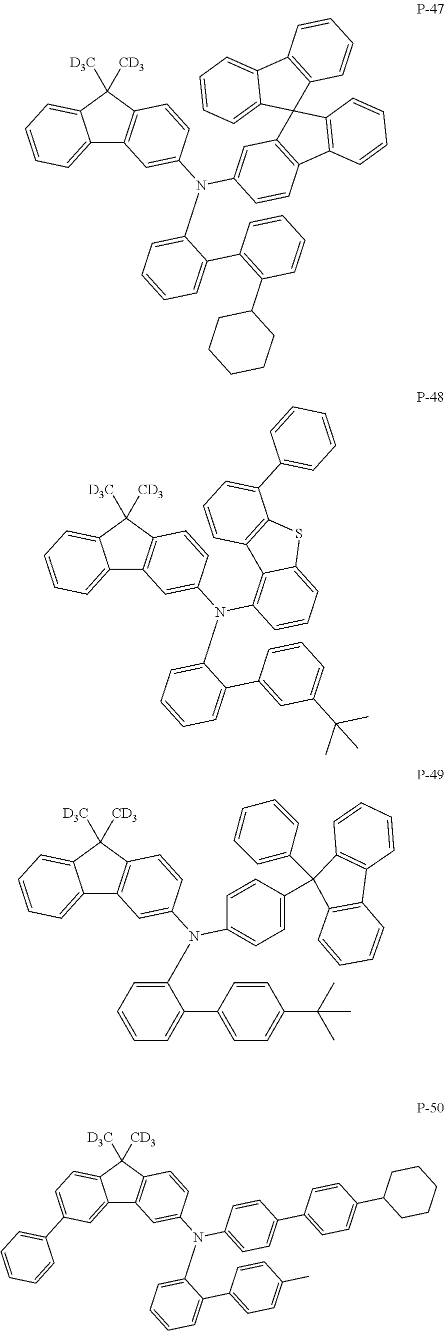

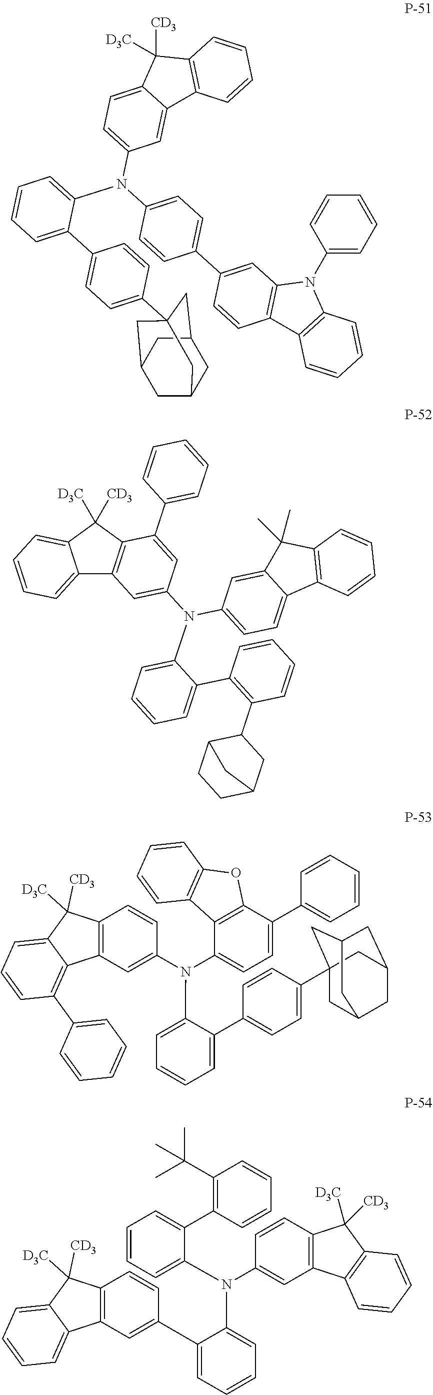

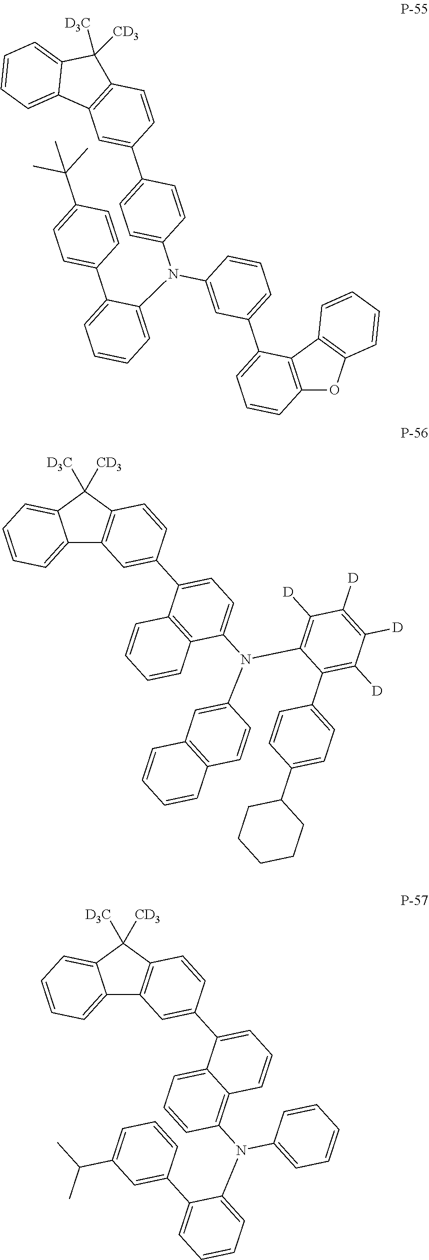 [SMILES: [2H]C([2H])([2H])C1(C([2H])([2H])[2H])c2ccccc2-c2cc(-c3ccc(N(c4cccc(-c5cccc6oc7ccccc7c56)c4)c4ccccc4-c4ccc(C(C)(C)C)cc4)cc3)ccc21 ; [2H]c1c([2H])c([2H])c(N(c2ccc3ccccc3c2)c2ccc(-c3ccc4c(c3)-c3ccccc3C4(C([2H])([2H])[2H])C([2H])([2H])[2H])c3ccccc23)c(-c2ccc(C3CCCCC3)cc2)c1[2H] ; [2H]C([2H])([2H])C1(C([2H])([2H])[2H])c2ccccc2-c2cc(-c3cccc4c(N(c5ccccc5)c5ccccc5-c5cccc(C(C)C)c5)cccc34)ccc21]

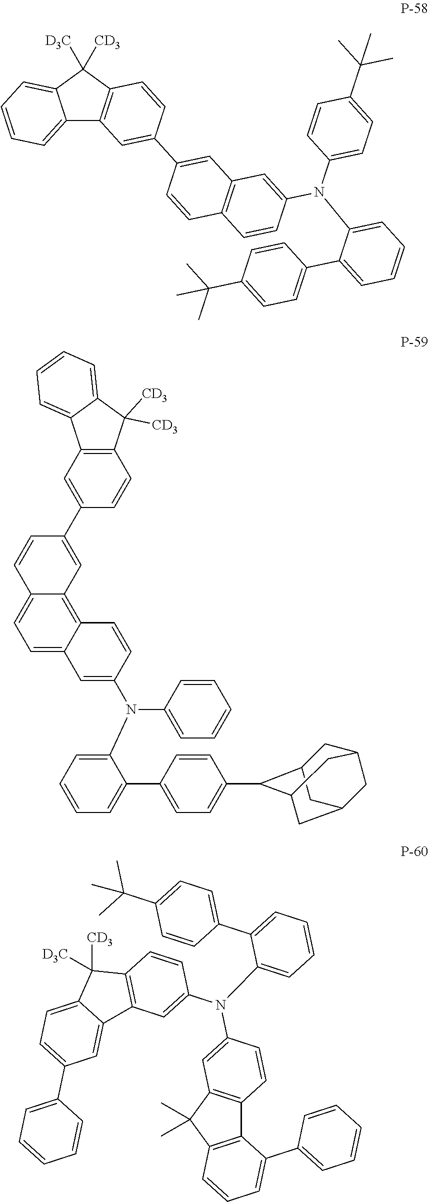 [SMILES: [2H]C([2H])([2H])C1(C([2H])([2H])[2H])c2ccccc2-c2cc(-c3ccc4ccc(N(c5ccc(C(C)(C)C)cc5)c5ccccc5-c5ccc(C(C)(C)C)cc5)cc4c3)ccc21 ; [2H]C([2H])([2H])C1(C([2H])([2H])[2H])c2ccccc2-c2cc(-c3ccc4ccc5cc(N(c6ccccc6)c6ccccc6-c6ccc(C7C8CC9CC(C8)CC7C9)cc6)ccc5c4c3)ccc21 ; [2H]C([2H])([2H])C1(C([2H])([2H])[2H])c2ccc(-c3ccccc3)cc2-c2cc(N(c3ccc4c(c3)C(C)(C)c3cccc(-c5ccccc5)c3-4)c3ccccc3-c3ccc(C(C)(C)C)cc3)ccc21]

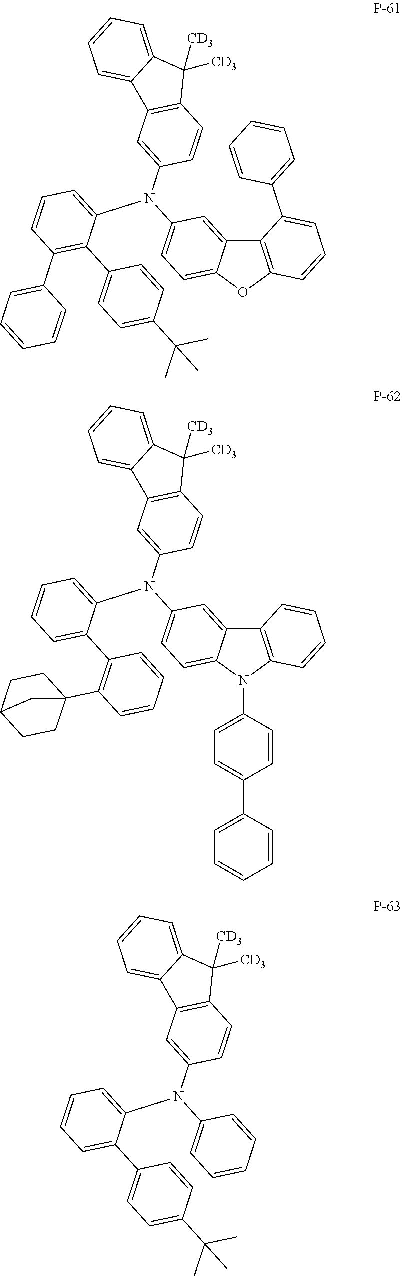

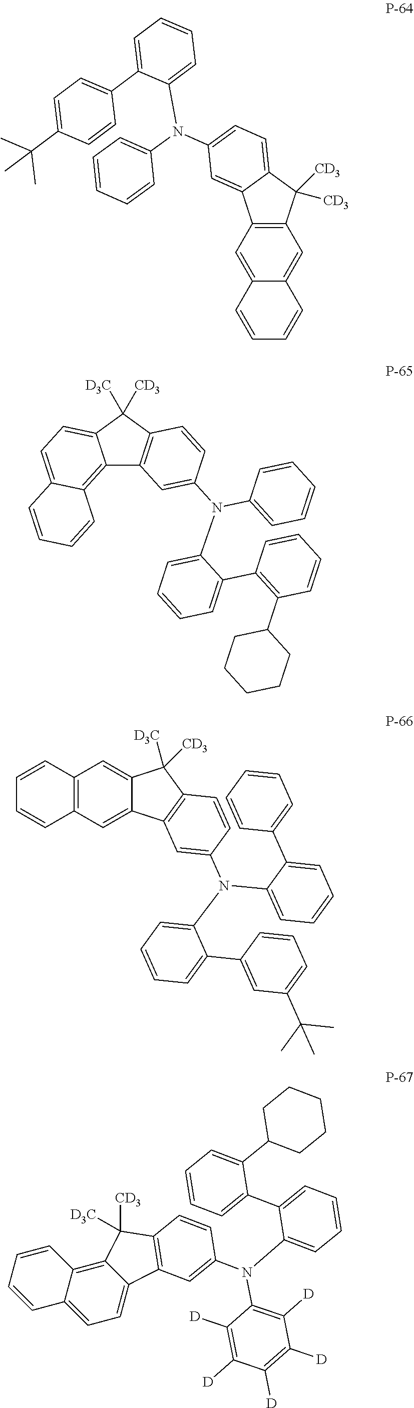

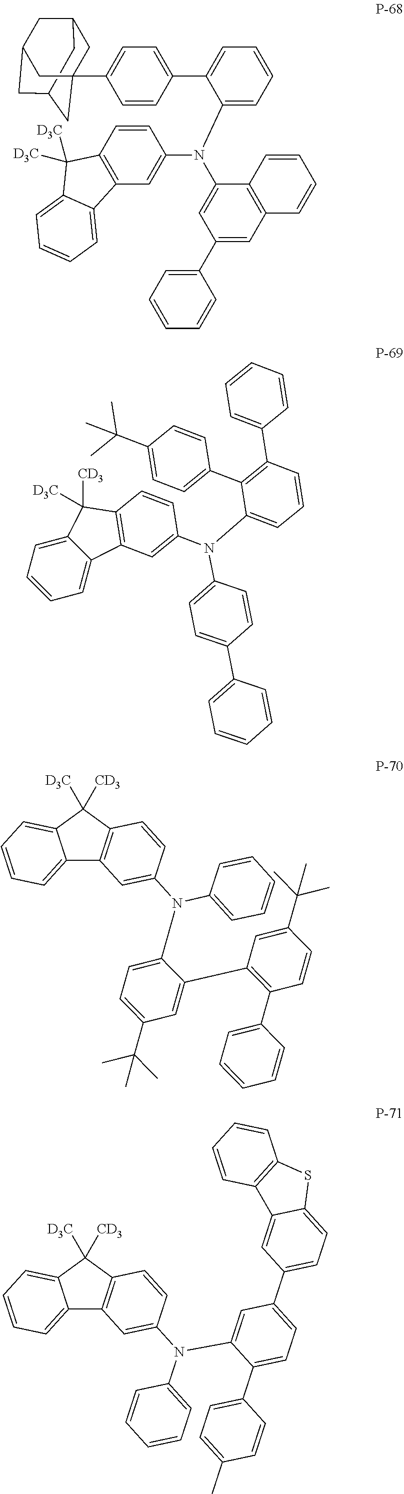

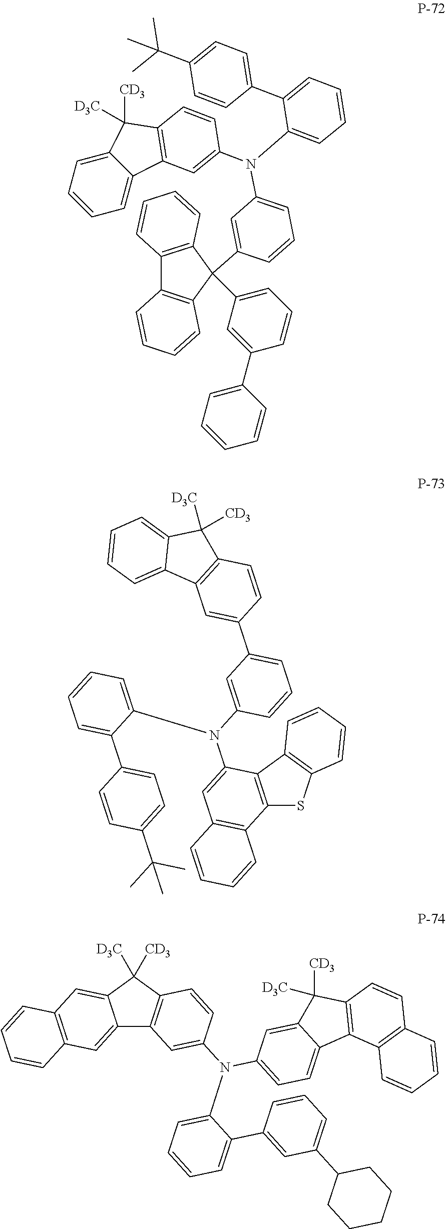 [SMILES: [2H]C([2H])([2H])C1(C([2H])([2H])[2H])c2ccccc2-c2cc(N(c3cccc(C4(c5cccc(-c6ccccc6)c5)c5ccccc5-c5ccccc54)c3)c3ccccc3-c3ccc(C(C)(C)C)cc3)ccc21 ; [2H]C([2H])([2H])C1(C([2H])([2H])[2H])c2ccccc2-c2cc(-c3cccc(N(c4ccccc4-c4ccc(C(C)(C)C)cc4)c4cc5ccccc5c5sc6ccccc6c45)c3)ccc21 ; [2H]C([2H])([2H])C1(C([2H])([2H])[2H])c2ccc(N(c3ccc4c(c3)C(C([2H])([2H])[2H])(C([2H])([2H])[2H])c3ccc5ccccc5c3-4)c3ccccc3-c3cccc(C4CCCCC4)c3)cc2-c2cc3ccccc3cc21]

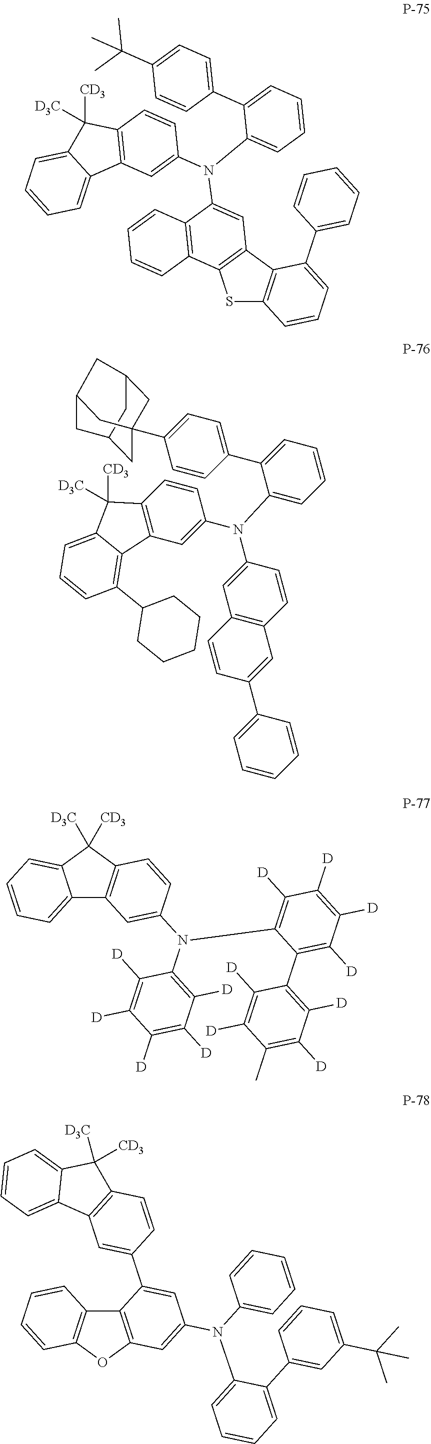

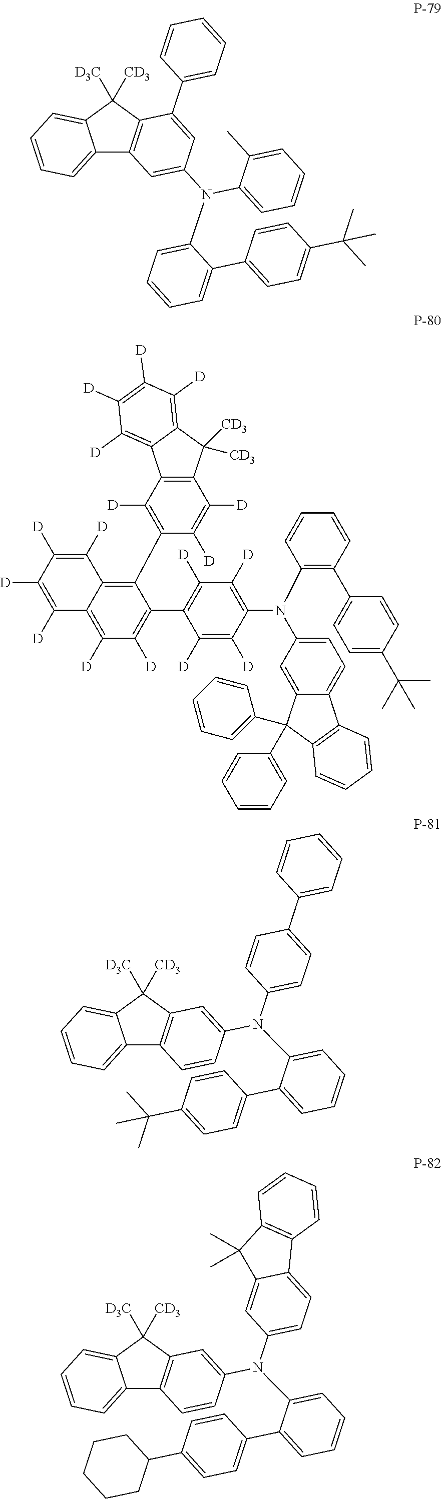

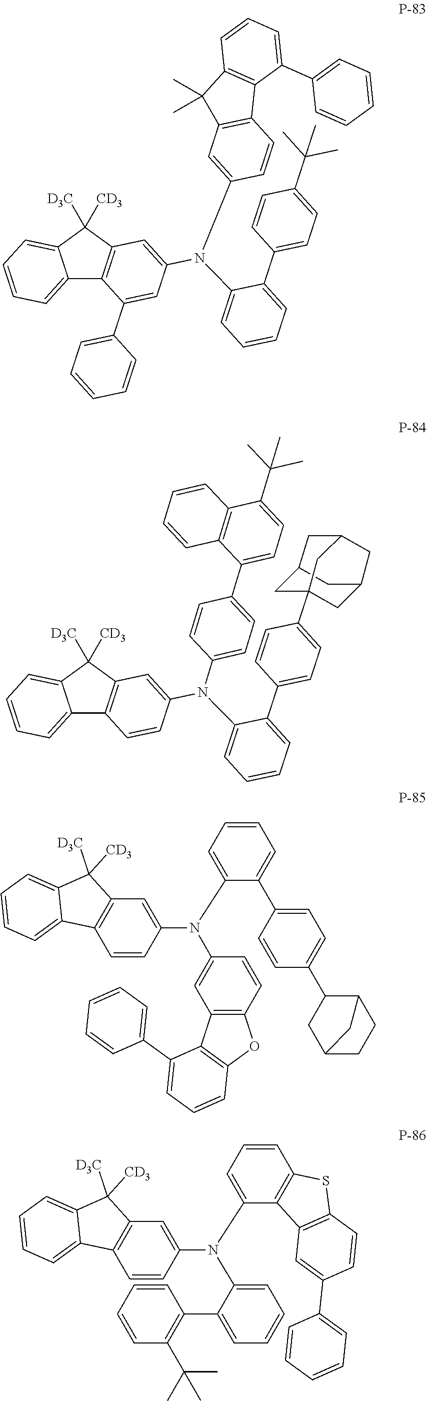

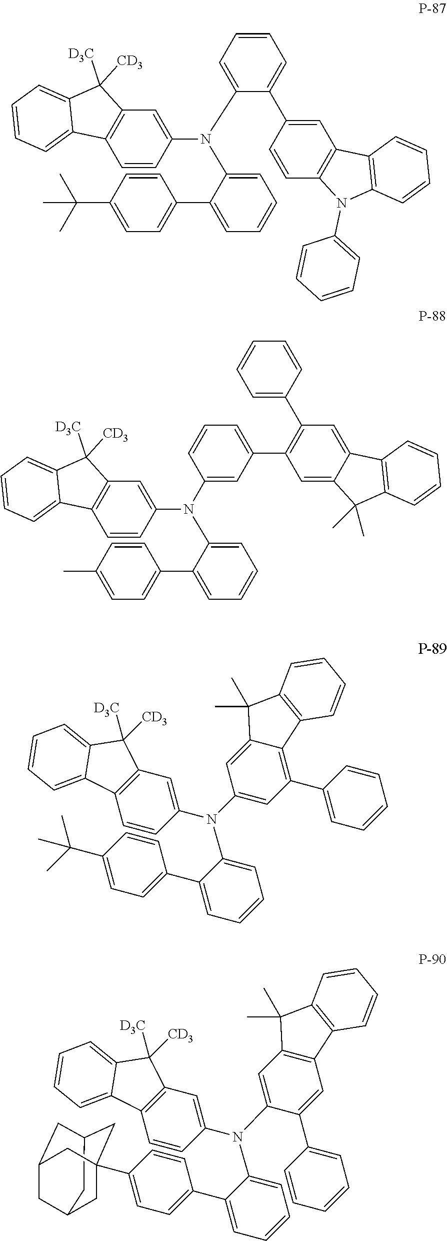

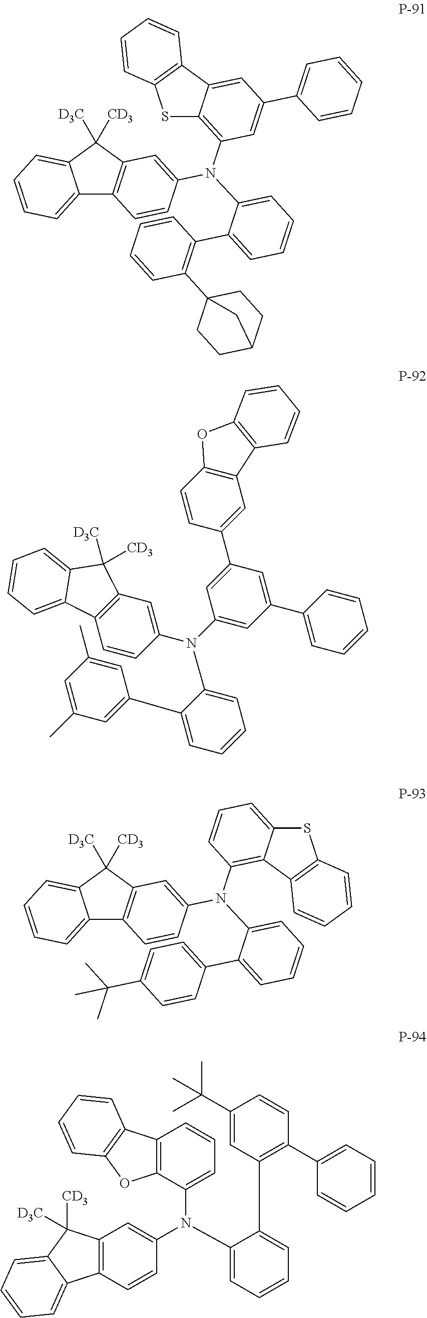

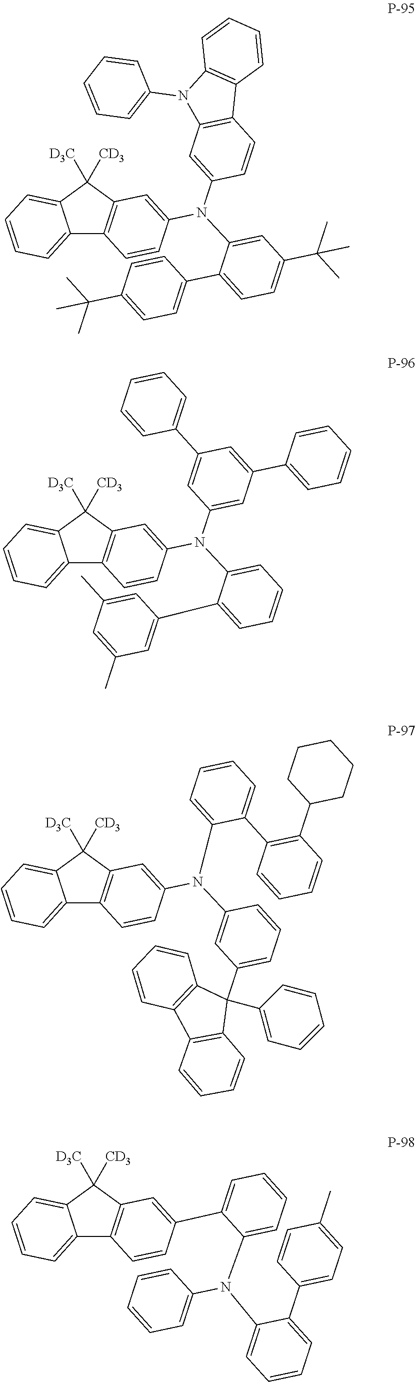 [SMILES: [2H]C([2H])([2H])C1(C([2H])([2H])[2H])c2ccccc2-c2ccc(N(c3ccc4c5ccccc5n(-c5ccccc5)c4c3)c3cc(C(C)(C)C)ccc3-c3ccc(C(C)(C)C)cc3)cc21 ; [2H]C([2H])([2H])C1(C([2H])([2H])[2H])c2ccccc2-c2ccc(N(c3cc(-c4ccccc4)cc(-c4ccccc4)c3)c3ccccc3-c3cc(C)cc(C)c3)cc21 ; [2H]C([2H])([2H])C1(C([2H])([2H])[2H])c2ccccc2-c2ccc(N(c3cccc(C4(c5ccccc5)c5ccccc5-c5ccccc54)c3)c3ccccc3-c3ccccc3C3CCCCC3)cc21 ; [2H]C([2H])([2H])C1(C([2H])([2H])[2H])c2ccccc2-c2ccc(-c3ccccc3N(c3ccccc3)c3ccccc3-c3ccc(C)cc3)cc21]

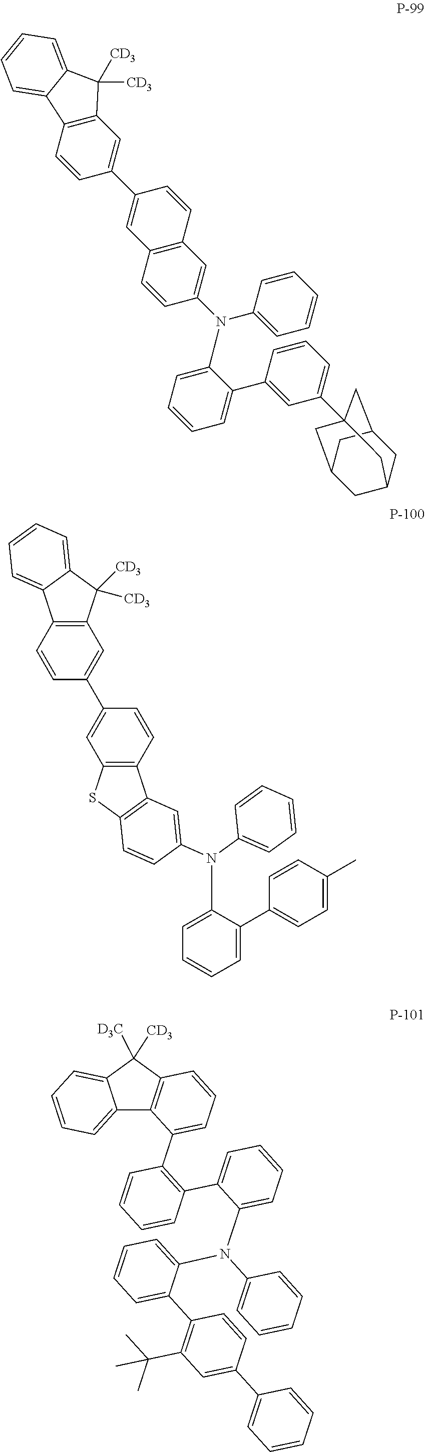 [SMILES: [2H]C([2H])([2H])C1(C([2H])([2H])[2H])c2ccccc2-c2ccc(-c3ccc4cc(N(c5ccccc5)c5ccccc5-c5cccc(C67CC8CC(CC(C8)C6)C7)c5)ccc4c3)cc21 ; [2H]C([2H])([2H])C1(C([2H])([2H])[2H])c2ccccc2-c2ccc(-c3ccc4c(c3)sc3ccc(N(c5ccccc5)c5ccccc5-c5ccc(C)cc5)cc34)cc21 ; [2H]C([2H])([2H])C1(C([2H])([2H])[2H])c2ccccc2-c2c(-c3ccccc3-c3ccccc3N(c3ccccc3)c3ccccc3-c3ccc(-c4ccccc4)cc3C(C)(C)C)cccc21]

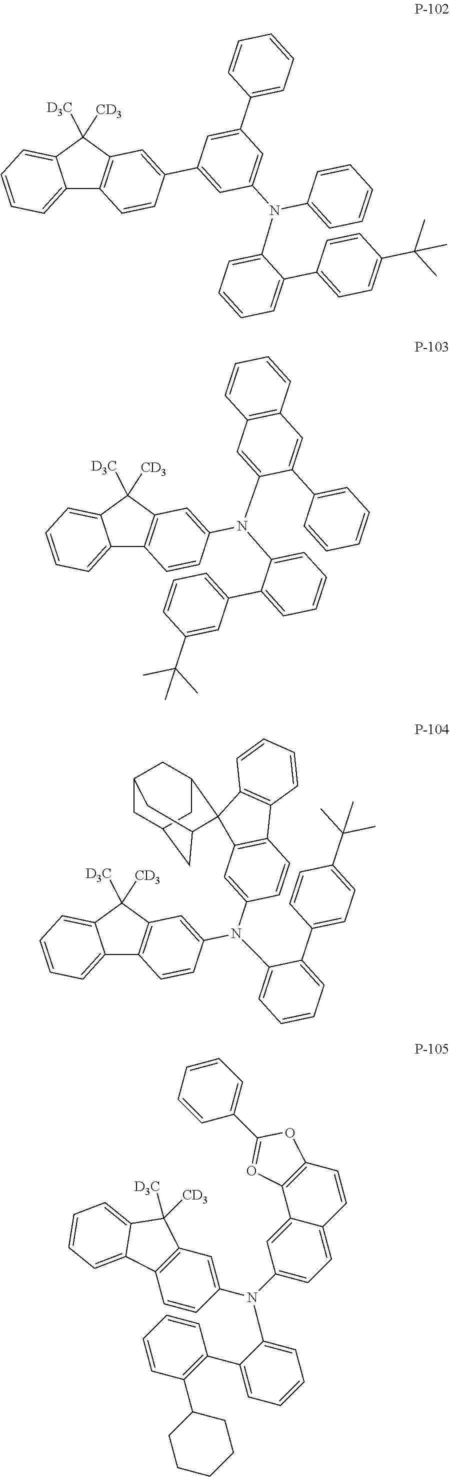

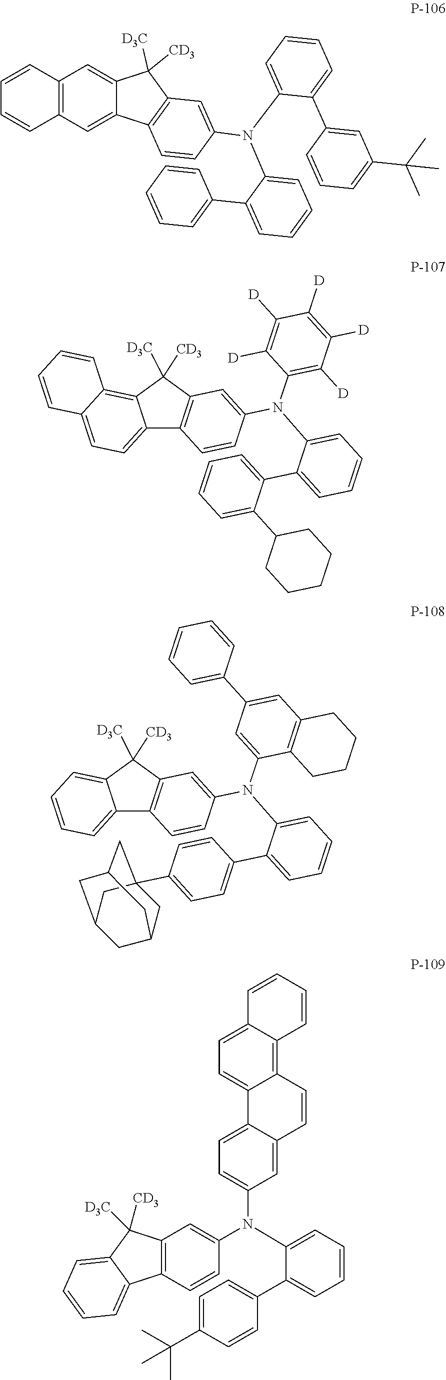 [SMILES: [2H]C([2H])([2H])C1(C([2H])([2H])[2H])c2cc(N(c3ccccc3-c3ccccc3)c3ccccc3-c3cccc(C(C)(C)C)c3)ccc2-c2cc3ccccc3cc21 ; [2H]c1c([2H])c([2H])c(N(c2ccc3c(c2)C(C([2H])([2H])[2H])(C([2H])([2H])[2H])c2c-3ccc3ccccc23)c2ccccc2-c2ccccc2C2CCCCC2)c([2H])c1[2H] ; [2H]C([2H])([2H])C1(C([2H])([2H])[2H])c2ccccc2-c2ccc(N(c3ccccc3-c3ccc(C45CC6CC(CC(C6)C4)C5)cc3)c3cc(-c4ccccc4)cc4c3CCCC4)cc21 ; [2H]C([2H])([2H])C1(C([2H])([2H])[2H])c2ccccc2-c2ccc(N(c3ccc4c(ccc5c6ccccc6ccc45)c3)c3ccccc3-c3ccc(C(C)(C)C)cc3)cc21]

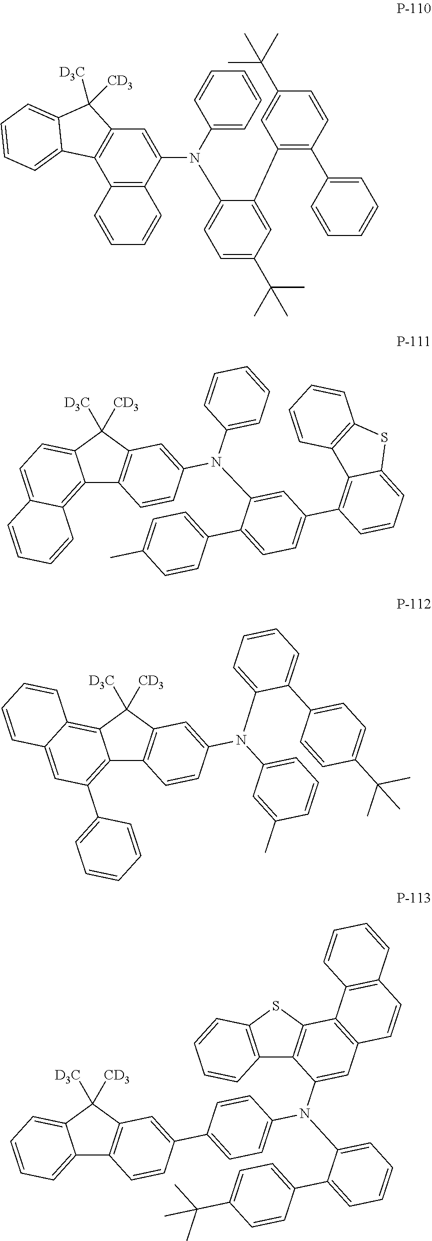 [SMILES: [2H]C([2H])([2H])C1(C([2H])([2H])[2H])c2ccccc2-c2c1cc(N(c1ccccc1)c1ccc(C(C)(C)C)cc1-c1cc(C(C)(C)C)ccc1-c1ccccc1)c1ccccc21 ; [2H]C([2H])([2H])C1(C([2H])([2H])[2H])c2cc(N(c3ccccc3)c3cc(-c4cccc5sc6ccccc6c45)ccc3-c3ccc(C)cc3)ccc2-c2c1ccc1ccccc21 ; [2H]C([2H])([2H])C1(C([2H])([2H])[2H])c2cc(N(c3cccc(C)c3)c3ccccc3-c3ccc(C(C)(C)C)cc3)ccc2-c2c(-c3ccccc3)cc3ccccc3c21 ; [2H]C([2H])([2H])C1(C([2H])([2H])[2H])c2ccccc2-c2ccc(-c3ccc(N(c4ccccc4-c4ccc(C(C)(C)C)cc4)c4cc5ccc6ccccc6c5c5sc6ccccc6c45)cc3)cc21]

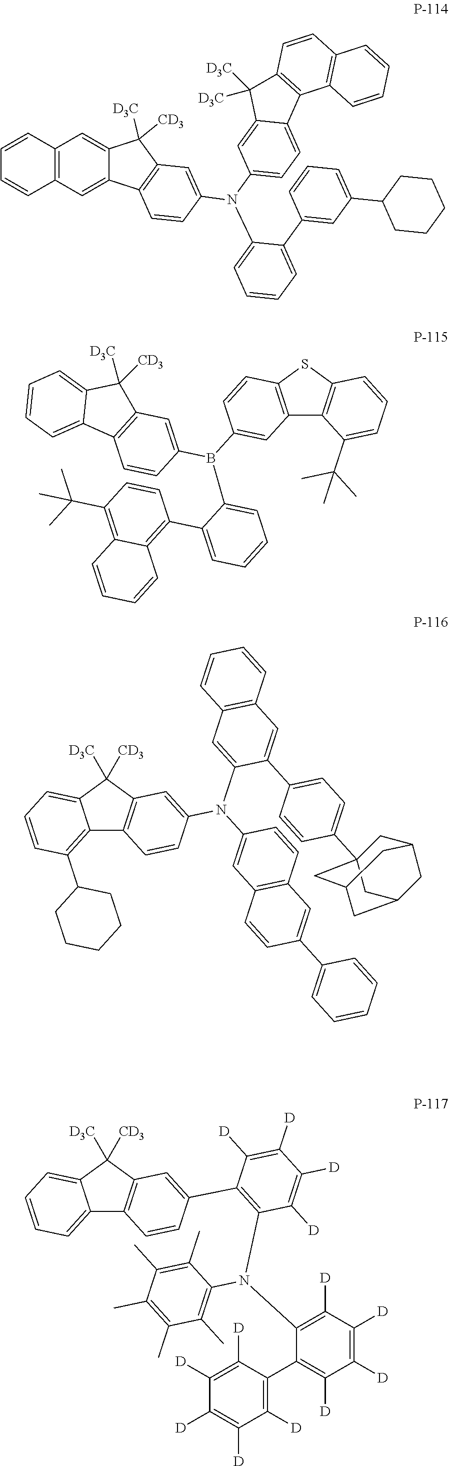

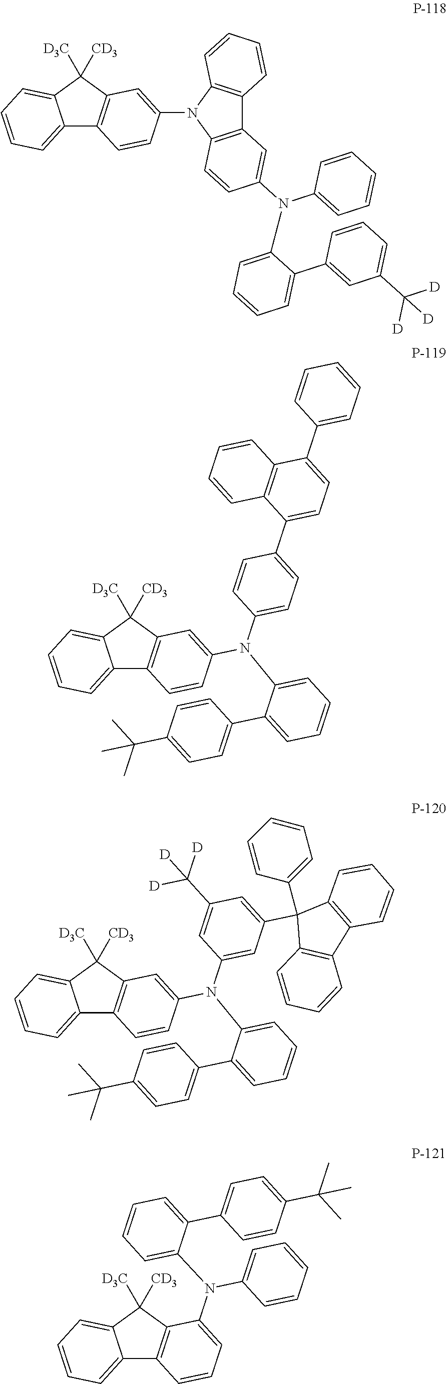

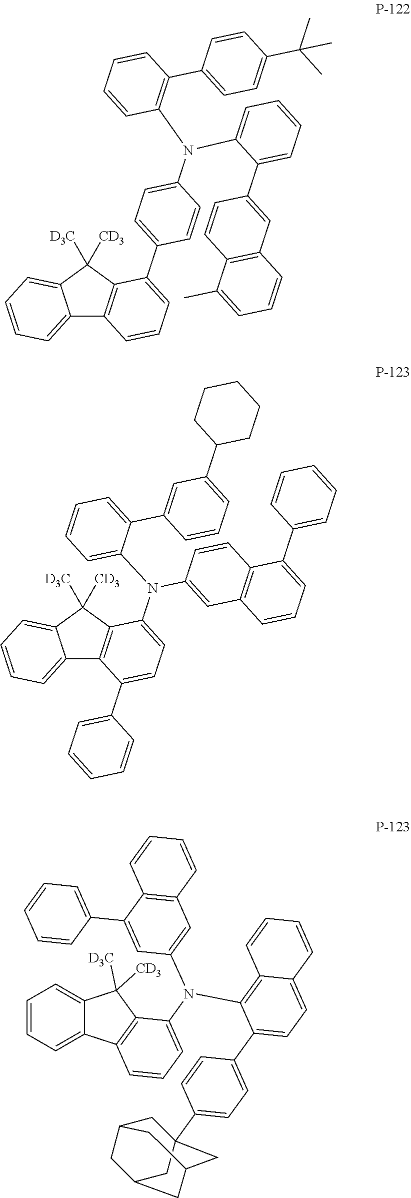 [SMILES: [2H]C([2H])([2H])C1(C([2H])([2H])[2H])c2ccccc2-c2cccc(-c3ccc(N(c4ccccc4-c4ccc(C(C)(C)C)cc4)c4ccccc4-c4ccc5c(C)cccc5c4)cc3)c21 ; [2H]C([2H])([2H])C1(C([2H])([2H])[2H])c2ccccc2-c2c(-c3ccccc3)ccc(N(c3ccc4c(-c5ccccc5)cccc4c3)c3ccccc3-c3cccc(C4CCCCC4)c3)c21 ; [2H]C([2H])([2H])C1(C([2H])([2H])[2H])c2ccccc2-c2cccc(N(c3cc(-c4ccccc4)c4ccccc4c3)c3c(-c4ccc(C56CC7CC(CC(C7)C5)C6)cc4)ccc4ccccc34)c21]

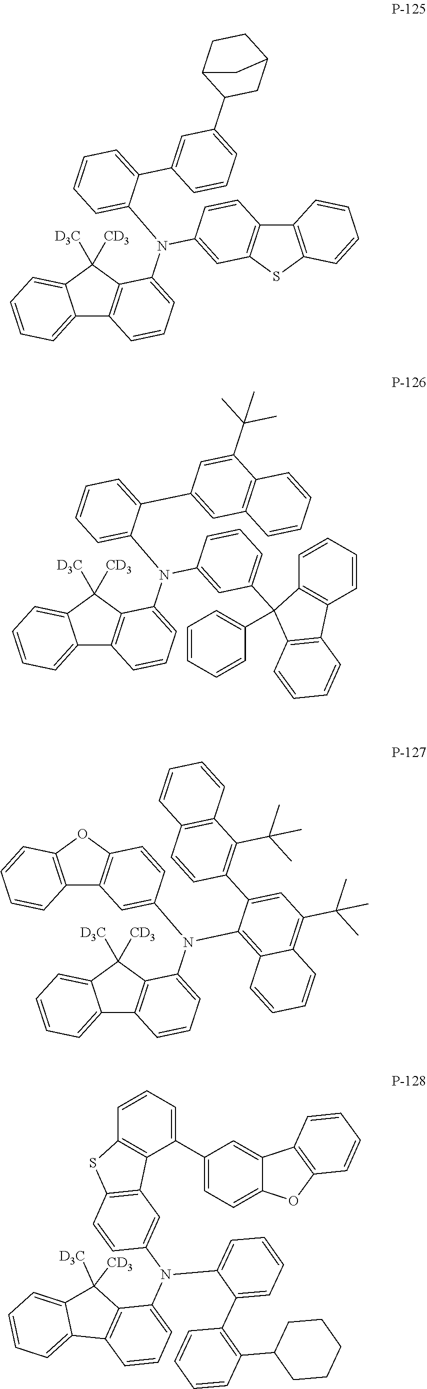

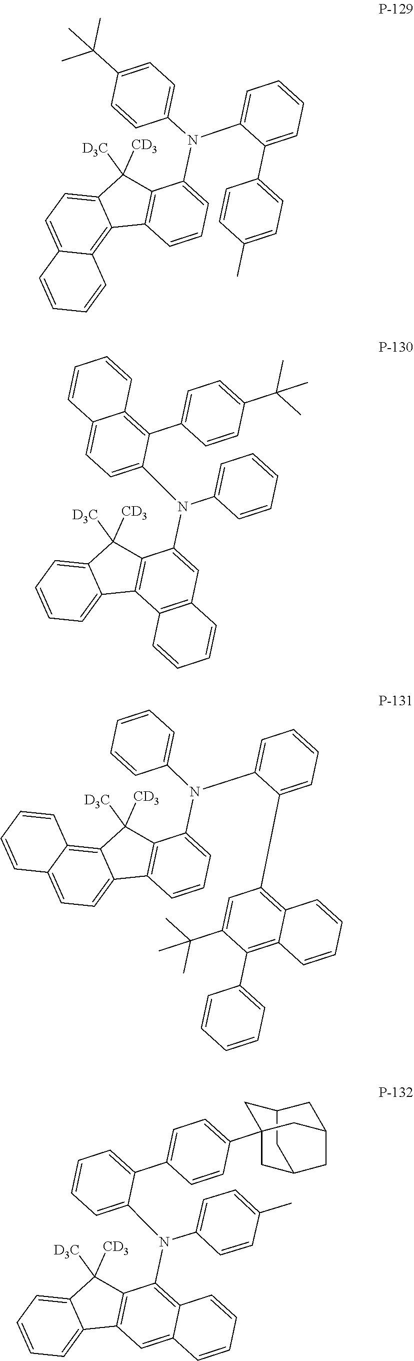 [SMILES: [2H]C([2H])([2H])C1(C([2H])([2H])[2H])c2ccc3ccccc3c2-c2cccc(N(c3ccc(C(C)(C)C)cc3)c3ccccc3-c3ccc(C)cc3)c21 ; [2H]C([2H])([2H])C1(C([2H])([2H])[2H])c2ccccc2-c2c1c(N(c1ccccc1)c1ccc3ccccc3c1-c1ccc(C(C)(C)C)cc1)cc1ccccc21 ; [2H]C([2H])([2H])C1(C([2H])([2H])[2H])c2c(cccc2N(c2ccccc2)c2ccccc2-c2cc(C(C)(C)C)c(-c3ccccc3)c3ccccc23)-c2ccc3ccccc3c21 ; [2H]C([2H])([2H])C1(C([2H])([2H])[2H])c2ccccc2-c2cc3ccccc3c(N(c3ccc(C)cc3)c3ccccc3-c3ccc(C45CC6CC(CC(C6)C4)C5)cc3)c21]

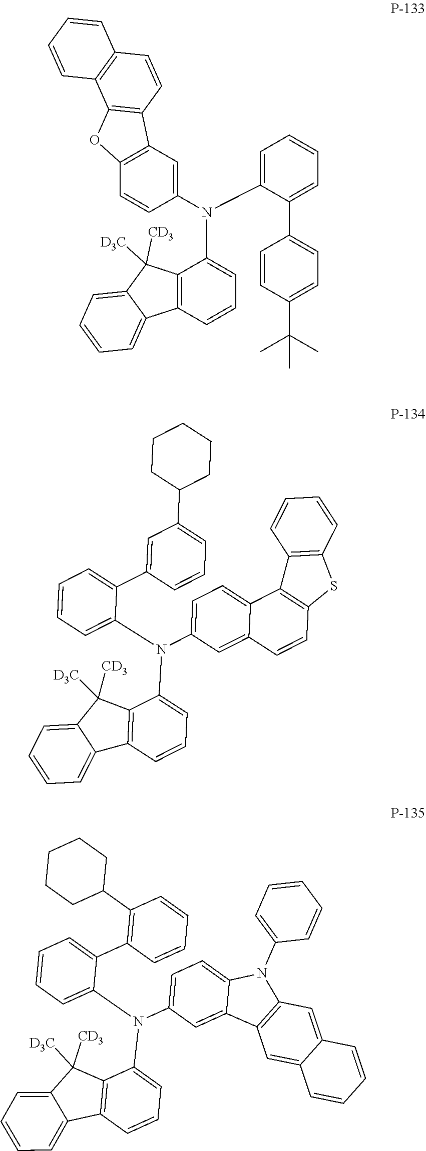 [SMILES: [2H]C([2H])([2H])C1(C([2H])([2H])[2H])c2ccccc2-c2cccc(N(c3ccc4oc5c6ccccc6ccc5c4c3)c3ccccc3-c3ccc(C(C)(C)C)cc3)c21 ; [2H]C([2H])([2H])C1(C([2H])([2H])[2H])c2ccccc2-c2cccc(N(c3ccc4c(ccc5sc6ccccc6c54)c3)c3ccccc3-c3cccc(C4CCCCC4)c3)c21 ; [2H]C([2H])([2H])C1(C([2H])([2H])[2H])c2ccccc2-c2cccc(N(c3ccc4c(c3)c3cc5ccccc5cc3n4-c3ccccc3)c3ccccc3-c3ccccc3C3CCCCC3)c21]

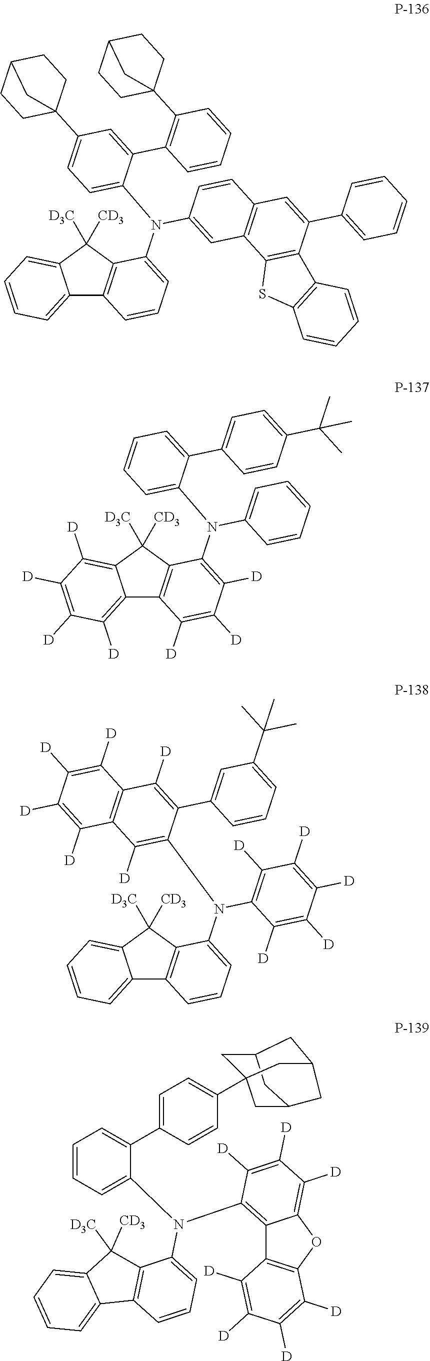

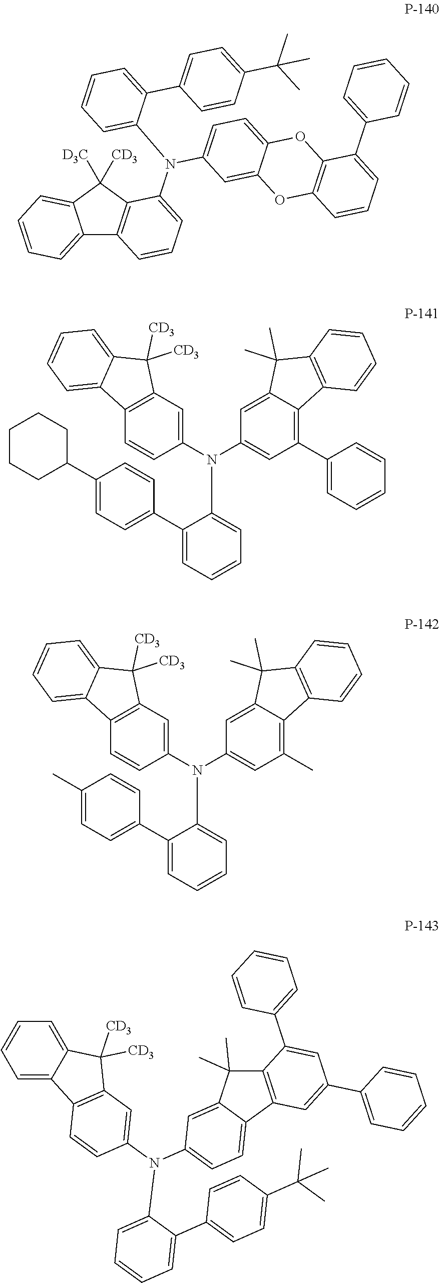

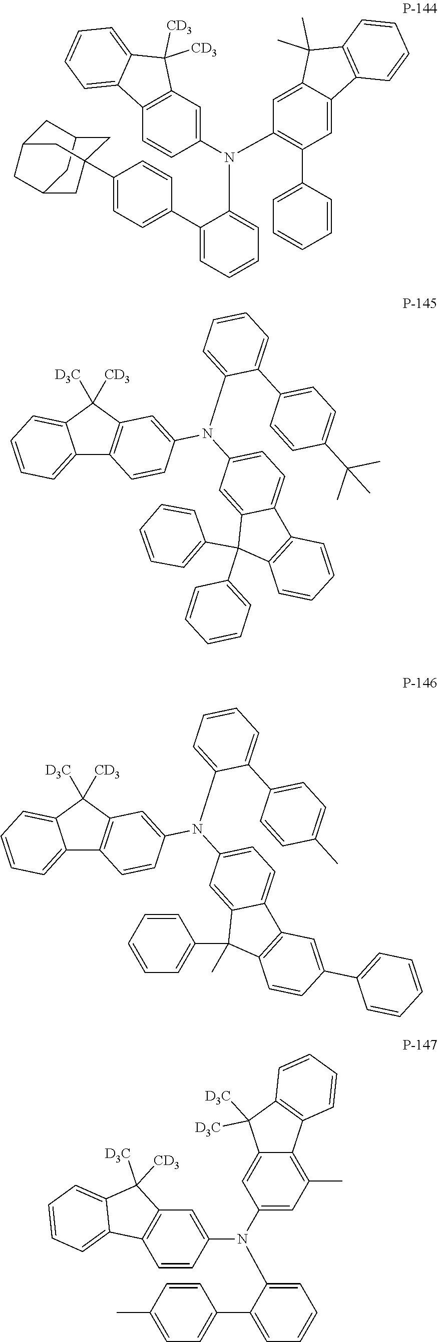

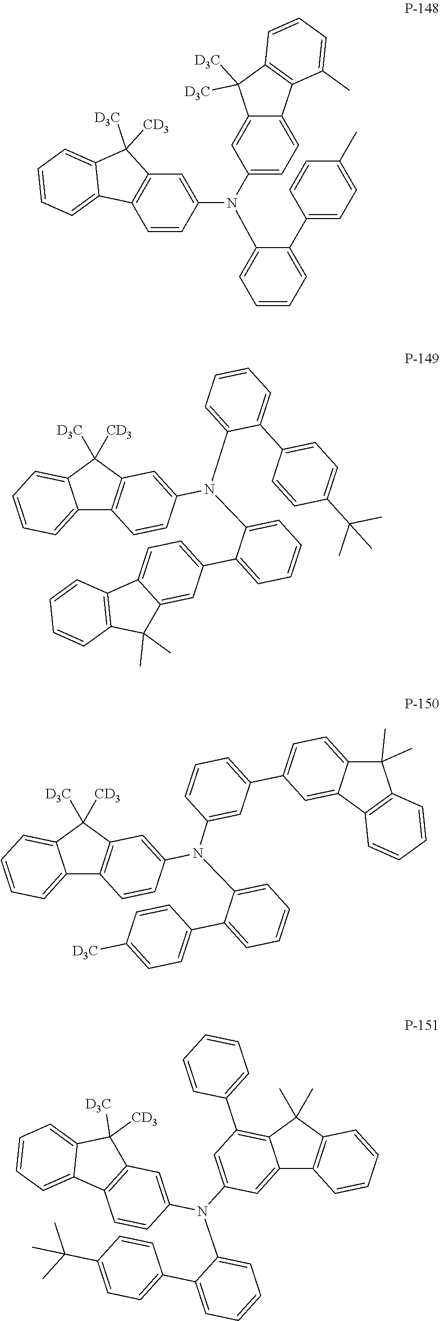

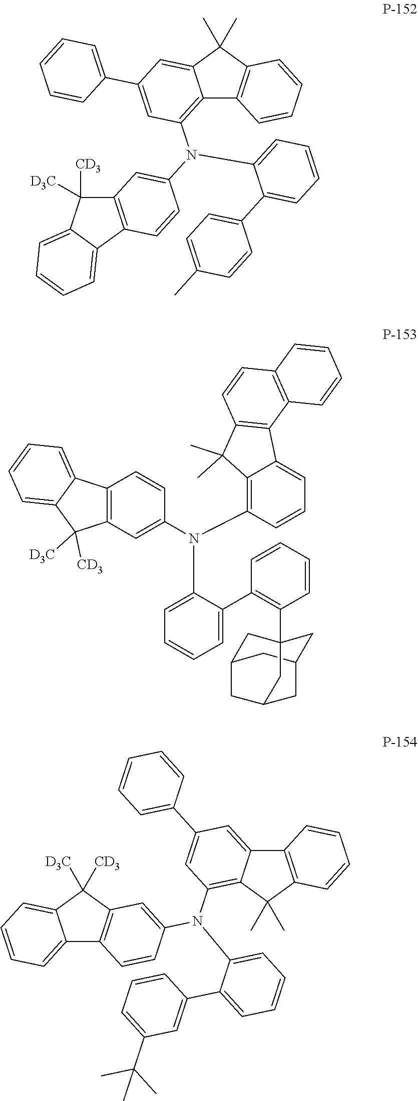 [SMILES: [2H]C([2H])([2H])C1(C([2H])([2H])[2H])c2ccccc2-c2ccc(N(c3ccccc3-c3ccc(C)cc3)c3cc(-c4ccccc4)cc4c3-c3ccccc3C4(C)C)cc21 ; [2H]C([2H])([2H])C1(C([2H])([2H])[2H])c2ccccc2-c2ccc(N(c3ccccc3-c3ccccc3C34CC5CC(CC(C5)C3)C4)c3cccc4c3C(C)(C)c3ccc5ccccc5c3-4)cc21 ; [2H]C([2H])([2H])C1(C([2H])([2H])[2H])c2ccccc2-c2ccc(N(c3ccccc3-c3cccc(C(C)(C)C)c3)c3cc(-c4ccccc4)cc4c3C(C)(C)c3ccccc3-4)cc21]

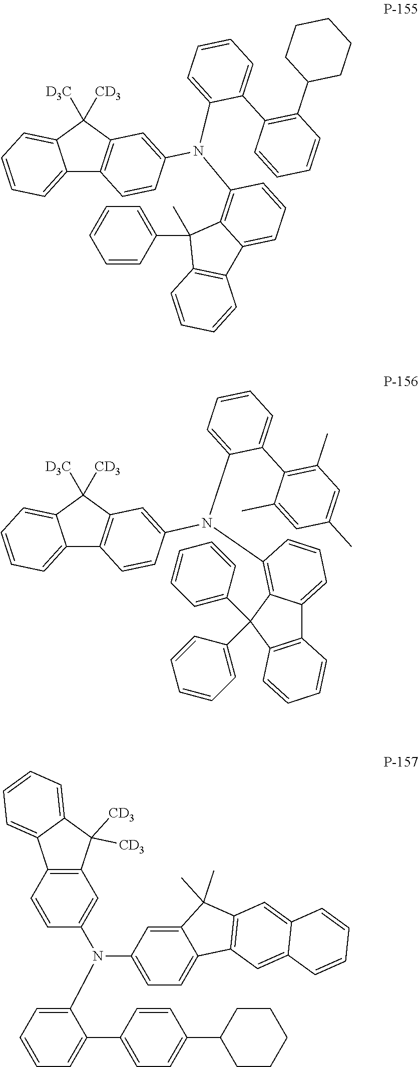 [SMILES: [2H]C([2H])([2H])C1(C([2H])([2H])[2H])c2ccccc2-c2ccc(N(c3ccccc3-c3ccccc3C3CCCCC3)c3cccc4c3C(C)(c3ccccc3)c3ccccc3-4)cc21 ; [2H]C([2H])([2H])C1(C([2H])([2H])[2H])c2ccccc2-c2ccc(N(c3ccccc3-c3c(C)cc(C)cc3C)c3cccc4c3C(c3ccccc3)(c3ccccc3)c3ccccc3-4)cc21 ; [2H]C([2H])([2H])C1(C([2H])([2H])[2H])c2ccccc2-c2ccc(N(c3ccc4c(c3)C(C)(C)c3cc5ccccc5cc3-4)c3ccccc3-c3ccc(C4CCCCC4)cc3)cc21]

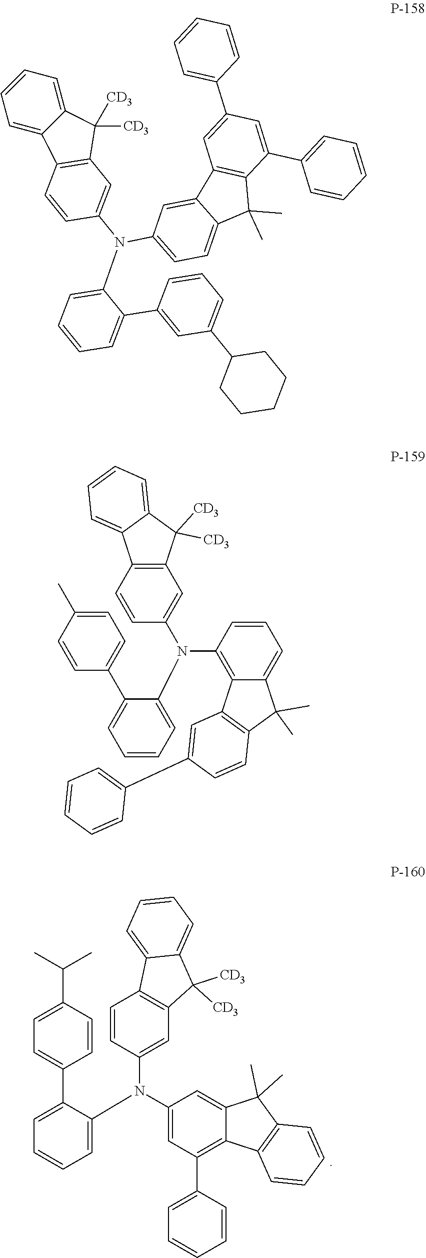 [SMILES: [2H]C([2H])([2H])C1(C([2H])([2H])[2H])c2ccccc2-c2ccc(N(c3ccc4c(c3)-c3cc(-c5ccccc5)cc(-c5ccccc5)c3C4(C)C)c3ccccc3-c3cccc(C4CCCCC4)c3)cc21 ; [2H]C([2H])([2H])C1(C([2H])([2H])[2H])c2ccccc2-c2ccc(N(c3ccccc3-c3ccc(C)cc3)c3cccc4c3-c3cc(-c5ccccc5)ccc3C4(C)C)cc21 ; [2H]C([2H])([2H])C1(C([2H])([2H])[2H])c2ccccc2-c2ccc(N(c3cc(-c4ccccc4)c4c(c3)C(C)(C)c3ccccc3-4)c3ccccc3-c3ccc(C(C)C)cc3)cc21]

8. An organic electronic element comprising an anode, a cathode and an organic material layer formed between the anode and the cathode, wherein the organic material layer comprises a single compound or 2 or more compounds represented by Formula 1 of claim 1.

9. The organic electronic element according to claim 8, wherein the organic material layer comprises at least one of a hole injection layer, a hole transport layer, an emitting auxiliary layer, an emitting layer, an electron transport auxiliary layer, an electron transport layer, and an electron injection layer.

10. The organic electronic element according to claim 8, wherein the organic material layer is an emitting auxiliary layer.

11. The organic electronic element according to claim 8, further comprising a light efficiency enhancing layer formed on at least one surface of the anode and the cathode, the surface being opposite to the organic material layer.

12. The organic electronic element according to claim 8, wherein the organic material layer comprises 2 or more stacks comprising a hole transport layer, an emitting layer, and an electron transport layer sequentially formed on an anode.

13. The organic electronic element according to claim 12, wherein the organic material layer further comprises a charge generation layer formed between the 2 or more stacks.

14. An electronic device comprising a display device comprising the organic electronic element of claim 8; and a control unit for driving the display device.

15. The electronic device according to claim 14, wherein the organic electronic element is at least one of an OLED, an organic solar cell, an organic photo conductor (OPC), organic transistor (organic TFT) and an element for monochromic or white illumination.

16. A method of reusing the compound of Formula 1 of claim 1 comprising:
recovering a crude organic light emitting material comprising the compound of Formula 1 from a deposition apparatus used in a process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.
